(19) United States
(12) Patent Application Publication
(10) Pub. No.: US 2019/0092876 A1
(43) Pub. Date: Mar. 28, 2019

(54) T-CELL RECEPTOR MIMIC (TCRM) ANTIBODIES

(71) Applicant: Cancer Research Technology Limited, London (GB)

(72) Inventors: Alison Banham, London Greater London (GB); Demin Li, London Greater London (GB)

(21) Appl. No.: 15/756,059

(22) PCT Filed: Aug. 26, 2016
(86) PCT No.: PCT/GB2016/052674

§ 371 Date: Feb. 27, 2018

(30) Foreign Application Priority Data
Aug. 28, 2015 (GB) 1515351.3

(51) Int. Cl. C07K 16/32; A61K 39/395; A61K 45/06; A61K 51/10; G01N 33/574; A61P 35/00

(52) U.S. Cl. CPC C07K 16/32; A61K 39/39558; A61K 45/06; A61K 51/1045; G01N 33/57492; A61P 35/00; A61K 2039/505; C07K 2317/92; C07K 2317/77; C07K 2317/73; C07K 2317/732; C07K 2317/734; C07K 2317/34

(57) ABSTRACT

The present invention provides an antibody which binds to human p53 tumour suppressor protein residues 65-73 (human p53₆₅₋₇₃), as shown in SEQ ID NO: 1, when presented by the MHC class I protein Human Leukocyte Antigen-A*0201 (HLA-A*0201).

### FIELD OF THE INVENTION

The present invention relates to T-cell receptor mimic (TCRm) antibodies, and their use in the therapy of cancer.

### BACKGROUND TO THE INVENTION

Cancer comprises many distinct disease entities and represents one of the leading causes of death in developed countries. Therapeutic monoclonal antibodies are among the most effective drugs used in modern cancer medicine and their use is expanding rapidly.[1] Examples include the CD20-targeting antibody Rituximab[2], that is commonly used to treat B-cell malignancies and Herceptin[3], which targets the HER-2 protein in breast cancer. Importantly, they represent a proven biological therapeutic technology with an established methodology for manufacture, delivery, and regulatory approval.

Classical therapeutic antibodies commonly target cell surface or secreted antigens. However, intracellular proteins are degraded by proteasome-dependent and independent mechanisms, resulting in the generation of peptides for surface presentation by major histocompatibility complex (MHC) class I.[4] This presentation of peptides derived from intracellular proteins on the cell surface is part of the normal cellular process enabling the recognition of intracellular antigens by the immune system. In particular recognition by CD8⁺ T cells whose T-cell receptors (TCRs) bind the MHC class I-presented peptides to enable T-cell targeted killing of cells expressing foreign antigens. Antibodies mimicking this ability of T cells to recognise MHC class I-presented peptides have been generated against a range of different intracellular antigens, reviewed by [5-7]. The utility of such antibodies, whose epitope comprises both the target peptide and MHC molecule, will be restricted to a specific HLA (human leukocyte antigen) haplotype. Thus common haplotypes such as HLA-A*0201 (HLA-A2), which is found in approximately 50% of Caucasians, and HLA-A24, which is frequent in Asian populations, are favoured for broad applicability.

Phage antibodies with TCR-like specificity, recognising MHC class I/peptide complexes, tended to be low affinity and lacked immune effector functions. These were initially used to study antigen presentation and TCR-peptide-MHC interactions.[8] Using MHC tetramers as immunogens has reproducibly enabled the production of high affinity ‘TCR mimic’ (TCRm) antibodies against multiple targets using hybridoma technology.[5] Several antibodies against cancer targets have in vivo activity against tumours by mediating immune effector mechanisms such as complement-dependent cytotoxicity (CDC) and/or antibody-dependent cellular cytotoxicity (ADCC), reviewed by [6]. In addition some TCRm antibodies have been reported to kill via immune effector independent mechanisms that induce apoptosis of tumour cells.[9]

One of the most extensively studied tumour-associated antigens is the tumour suppressor p53, whose widespread deregulation and involvement in malignant transformation make it an almost universal target for the immunotherapy of cancer. In 2009 it was estimated that there were approximately 11 million cancer patients whose tumour had a TP53 inactivating mutation, and a further 11 million patients where the p53 pathway was abrogated via another mechanism.[10] Germ line TP53 mutations cause Li-Fraumeni Syndrome (LFS), a rare type of inherited cancer pre-disposition.[11] Interestingly a mother and daughter with strong wild type p53 expression in most normal epithelial and mesenchymal cells were also pre-disposed to develop cancer at an early age.[12] TP53 gene mutations, which generally increase expression of the mutated protein, are detected in over 50% of human malignancies.[13]

Studies of wild type p53 have identified its normal functional roles in a wide range of biological processes including DNA repair, metabolism, senescence, apoptosis and cell cycle arrest.[14] Mutations in TP53 act to abrogate its normal function and importantly can also lead to gain of novel transforming function.[15] As p53 forms tetramers, the more abundant mutant protein can functionally interact with and inactivate the remaining wild type protein. Notably, mice carrying TP53 mutations exhibit more aggressive tumours than mice that are p53 null.[16] Approximately 80% of TP53 mutations represent missense mutations that stabilise the protein, leading to accumulation of the full-length p53 protein in tumour cells. These, p53 mutations are highly diverse and commonly only alter a single amino acid. Furthermore, 8% of TP53 mutations (most frequently R196X and R213X) cause p53 C-terminal protein truncation, as a consequence of premature termination of translation.[14]

Regulation of p53 expression and epitope presentation is complex. Protein levels are commonly regulated by interactions with the MDM2 and MDMX proteins, which regulate p53 targeting for ubiquitination and degradation. However, additional regulatory mechanisms include post-translational modifications (e.g. phosphorylation and acetylation) and interaction with other cofactors e.g. ASPPs (apoptosis-stimulating protein of p53). Thus p53 in tumours can be regulated by other mechanisms, including MDM2 overexpression, human papilloma virus infection and p14^{ARF} mutations. These mechanisms of p53 regulation and the existing therapeutic strategies to drug the p53 pathway, particularly those that activate or restore the pathway, have recently been extensively reviewed.[10, 14, 17, 18] High-copy numbers of wild type p53 peptide-MHC class I complexes on tumour cells, as compared to low copies on normal cells, have already been reported by several studies.[19-21] This can also result from the increased turnover and thus processing of p53, which occurs in tumours with more modest steady-state levels of expression.[20]

Immunotherapy strategies to target MHC class I-presented tumour peptides, such as those derived from p53, include vaccines, recombinant TCRs and TCRm antibodies. Missense mutations in TP53 can lead to an accumulation of p53 protein in the cytosol, which for some, as yet undefined, reason leads to enhanced processing (of both wild type and mutant peptides) by the antigen processing machinery.[22] Evidence from studying the humoural immune responses in cancer patients is that they recognise both wild type and mutant p53 epitopes, without mutant p53 containing immunodominant epitopes that are absent in the wild type protein.[23] Mutant p53 represents a tumour specific target and CD8⁺ cytotoxic T lymphocytes (CTLs) can be raised against mutant p53 derived epitopes.[24] The use of a mutated TP53 allele in a DNA vaccine was able to protect against tumour growth, however, no protection was conferred against tumours with other TP53 mutations or overexpressed wild type p53.[25] The diversity of TP53 mutations, scarcity of mutant p53-derived T-cell epitopes[24, 26] and alternative mechanisms that regulate the wild type p53 protein make immunotherapeutic strategies targeting wild type p53 epitopes more broadly applicable in a clinical setting, reviewed by [27-29].

This raises important safety considerations, as p53 is a ‘self’ protein that is expressed in normal tissues. While loss of p53 can have some impact on embryonic development[30], p53 functionality is not absolutely required for normal adult cell growth and differentiation. Furthermore, p53 is usually expressed at very low levels in normal cells[31, 32], although some immunolabelling of normal tissues, including normal lymphocytes, has been reported.[33-35] Importantly, CTLs recognising wild type p53 can discriminate between p53⁺ tumour cells and normal tissues.[36] Both CTLs and T helper (Th) cells directed against wild type p53 also eradicated tumours in vivo in mice with wild type p53 expression, without damage to normal tissues.[37, 38] It is this differential presentation of p53 epitopes by normal and malignant cells and its on-going requirement for tumour growth and survival, which form the rational basis of p53 targeted immunotherapy strategies. [28]

Importantly high tumour levels of p53 are not a prerequisite for tumour killing by CTLs against wild type p53 peptides, and such CTLs were able to kill tumours with low level p53 protein expression. A high p53 turnover rate, leading to enhanced tumoural presentation of peptides by MHC class I, has been proposed as an important factor governing wild type p53 epitope presentation.[20] Interestingly T-cell recognition of tumours without detectable p53 protein expression has been reported within the context of human papilloma virus infection, where enhanced p53 proteasomal degradation occurs.[39] Thus, both wild type and mutated p53 are among the top tumour antigens prioritised by a national cancer institute pilot project to accelerate translational research.[40]

Vaccination studies utilising a variety of wild type p53 peptides (HLA-A2 restricted peptides comprising p53 amino acids 65-73, 149-157, 187-197, 217-255 and 264-272) and different vaccine delivery systems have been taken through to clinical trials.[28, 29] While the vaccines were safe, able to induce anti-p53 immune responses, and with some patients achieving stable disease, the present inventors are unaware of clinical responses with a significant reduction in tumour burden.[28, 29] There are many potential reasons for the lack of clinical effectiveness of cancer vaccines, including immune suppression in cancer patients. Thus future efforts propose to include immunopotentiating clinical combination strategies.

While the function of TCRs is to bind MHC class-1 presented peptides, there have been obstacles facing the engineering of recombinant TCRs for use in cancer therapy.[41, 42] These have included primarily difficulties in expressing recombinant TCRs, their naturally low affinity and the inherent degeneracy of T-cell recognition for multiple peptide-MHC complexes from different molecules.[43] Recent developments enabling the production of high affinity TCRs that retain their specificity, including soluble monoclonal TCRs, have generated new reagents for cancer therapy. [44-46] These include immune-mobilising monoclonal TCRs against cancer (ImmTACs) in which a bispecific molecule, comprising a tumour targeting soluble monoclonal TCR fused to a CD3 antibody fragment, effectively overcomes immune tolerance by redirecting T cells to kill the cancer cells.[46] There is also significant interest in using both recombinant TCRs and TCRm/TCR-like antibodies in cellular therapies, to provide the tumour antigen targeting moiety on chimeric antigen receptor (CAR) modified T cells.[47]

High-affinity wild type p53 TCRs have been proposed as future therapeutic agents.[27] A recombinant single-chain moderately high affinity TCR against p53₁₄₉₋₁₅₇, derived from HLA-A2 transgenic mice, was capable of selectively delivering a cytotoxic molecule to antigen presenting cells expressing the target p53 peptide.[48] A TCR recognising p53₂₆₄₋₂₇₂ presented by HLA-A2 was able to reduce lung metastasis in an experimental model, when fused to IL-2, and has been used to stain human tumours.[49, 50] This p53 TCR-IL-2 fusion, designated ALT-801, has progressed to a phase I trial where it had a short half-life but could be administered safely and with immunological changes of potential anti-tumour relevance.[51] A phase 1b/2 escalation clinical trial in metastatic melanoma has recently been completed and further phase 2 trials in bladder cancer are ongoing. When linked to an IgG1 H constant domain, the same p53₂₆₄₋₂₇₂ TCR exhibited potent anti-tumour activity in vivo, enabling cell killing by antibody-dependent cytotoxicity.[52] However, another group studied a soluble TCR high-affinity p53₂₆₄₋₂₇₂-reactive TCR, derived from HLA-A2 transgenic mice, that showed no significant correlation between p53 expression in tumours and recognition by anti-p53 TCR-transduced T cells, raising safety concerns about targeting this epitope.[39] The production of a TCRm antibody against the HLA-A2 presented p53₂₆₄₋₂₇₂ epitope has been described in WO 2005/116072. The disclosure provides a method of generating and isolating TCRm antibodies using, in one approach, recombinantly-expressed heavy and light chains of the HLA-A2 Class I molecule, incubated with the synthetic peptide of p53₂₆₄₋₂₇₂ (LLGRNSFEV). The resultant complexes were subsequently multimerised by biotin-streptavidin linkages, to form a stable tetramer used for immunisation. However, the anti-tumour activity of the resultant TCRm antibody was not described, and, to the best of the inventors' knowledge, this reagent has not been further characterised in the scientific literature. The same method and resultant antibody is also described in WO 2007/030451 (in addition to the generation of TCRm antibodies against other, non-p53-derived, HLA-A2-presented epitopes).

Numerous other TCRm antibodies are described in the art, for example in WO 2003/068201, WO 2003/070752, EP 2329814, US 2009/0304679, WO 2012/129520, WO 2012/135854, WO 2012/109659, WO 2010/065962, WO 2014 011489 and WO 2014/011489. However, these antibodies are specific for HLA-A2-presented non-p53-derived epitopes (for example, those derived from gp100, tyrosinase, Her2 and p68 helicase, among many other targets)

It is to be noted that the p53₂₆₄₋₂₇₂ epitope (targeted by the TCRm antibody of WO 2005/116072 and WO 2007/030451) is located C-terminal to the most common mutations leading to premature termination of translation of p53 (R196X and R213X). Accordingly, any patient with these mutations would not be expected to display the target p53₂₆₄₋₂₇₂ epitope. Furthermore, there have been difficulties in generating, or even detecting [53], cytotoxic T cells to the p53₂₆₄₋₂₇₂ epitope in patients with p53 accumulation. This has led to a hypothesis that the specific epitope may not always be properly processed or presented from mutant p53 in cancer patients[28].

Thus, there exists a need in the art to generate additional therapeutic agents (for example, TCRm antibodies) targeting MHC class I-presented p53-derived peptides. Specifically, those which target epitopes that are displayed by a larger group of patients than the p53₂₆₄₋₂₇₂ epitope are desirable; in particular one inclusive of patients exhibiting the common R196X and R213X mutations.

### SUMMARY OF THE INVENTION

The present invention is based on the identification and characterisation of antibodies that bind to the cell surface major histocompatibility (MHC) class !-presented peptide p53₆₅₋₇₃, derived from the human intracellular tumour suppressor protein p53.

In particular an antibody (herein designated T1-116C) recognising the p53₆₅₋₇₃ peptide presented by HLA-A*0201 (also referred to in the art as HLA-A2) is characterised as an example of the present invention. Said antibody can bind to the surface of HLA-A2⁺/p53⁺ tumour cell lines and engage immune effector functions including complement-dependent cytotoxicity (CDC), antibody dependent phagocytosis (ADCP) and antibody-dependent cellular cytotoxicity (ADCC), thus indicating its suitability for use in cancer therapy.

Surprisingly, while the present inventors generated multiple antibodies that were capable of recognising different HLA-A2/p53 epitopes on the surface of target peptide-pulsed T2 lymphoblast cells, only an antibody recognising the P53₆₅₋₇₃ peptide (T1-116C) also had the desirable property of effectively recognising HLA-A2⁺/p53⁺ cancer cells.

The present invention is furthermore advantageous as an N-terminal p53-derived HLA-A*0201-presented epitope is recognised (residues 65-73). Thus, the invention may provide therapeutic benefit in patients exhibiting common mutations which lead to premature termination of p53 translation (R196X and R213X). This is in contrast to previous TCRm antibodies targeting HLA-A2-presented p53 epitopes (for example that disclosed in WO 2005/172160), where an epitope that is C-terminal to positions 196 and 213 is targeted (p53₂₆₄₋₂₇₂). Said epitope would not be presented by patients exhibiting the above mutations, thereby having no therapeutic utility in this patient subset.

It has been realised by the present inventors that the invention furthermore has utility as a companion diagnostic marker for cellular p53₆₅₋₇₃ presentation by HLA-A2*0201, in addition to its therapeutic applications (“theragnostic”—as known in the art).

According to a first aspect, the present invention provides an antibody which binds to human p53 tumour suppressor protein residues 65-73 (human p53₆₅₋₇₃), as shown in SEQ ID NO: 1, when presented by the MHC class I protein Human Leukocyte Antigen-A*0201 (HLA-A*0201).

According to a second aspect, the present invention provides a pharmaceutical composition comprising an antibody as defined herein, or the means for its expression, and a pharmaceutically acceptable diluent, excipient and/or adjuvant; optionally together with at least one additional therapeutic agent.

According to a third aspect, the present invention provides an antibody, or the means for its expression, or a pharmaceutical composition as defined herein, for use in therapy.

According to a fourth aspect, the present invention provides a hybridoma comprising and/or secreting an antibody as defined herein.

According to a fifth aspect of the present invention, there is a T-cell expressing a chimeric receptor comprising, in the extracellular domain thereof, an antibody as defined above as a single chain variable fragment.

According to a sixth aspect, the present invention provides a cell or cell line expressing an antibody as defined herein in recombinant form.

According to a seventh aspect, the present invention provides a recombinant expression vector, capable of expressing an antibody as defined herein.

According to an eighth aspect, the present invention provides the use of an antibody as defined herein, in an in vitro method for determining the level of cellular antigen presentation of human p53₆₅₋₇₃ by HLA-A*0201.

According to a ninth aspect, the present invention provides an in vitro diagnostic test for determining the suitability of a subject having a tumour/cancer to undergo immunotherapy; comprising contacting one or more cells obtained from the subject with an antibody as defined herein, and determining the presence, absence or level of binding of said antibody to the surface of said one or more cells; wherein

(1) said one or more cells comprise tumour cells; and wherein cell surface binding is a positive indication of the suitability of the subject to undergo said immunotherapy; or
(2) said one or more cells comprise non-malignant cells, and wherein cell surface binding is a negative indication of the suitability of the subject to undergo said immunotherapy;
wherein said immunotherapy is to be specific for human p53₆₅₋₇₃ presented by HLA-A*0201.

### BRIEF DESCRIPTION OF THE FIGURES

FIG. 1 shows the design and production of chimeric tetramers. Design of chimeric MHC class I tetramers showing replacement of the wild type human α3 domain and β2 microglobulin (β2m) on the left with their murine BALB/c counterparts, shown with a dashed line on the right (A). Substitution with murine β2m caused low refolding efficiency and hence low yields of HLA-A2/β2m/peptide complexed monomer, as shown in the FPLC chromatogram with the bold line (mβ2m-wt)(B). Structural analysis of the human and murine MHC-I molecules subsequently identified significant differences between the MHC-I interfaces in the human and murine β2m molecules. After identifying the amino acids responsible for these interactions, we mutated the relevant amino acids in the murine β2m protein to those of the human orthologue. This re-engineered murine β2m significantly increased monomer refolding efficiency (indicated with an asterisk in the peak shown with the dashed line). Chimeric tetramers retained their antigen specificity as shown by staining of an Influenza A virus (flu) M1 epitope GILGFVFTL-specific T-cell line (C). Immunisation with chimeric tetramers reduced the production of antibodies against the murine α3 domain and β2m domain but did not increase the frequency of TCRm antibody detection based on 2 fusions with wild type tetramers and 2 fusions with chimeric tetramers with the p53₆₅₋₇₃ peptide (D).

FIG. 2 illustrates examples of TCRm antibody reactivity against target (p53 peptide containing) versus control (flu peptide containing) tetramers by ELISA assay. Culture supernatants from late stage hybridoma colonies, or cloned hybridoma cell lines, were tested against their target p53 tetramer or control flu tetramer by ELISA, to determine their specificity. The OD reading at 450 nm from each sample was normalised against that of the positive control on the same plate for semi-quantitative comparison across experiments. Target 1 (T1) designation refers to p53 peptide 1 p53₆₅₋₇₃, and target 2 (T2) refers to p53 peptide 2, p53₁₈₇₋₁₉₇. The T1-29B antibody lacked sufficient specificity, despite preferentially binding the target peptide. The T2-108A antibody clones exhibited reduced specificity compared to that observed in early testing and the T2-2B clone lost target reactivity.

FIG. 3 shows that six p53 TCRm antibodies preferentially recognised T2 cells pulsed with the immunising p53 peptide and not an unrelated peptide. Human lymphoblastoid T2 cells that naturally express low levels of HLA-A2 on the cell surface were pulsed with (100 μM) of the appropriate immunising p53 peptide or a control peptide (GILGFVFTL) derived from Influenza A virus (flu). The six TCRm mAbs were each used to stain peptide pulsed T2 cells to detect the increased level of cell surface target peptide-HLA-A2 complex expression. Staining with the BB7.2 antibody (against HLA-A2) confirmed that both the p53 and flu peptides increased cell surface HLA-A2 expression. Only T2 cells pulsed with the immunising peptide from p53 stained strongly with the TCRm antibodies. Only T2-108A showed some binding of T2 cells pulsed with the flu-derived peptide.

FIG. 4 shows that the T2 cell staining with TCRm antibodies correlates with concentration of target peptide used for T2 pulsing and HLA-A2 mAb (BB7.2) staining. (A) T2 cells were pulsed with immunising peptide or a control peptide (derived from influenza A virus M1 protein, staining not shown) at the indicated concentrations overnight, then stained with the indicated TCRm mAbs (10 μg/ml). BB7.2 mAb (10 μg/ml) was used to stain each sample simultaneously. (B) Staining comparison shown as the ratios of TCRm mAb mean fluorescence intensity (MFI) against that of BB7.2 staining of the same sample (bottom panels). HLA-A2 expression is shown as BB7.2 staining (top panels). No staining of a control flu peptide-pulsed T2 cells was observed. The ability of p53 TCRm mAbs to bind the MHC class I presented target peptide in T2 cells was determined by the concentration of target peptide used to pulse the cells. T1-116C was the only antibody unable to markedly bind T2 cells pulsed with 500 nM of its p53 target peptide.

FIG. 5 illustrates the detection of p53 expression in cancer cell lines. (A) Three different commercial antibodies were used to detect p53 protein expression, p53 DO-7 (epitope amino acids 1-45), p53 pAB1801 (epitope amino acids 32-79) and p53 DO-1 (epitope amino acids 11-25). The TP53 mutation status is indicated as + for mutated, − for wild type, and a ? where the status is unknown. The HLA-A2 status and T1-116C binding are also indicated as either positive (+) or negative (−). TP53 transcript expression was determined using two independent Taqman probes targeting the indicated coding exons. Expression was normalised to B2M, 18S RNA and HPRT1 and expressed relative to expression in Granta-519.

FIG. 6 shows p53 TCRm antibody surface staining of cancer cell lines. Cancer cell lines derived from different tissues were stained with p53 TCRm antibodies (10 μg/ml purified antibody, with the exception of T1-116C data for 143B, SW480, AU565 and Hs-695T which were stained using hybridoma supernatant) and detected by anti-mouse-APC secondary antibody, followed by flow cytometry analysis. Secondary antibody-only staining was used as a negative control. T1-116C (p53₆₅₋₇₃) was the most effective TCRm antibody for immunolabelling cancer cells. Diffuse large B-cell lymphoma (DLBCL), chronic lymphocytic leukaemia (CLL).

FIG. 7 shows that the T1-116C mAb does not generally label normal HLA-A2⁺ PMBCs. (A) T1-116C hybridoma supernatant was used to stain PBMC buffy coat samples purified from 14 HLA-A2⁺ blood donors. Only one sample (Buf21) exhibited any T1-116C labelling. (B) The binding to sample Buf21 was confirmed by staining with purified T1-116C antibody (10 μg/ml). However, the PBMC sample exhibited an abnormal expansion of the granulocyte population, when compared with other normal samples (e.g. Buf10). (C) Illustrates TP53 transcript expression in the normal PBMC samples, determined using two independent Taqman probes targeting the indicated coding exons. Expression was normalised to B2M, 18S RNA and HPRT1 and expressed relative to a cancer cell line, Granta 519.

FIG. 8 shows immunocytochemical (ICC) labelling with the T1-116C antibody (murine IgG2a isotype, 10 μg/ml). (A) Acetone fixed cytospins of T2 cells, before and after being pulsed with the p53₆₅₋₇₃ peptide or a flu control peptide, were stained with antibody BB7.2 to illustrate the upregulation of HLA-A2 protein expression when T2 cells are pulsed with either peptide. T1-116C labelling was only observed when the T2 cells were pulsed with the target p53₆₅₋₇₃ peptide, illustrating the specificity of the ICC labelling. (B) Illustrates FACS staining of the same cell populations used for ICC using T1-116C and BB7.2 showing specificity of T1-116C labelling for T2 cells pulsed with the p53 peptide. Antibody binding was detected with anti-mouse-APC antibody, and secondary antibody-only staining was used as a negative control (grey peak).

FIG. 9 shows immunocytochemical labelling of acetone-fixed cytospins. NCI-H1395 lung cancer cells were labelled with the T1-116C mlgG1 and T1-116C mlgG2a format antibodies (A). No staining was observed using an equivalent concentration of a mlgG1 isotype control or IgG2a mouse isotype control antibody. p53 antibody clone D01 detected weak nuclear p53 protein expression (consistent with its WT mutation status) in NCI-H1395 cells. NCI-H2087 lung cancer cells were only weakly labeled with T1-116C, despite high-level p53 protein expression. HLA-A2 expression was strongly detected by antibody BB7.2 in both cell lines. All primary antibodies were used at 10 μg/ml. Antibody binding was detected using the Dako EnVision™ HRP/DAB system.

FIG. 10 shows that peptide scanning to identify residues whose conservation in the p53₆₅₋₇₃ peptide is necessary to enable effective T1-116C binding. (A) Single amino acids in the wild type p53 peptide 1 (RMPEAAPPV) were each replaced with an alanine (top panel) or glycine (bottom panel) residue. The substituted peptides were synthesised and used to pulse T2 cells (peptide concentration 100 μM), which were subsequently stained with HLA-A2 mAb BB7.2 (to predict changes affecting HLA-A2 binding), or TCRm mAb T1-116C and analysed by flow cytometry. The data shown are combined from three independent biological replicates. A consensus binding sequence of RXPXXAPXV, where the amino acid indicated by X can be substituted for another without >50% reduction in T1-116C antibody binding, was derived from this analysis. (B) The arginine (R) residue, at position one, was absolutely required for effective (>50%) T1-116C binding.

FIG. 11 shows T2 assay screening of the potentially cross-reactive peptides for HLA-A2 presentation and T1-116C binding. The UniProtKB/Swiss-Prot protein database was scanned using the ScanProsite tool with the T1-116C consensus sequence RXPXXAPXV. Individual peptides matching the T1-116C consensus sequence were synthesised and used to pulse T2 cells, which were subsequently stained with HLA-A2 mAb BB7.2 (to confirm loss of antibody binding was not purely a consequence of the substituted peptide's inability to bind HLA-A2), and TCRm mAb T1-116C and analysed by FACS. SHAN1 is also known as SHANK1. Labelling on the left indicates whether the peptides are human specific, identical in human and mouse or present in the mouse antigen only. Combined data from three replicate experiments is illustrated.

FIG. 12 shows quantitative real time PCR analysis of transcript expression for SHANK1, BSN and UBR3 in normal human tissues. Clontech's Human MTC™ Panel I and II were assayed for transcript expression using Taqman probes corresponding to best transcript coverage and/or the exon encoding the crossreactive peptide. Expression was normalised to GAPDH and B2M and expressed relative to a cancer cell line (ACH-N for SHANK1 and MDA-MB-435 for UBR3 and BSN). The coding exon for the cross reactive peptides is exon 5 (Ex5) for BSN, Ex22 for SHANK1 and UBR3.

FIG. 13 shows quantitative real time PCR analysis of transcript expression for UBR3, SHANK1 and BSN in cancer cell lines. Transcript expression was determined using two independent Taqman probes. Expression was normalised to TBP, 18S RNA and HPRT1 and expressed relative to expression in MDA-MB-435 or ACHN. The coding exon for the cross reactive peptides is exon 5 (Ex5) for BSN, Ex22 for SHANK1 and UBR3.

FIG. 14 shows Quartz crystal Microbalance (QCM) analysis to determine the affinity of T1-116C binding to its target p53₆₅₋₇₃ peptide/HLA*0201 complex. A polyclonal rabbit anti-human IgG antibody was immobilised on an Attana LNB Carboxy Sensor Chip via amine coupling. The T1-116C chimeric antibody (hlgG1) was captured and binding to the HLA-A2/p53 refolded protein was studied using an 8-point 1:2 serial dilution starting at a concentration of 50 μg/ml. Data curves were fitted to a ‘Simple 1:1 Binding Model’.

FIG. 15 shows that the T1-116C antibody can be internalised. Human B cell lymphoma OCI-Ly8 cells were stained with PE-conjugated T1-116C mAb (10 μg/ml) at 4° C. Aliquots of samples were fixed with 1% paraformaldehyde and the rest were incubated at 37° C. to allow antibody to internalise then harvested and stripped with a stripping buffer (150 mM NaCl, pH2.5) at the indicated time points before being fixed with 1% paraformaldehyde. Samples were analysed by FACS. Antibodies OKT3 and BB7.2 were used as negative and positive controls for internalisation.

FIG. 16 shows that the p53 TCRm T1-116C antibody can engage immune effector functions to achieve target cell killing of the B-cell lymphoma cell line OCI-Ly8. A human IgG1 chimeric form of T1-116C, at increasing concentrations (μg/ml), was used to induce human PBMC mediated ADCC (effector:target [E:T] ratio=50:1), mouse bone marrow-derived macrophage (BMDM)-mediated ADCP (E:T=5:1), or human serum complement mediated CDC against OCI-Ly8 cells. The anti-CD20 mAb Rituximab was used as a positive control. Herceptin was used as an isotype control antibody (Ctrl) at 10 μg/ml. Similar levels of ADCC and ADCP were observed against the B-cell lymphoma cell line OCI-Ly1 (data not shown).

FIG. 17 shows that the p53 TCRm T1-116C Ab prevents engraftment of a triple receptor negative breast cancer xenograft in vivo. 1×10⁷ human breast cancer MDA-MB-231 cells were injected subcutaneously into in BALB/c nu/nu mice (n=10 per group). T1-116C in two formats, a murine IgG2a isotype (mlgG2a) versus a human IgG1 isotype (hlgG1), or PBS carrier alone, was administered twice a week (10 mg/kg) starting from the time of tumour inoculation and tumour sizes were calculated as length×width×height×π/6. The mlgG2a T1-116C antibody, but not the hlgG1 T1-116C antibody significantly inhibited tumour growth (P<0.0001).

FIG. 18 shows that the p53 TCRm T1-116C Ab inhibits growth of an established triple receptor negative breast cancer xenograft in vivo. 1×10⁷ human breast cancer MDA-MB-231 cells were injected subcutaneously into Balb/c nu/nu mice (n=9 per group). Animals were regrouped at day 14 when tumours became palpable. T1-116C (murine IgG2a isotype), an IgG2a isotype control antibody against fluorescein, or PBS carrier alone, was then administered i.p. twice a week (10 mg/kg). Tumour sizes were calculated as (length×width×height)×π/6. The T1-116C antibody, but not the IgG2a control antibody or PBS, significantly inhibited tumour growth (P<0.0001).

FIG. 19 shows Quartz crystal Microbalance (QCM) analysis to determine the affinity of humanised and deimmunised T1-116C antibody variants for their target p53₆₅₋₇₃ peptide/HLA*0201 complex. A polyclonal rabbit anti-human IgG antibody was immobilised on an Attana LNB Carboxy Sensor Chip via amine coupling. The T1-116C humanised variant 1 or variant 2 was captured and binding to the HLA-A2/p53 refolded protein was studied using an 8-point 1:2 serial dilution starting at a concentration of 50 μg/ml. Data curves were fitted to a ‘Simple 1:1 Binding Model’.

FIG. 20 shows the four highest affinity humanised variants of T1-116C can recognise the p53₆₅₋₇₃ peptide/HLA*0201 complex binding on both T2 cells and a cancer cell line. (A) Illustrates T1-116C antibody staining of T2 cells pulsed with p53₆₅₋₇₃ peptide. A peptide (Flu) derived from Influenza M1 protein was used as a negative control. (B) Illustrates T1-116C staining of the NCI-H1395 lung cancer cell line. On both T2 cells and the cancer cell line T1-116C variants 1 and 2 (V1, V2) show labelling comparable to that of the chimeric antibody, while variants 3 and 4 (V3, V4) show less effective binding to the cell surface complex.

FIG. 21 shows T1-116C binding specificity in T2 pulsing assays. Individual amino acids in the p53₆₅₋₇₃ nonamer (RMPEAAPPV) were replaced with essential amino acids independently, generating 171 additional peptides for testing. T2 cells were pulsed with each of the peptides and stained with anti-HLA-A2 antibody BB7.2 (A) or T1-116C (B). Binding of peptide-presenting T2 cells by each antibody was compared with that of the original p53 peptide, and the bindings were categorised as enhanced (>100%), maintained (50-100%), reduced (25-50%), or diminished (<25%). Results in panel A confirmed that the anchor amino acid changes at position 2 and 9 have an important influence on peptide presentation by HLA-A2. Panel B indicates that arginine at position 1 is absolutely required for antibody binding, and other amino acids also contribute to the binding to lesser extends.

FIG. 22 shows alternative cancer relating peptides recognised by T1-116C. Peptide sequences confirming the binding consensus from FIG. 21 were retrieved from Immune Epitope Database (IEDB), or selected from literature as being important cancer targets with only one amino acid deviation from the consensus e.g. WT-1 and NY-ESO-1. Peptides were synthesised and tested in a T2 assay. An irrelevant peptide derived from influenza virus (GILGFTFVL) was used as a negative control. The murine T1-1160 antibody (mT1-1160) as well as the humanised antibodies (hT1-116CV1 and hT1-116CV2) were shown to be able to bind peptides derived from WT1, MG50, Tyrosinase, GP100 and NY-ESO-1. HLA-A2 expression was detected by the BB7.2 mAb.

FIG. 23 shows antibody radiolabelling. T1-1160-mlgG2a and an isotype control antibody were radiolabeled through pSCN-Bn-DTPA conjugation and 111In-chloride labelling. Radiochemical purity of the T1-1160 antibody was tested with instant thin layer chromatography (iTLC) before (A) and after (B) size exclusion purification. Similar results were obtained for the isotype control antibody (data not shown).

FIG. 24 shows a saturation binding assay. MDA-MB-231 breast cancer cells were incubated with 111In-T1-116C (humanised) antibody at various concentrations (2-400 nM) at 4° C. for 2 h, and cells were lysed and radioactivity was measured. An isotype control was tested in parallel. A saturation binding curve was fitted to the data using the GraphPad Prism software package to estimate the affinity (KD) and number of binding sites per cell (Bmax).

FIG. 25 shows antibody biodistribution in athymic mice bearing MDA-MB-231 (A) or MDA-MB-468 (B) xenografts. Radiolabelled T1-1160 (n=2) or an isotype control mAb (n=3) was injected into animals when tumour sizes reached 120 mm3, and radio-distribution was measured by SPECT/CT. SPECT/CT images represent individual mice at 24 h, 48 h and 72 h after antibody injection. The MDA-MB-231 tumours strongly labelled with T1-1160 in the far left panel are illustrated by white arrows.

FIG. 26 shows antibody biodistribution in athymic mice bearing MDA-MB-231 or MDA-MB-468 xenografts. Radiolabelled T1-116C (n=2) or an isotype control mAb (n=3) was injected in animals when tumour sizes reached 120mm3, and radio-distribution was measured by SPECT/CT. Ratio of antibody radio signals between tumour and heart (A), or tumour and liver (B) were calculated through VOI analysis on SPECT images. (C) Biodistribution after dissection. (***: P<0.001 by ANOVA). These data are derived from the same experiment as illustrated in FIG. 25.

FIG. 27 shows the design of the T1-116C Chimeric Antigen Receptor (CAR) in two single chain variable fragment (scFv) orientations, VHVL (left) and VLVH (right). T1-116C scFv sequences are followed by a spacer, a transmembrane domain, and a co-stimulatory domain, all derived from human CD28. A human CD3ξ signalling domain was added at the C-terminus to transduce activation signalling.

FIG. 28 shows the detection of T1-116C CAR cell surface expression and p53₆₅₋₇₃ tetramer binding. (A) HEK293T cells were transiently transfected with the two formats of T1-116C CAR expression constructs, VHVL and VLVH, and CAR receptor surface expression was detected by p53₆₅₋₇₃ tetramers. Control HLA-A2 tetramers loaded with Flu and p53₁₈₇₋₁₉₇ peptides, and streptavidin were stained simultaneously. Vector transfected HEK293T cells were used as a negative control. The mean fluorescence intensities (MFIs) of the tetramer staining were plotted in a bar graph (right) which shows significantly higher binding of p53₆₅₋₇₃ tetramer to T1-116C CAR-transfected cells than controls. (B) Streptavidin, non-specific tetramers (Flu or p53₁₈₇₋₁₉₇) and p53₆₅₋₇₃ tetramers were used to stain Jurkat cells stably expressing T1-116C-VLVH CAR receptor. The CAR-transduced Jurkat cells show binding to the p53₆₅₋₇₃ tetramer but not to the controls.

### DETAILED DESCRIPTION OF THE INVENTION

The present invention provides antibodies which bind to the p53₆₅₋₇₃ peptide derived from the tumour suppressor p53 protein, presented within the context of the human MHC haplotype HLA-A*0201 (an exemplary antibody according to the invention is herein designated T1-116C).

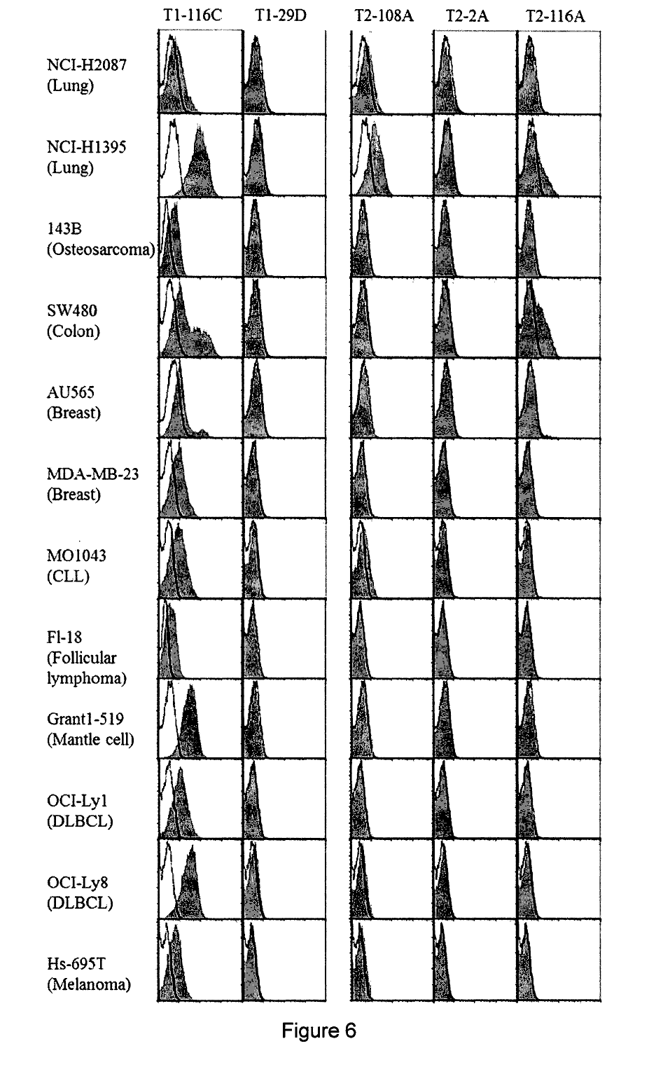

MF1 or BALB/c mice were immunised with human or chimeric (human/murine) tetramers containing peptides N-terminal to the common R196X and R231X mutations which causes premature termination of translation. After elimination of antibodies which cross-reacted with a tetramer containing a non-target peptide (SEQ ID NO:22), four hybridoma clones (T1-116C, T1-29D, T2-108A and T2-2A as denoted herein) were confirmed to exhibit specific recognition of their target P53 peptide, when presented on the cell surface of T2 cells in the context of HLA-A2*0201. However, only an antibody according to the invention (T1-116C) was able to immunolabel a wide range of cancer cell lines (FIG. 6; Table 3).

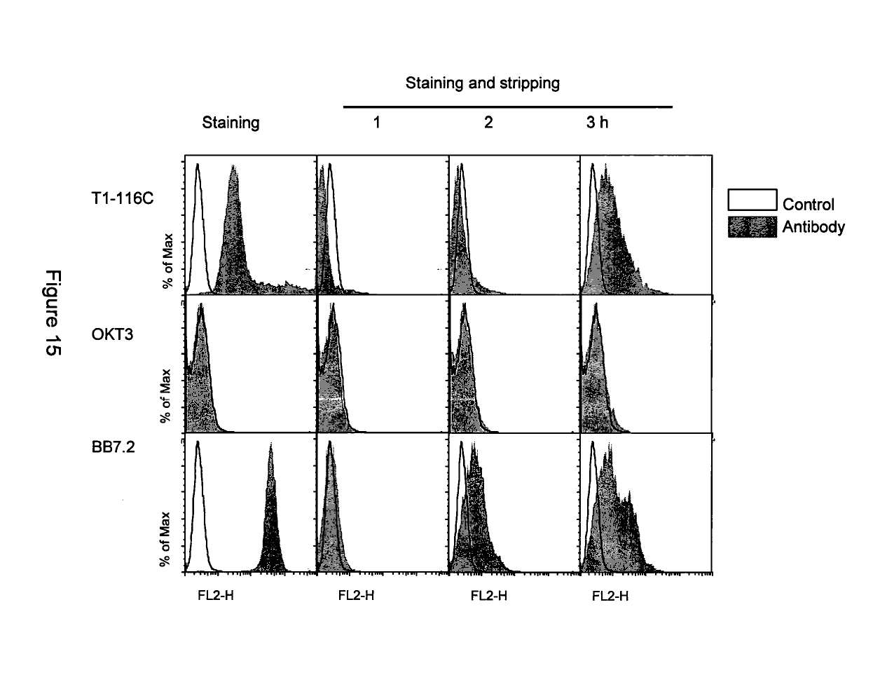

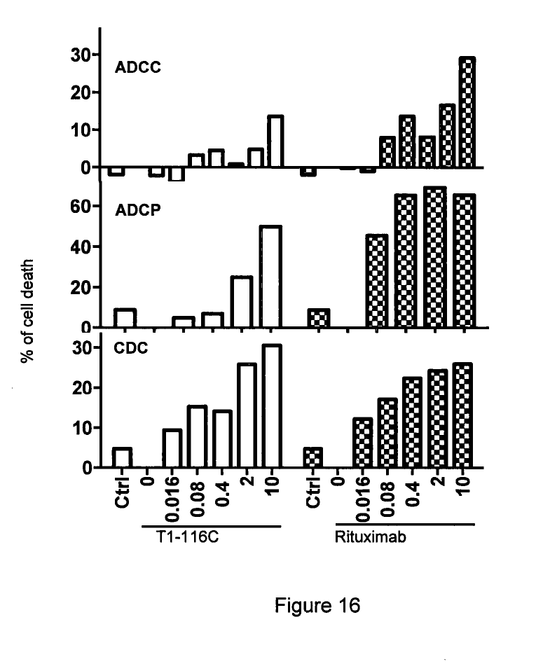

The present inventors investigated the ability of T1-116C to both be internalised by, and engage immune effector mechanisms against human lymphoma cells. T1-116C internalisation was detected within three hours of antibody incubation, indicating the potential for use of the antibody as a targeting moiety for antibody-drug conjugates (FIG. 15). Complement-dependent cytotoxicity (CDC), antibody dependent phagocytosis (ADCP) and antibody-dependent cellular cytotoxicity (ADCC) killing mediated by T1-116C was found, with comparable % cell kill to an isotype-matched Rituximab antibody (anti-CD20) (FIG. 16). This indicates the suitability of the antibody for cancer therapy.

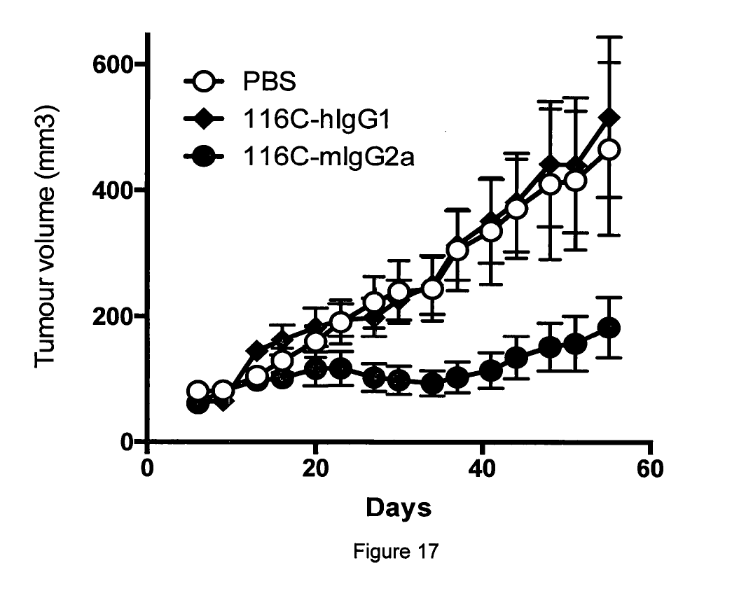

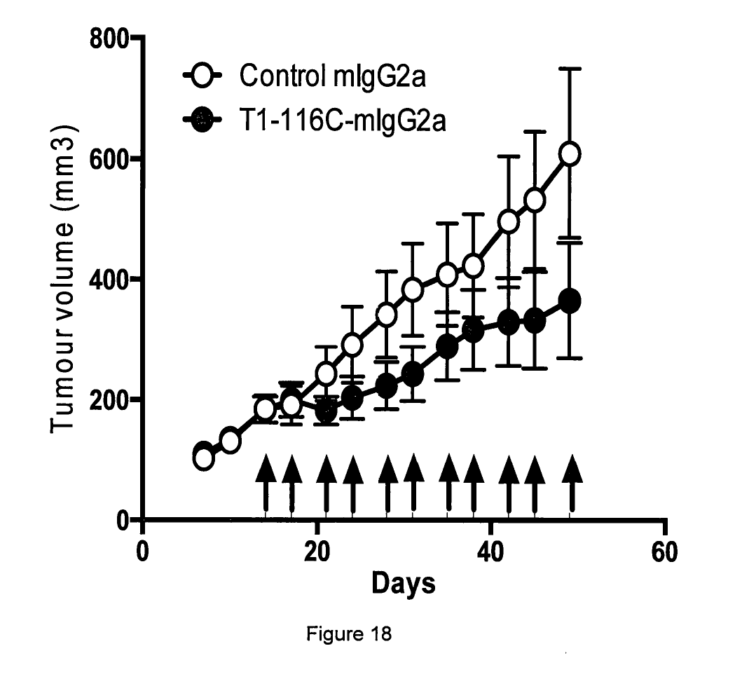

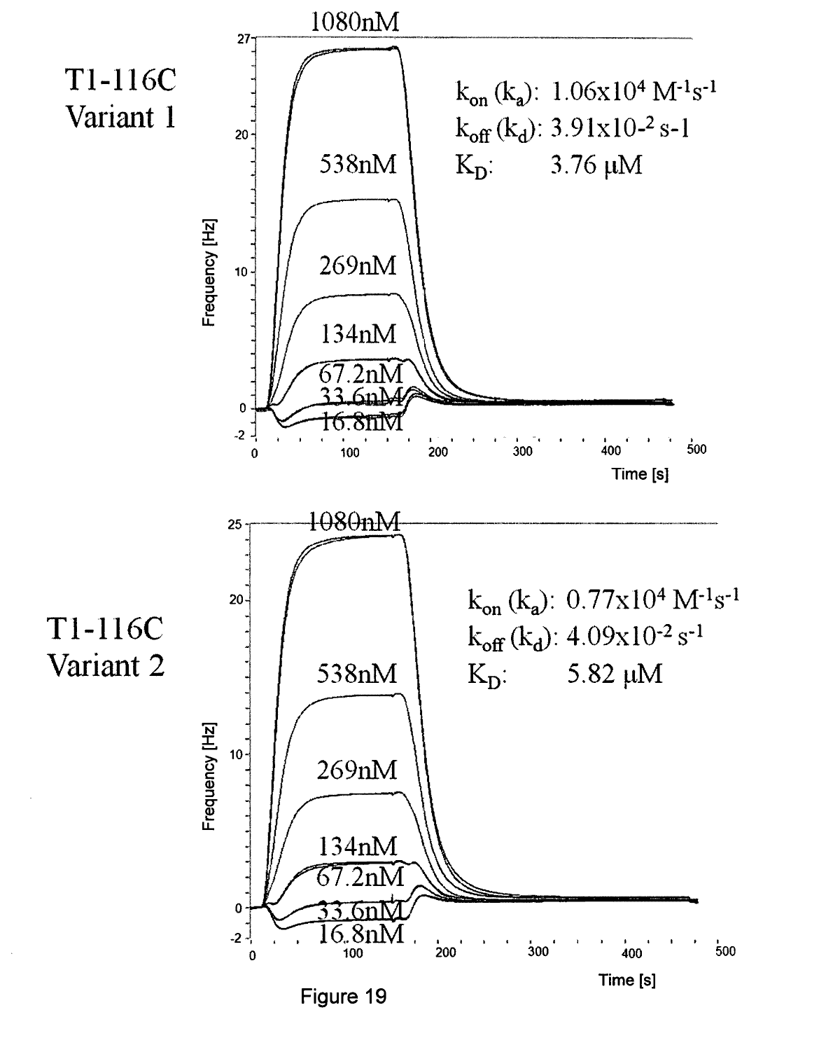

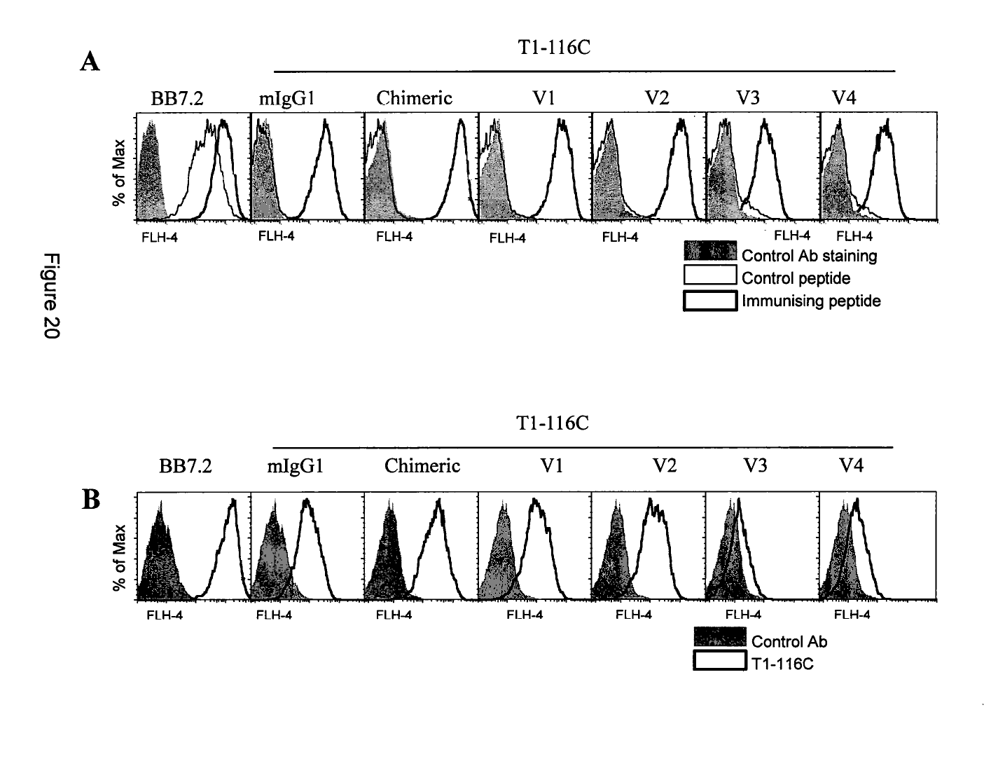

Furthermore, a mlgG2A format antibody was tested for its ability to prevent the engraftment of MBA-MB-231 tumours in vivo, which significantly reduced their growth rate (p<0.0001—FIGS. 17 and 18). Humanised and de-immunised variants of T1-116C were generated externally, with four able to bind to the target antigen. Variants 1 and 2 showed comparable affinity to the parental and chimeric antibody (Table 7; FIG. 19); effective binding by these variants was also found in the T2 assay (FIG. 20). Additional experimentation by the inventors is detailed in the Example.

The term “antibody” refers to an immunoglobulin which specifically recognises an epitope on a target, as determined by the binding characteristics of the immunoglobulin variable domains of the heavy and light chains (V_{H}s and V_{L}s), more specifically the complementarity-determining regions (CDRs). The terms “monoclonal antibody” and “antibody” are herein used interchangeably, unless otherwise stated. Many potential antibody forms are known in the art, and are included within the above definition; these may include, but are not limited to, a plurality of intact monoclonal antibodies or polyclonal mixtures comprising intact monoclonal antibodies, antibody fragments (for example F_{ab}, F_{ab}^{′}, F(_{ab}′)₂ and Fᵥ fragments, linear antibodies, single chain antibodies and multispecific antibodies comprising antibody fragments), single chain variable fragments (scFᵥs), multispecific antibodies, chimeric antibodies, humanised antibodies and fusion proteins comprising the domains necessary for the recognition of a given epitope on a target. Antibodies may also be conjugated to various moieties for a therapeutic effect, including but not limited to drugs (especially cytotoxic drugs) and radioisotopes. An antibody may comprise γ, δ, α, μ and ε type heavy chain constant domains, wherein an antibody comprising said domains is designated the class IgG, IgD, IgA, IgM or IgE respectively. Classes may be further divided into subclasses according to variations in the sequence of the heavy chain constant domain (for example IgG1-4). Light chains are designated either K or A class, depending on the identity of the constant region.

The term “variable region” of an antibody refers to the variable region of the antibody light chain or the variable region of the antibody heavy chain, either alone or in combination. The variable regions of the heavy and light chains each consist of four framework regions (FR) connected by three complementarity determining regions (CDRs) also known as “hypervariable regions”. The CDRs in each chain are held together in close proximity by the FRs and, with the CDRs from the other chain, contribute to the formation of the antigen-binding site of the antibody. There are at least two techniques for determining CDRs: (1) an approach based on cross-species sequence variability [69]; and (2) an approach based on crystallographic studies of antigen-antibody complexes [70]. In addition, combinations of these two approaches are sometimes used in the art to determine CDRs.

The term “monoclonal antibodies” refers to a homogenous population of antibodies (including the forms previously described, for example antibody fragments), which recognise a single epitope on a target. This is in contrast to polyclonal antibodies that typically include a mixture of different antibodies directed against different antigenic determinants. Furthermore, “monoclonal antibodies” includes such antibodies generated by any number of techniques including, but not limited to, hybridoma production, phage selection, recombinant expression, and transgenic animals.

The term “humanised antibody” refers to forms of non-human (e.g., murine) antibodies that are specific immunoglobulin chains, chimeric immunoglobulins, or fragments thereof that contain minimal non-human (e.g., murine) sequences.

The term “de-immunised antibody” refers to antibodies that are specific immunoglobulin chains, chimeric immunoglobulins, or fragments thereof that contain minimal sequences encoding potential T-cell epitopes.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammal (e.g., mouse, rat, rabbit, etc.) with the desired specificity, affinity, and/or capability while the constant regions are homologous to the sequences in antibodies derived from another species (usually human) to avoid eliciting an immune response in that species.

According to the present invention, the target is a complex formed by non-covalent interaction between a Major Histocompatability Complex (MHC) class I protein (HLA-A*0201) and a small, linear peptide, human p53₆₅₋₇₃ having the sequence RMPEAAPPV (SEQ ID NO: 1). As such, the antibody of the present invention interacts with a composite of specific residues from the linear peptide that is presented by the MHC class I protein, and residues of the MHC class I protein itself (notably, residues from the a helices of the α₁ and α₂ domains which create the binding site for, and flank the linear peptide when presented).

The term “binds to”, in the context of antibody-target interactions, refers to an interaction wherein the antibody and target associate more frequently or rapidly, or with greater duration or affinity, or with any combination of the above, than when either antibody or target is substituted for an alternative substance, for example an unrelated protein. Generally, but not necessarily, reference to binding means specific recognition. Techniques known in the art for determining the specific recognition of a target by a monoclonal antibody, or lack thereof, include but are not limited to, FACS analysis, immunocytochemical staining, immunohistochemistry, immunofluorescence, western blotting/dot blotting, ELISA, affinity chromatography. By way of example and not limitation, specific recognition, or lack thereof, may be determined by comparative analysis with a control comprising the use of an antibody which is known in the art to specifically recognise said target and/or a control comprising the absence of, or minimal, specific recognition of said target (for example wherein the control comprises the use of a non-specific antibody). Said comparative analysis may be either qualitative or quantitative.

The terms “polypeptide”, “peptide” and “protein” are used interchangeably herein and have their conventional meaning in the art. The terms “amino acid” and “residue” are herein used interchangeably and have their conventional meanings in the art. Specific amino acids are herein referred to by their conventional one and three letter codes. Furthermore, human p53 residues are numbered in accordance with Uniprot entry P04637 (http://www.uniprot.org). As used herein, when reference is made to “the [amino acid] sequence of” a peptide, protein or part thereof (for example, p53₆₅₋₇₃), this includes, but is not limited to, the sequence of amino acids in question having been derived from a source other than said peptide, protein or part thereof. For example, “the [amino acid] sequence of p53₆₅₋₇₃” includes said sequence when generated by intracellular proteolytic processing of human p53 tumour suppressor protein. However, the term also encompasses, for example, a peptide of the same sequence which has been obtained via in vitro synthesis (which has not been derived from full-length p53). Such a peptide may be presented by HLA-A*0201 in a cell line (for example, T2 lymphoblast cells) in vitro after pulsing of the cells with the peptide (see Example).

The terms “identical” or percent “identity”, in the context of two or more polypeptides, refer to two or more sequences or sub-sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software are known in the art that may be used to obtain alignments of amino acid or nucleotide sequences. These include, but are not limited to, BLAST and ALIGN.

The terms “cancer” and “cancerous” refer to or describe the physiological condition in mammals in which a population of cells are characterised by unregulated cell growth, proliferation and/or survival.

The terms “tumour” and “cancer” refer to any mass of tissue that results from excessive cell growth, proliferation and/or survival, and that is of a malignant nature, including pre-cancerous lesions.

The term “subject” refers to any animal (e.g., a mammal), including, but not limited to, humans, non-human primates, canines, felines, rodents, and the like, which is to be the recipient of a particular therapy. The terms “subject” and “patient” are used interchangeably herein in reference to a human subject, which is especially envisaged according to the present invention.

The phrase “pharmaceutically acceptable excipient, carrier or adjuvant” refers to an excipient, carrier or adjuvant that can be administered to a subject, together with at least one antibody of the present disclosure, and which does not destroy the pharmacological and/or biological activity thereof and is nontoxic when administered in doses sufficient to deliver a therapeutic amount of the antibody.

The phrase “therapeutically effective amount” refers to an amount of an agent effective to “treat” a disease or disorder in a subject or elicit an effect on one or more cells in vitro. In the case of a subject having a tumour/cancer, the therapeutically effective amount of the agent (e.g., an antibody) can reduce the number of cancer cells; reduce the tumour size; inhibit and/or stop cancer cell infiltration into peripheral organs including, for example, the spread of cancer into soft tissue and bone; inhibit and/or stop tumour metastasis; inhibit and/or stop tumour growth; relieve to some extent one or more of the symptoms associated with the cancer; reduce morbidity and mortality; improve quality of life; decrease tumourigenicity, tumourigenic frequency, or tumourigenic capacity of a tumour; reduce the number or frequency of cancer stem cells in a tumour; differentiate tumourigenic cells to a non-tumourigenic state; or a combination of such effects. To the extent the agent prevents growth and/or kills existing cancer cells, it can be referred to as cytostatic and/or cytotoxic. In the case of an effect on one or more cells in vitro, a therapeutically effective amount of agent may inhibit tumour cell growth when an immortalized cell line or a cancer cell line, or tumour cells isolated from a patient sample such as, for example, a tissue biopsy, pleural effusion, bone marrow aspirate or blood sample is/are cultured in a medium containing said agent.

The term “therapy” encompasses both “treatment” and “prevention”; thereby referring to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of therapy include those already with the disorder; those prone to have the disorder; and those in whom the disorder is to be prevented. In certain embodiments, a subject is successfully “treated” for a tumour/cancer according to the present invention if the subject shows one or more of the following: a reduction in the number of, or complete absence of, cancer cells; a reduction in the tumour size; inhibition of, or an absence of, cancer cell infiltration into peripheral organs including, for example, the spread of cancer into soft tissue and bone; inhibition of, or an absence of, tumour metastasis; inhibition of, or an absence of, tumour growth; relief of one or more symptoms associated with the specific cancer; reduced morbidity and mortality; improvement in quality of life; reduction in tumourigenicity, tumourigenic frequency, or tumourigenic capacity of a tumour; reduction in the number or frequency of cancer stem cells in a tumour; differentiation of tumourigenic cells to a non-tumourigenic state; or some combination of effects.

According to a first aspect, the present invention provides an antibody which binds to human p53 tumour suppressor protein residues 65-73 (human p53₆₅₋₇₃), as shown in SEQ ID NO: 1, when presented by the MHC class I protein Human Leukocyte Antigen-A*0201 (HLA-A*0201). As detailed above, the target will inherently further comprise residues contributed by HLA-A*0201 (notably those of the α₁ and α₂ domains thereof).

The antibody preferably exhibits greater cell surface binding to T2 lymphoblast cells (ATCC CRL-1992) presenting the sequence of human p53₆₅₋₇₃ by HLA-A*0201, than a non-specific peptide. More preferably, said non-specific peptide consists of the amino acid sequence GILGFVFTL derived from influenza A virus (SEQ ID NO: 22). For the avoidance of doubt, T2 cells presenting either the sequence of human p53₆₅₋₇₃ or the non-specific peptide by HLA-A*0201 are to be prepared in accordance with the methods detailed in the Example (T2 cell binding assay). Analysis of cell surface binding (or lack thereof) may be performed via fluorescence-activated cell sorting (FACS) or suitable derivative methods thereof known to the skilled person, or via immunocytochemistry or suitable derivative methods thereof known to the skilled person; preferably in accordance with the methods detailed in the Example (FACS Analysis and Immunocytochemistry).

Comparatively greater cell surface binding to T2 cells presenting the sequence of human p53₆₅₋₇₃ by HLA-A*0201, than a non-specific peptide, may be confirmed by either method. It is especially preferred that said analysis further comprises incubation of T2 cells with a positive control antibody for HLA-A*0201, for example BB7.2 (as detailed in the Example), to confirm the presence of cell-surface HLA-A*0201.

The antibody preferably binds to the cell surface of an HLA-A2⁺/p53⁺ cancer cell line; more preferably any cell line selected from the group consisting of NCI-H2087 (lung) (ATCC CRL-5922), NCI-H1395 (lung) (ATCC CRL-5868), Hs-695T (melanoma) (ATCC HTB-137), 143B (osteosarcoma) (ATCC CRL-8303), SW480 (colon) (ATCC CCL-228), AU565 (breast) (ATCC CRL-2351), MDA-MB-231 (breast) (ATCC HTB-26), MO-1043 (chronic lymphocytic leukaemia) (see [78]), FL-18 (follicular lymphoma) (see [79]), Granta-519 (mast cell leukaemia) (DSMZ ACC 342), OCI-Ly1 (diffuse large B-cell lymphoma) (DSMZ ACC 722) and OCI-Ly8 (diffuse large B-cell lymphoma) (see [80]); even more preferably any cell line selected from the group consisting of NCI-H2087 (lung), NCI-H1395 (lung), Hs-695T (melanoma), 143B (osteosarcoma), SW480 (colon), AU565 (breast), MDA-MB-231 (breast), Granta-519 (mast cell leukaemia) and OCI-Ly1 (diffuse large B-cell lymphoma). It is most preferred that the antibody binds to all of the above cell lines from either selection (above). Binding may be confirmed via FACS or suitable derivative methods thereof known to the skilled person. For the avoidance of doubt, this is to be performed in accordance with the methods detailed in the Example (FACS analysis). It is especially preferred that binding is confirmed relative to a negative control, non-specific, antibody; for example an anti-mouse-APC antibody (as detailed in the Example), for which no cell surface binding would be observed.

The antibody preferably has a dissociation constant (K_{D}), with respect to the sequence of human p53₆₅₋₇₃ when presented by HLA-A*0201, of 200 μM or less, preferably 150 μM or less, preferably 0.005-110 μM, preferably 0.01-110 μM, more preferably 0.8-30 μM, even more preferably 0.90-5.5 μM; wherein said dissociation constant has been determined by a quartz crystal microbalance assay. For the avoidance of doubt, this is to be performed in accordance with the methods detailed in the Example (Quartz crystal Microbalance (QCM) analysis). An exemplary antibody according to the invention (T1-116C) demonstrates a K_{D} (as defined above) of 0.977 μM. A chimeric human IgG1 format T1-116C antibody demonstrates a K_{D} of 1.25-1.76 μM, while humanised variants 1-4 demonstrate K_{D} of 3.76 μM, 5.32 μM, 29.30 μM or 108 μM, respectively.

It is preferred that the antibody, when applied in vitro to the surface of human B cell lymphoma cells at 10 μg/ml and subsequently incubated at 37° C., is internalised. More preferably, the cells are of the cell line OCI-Ly8. For the avoidance of doubt, internalisation is confirmed, in accordance with the methods detailed in the Example (Antibody internalisation assay). It is especially preferred that internalisation is confirmed relative to a positive or negative control antibody for such internalisation (for example, BB7.2 (from hybridoma ATCC HB-82) or OKT3 (from hybridoma ATCC CRL-8001), respectively, as detailed in the Example); most preferably both.

Further, the antibody is able to elicit antibody-dependent cellular phagocytosis (ADCP), antibody-dependent cellular cytotoxicity (ADCC) and complement-dependent cytotoxicity (CDC) immune effector mechanisms. Preferably, said activities are confirmed in vitro against a B-cell lymphoma cell line (more preferably OCI-Ly8) wherein antibody-induced ADCP is mediated by bone marrow-derived macrophages, ADCC is mediated by peripheral blood mononuclear cells, and CDC is mediated by serum comprising complement. For the avoidance of doubt, said activities may be confirmed in accordance with the methods detailed in the Example (Complement Dependent Cytotoxicity (CDC) Assay; Antibody Dependent Cellular Phagocytosis (ADCP) Assay; Antibody Dependent Cellular Cytotoxicity (ADCC) Assay). It is especially preferred that said activities are confirmed relative to a positive control antibody capable of eliciting said immune effector mechanisms, for example Rituximab when assaying against CD20+B cells (for example, OCI-Ly8 as detailed in the Example). ADCP by the antibody may be confirmed in accordance with the protocols described above by achieving at least 50%, preferably at least 60%, more preferably at least 70% of the % cell death achieved by Rituximab (both antibodies at 10 μg/ml) (see, for example, FIG. 16). ADCC by the antibody may be confirmed in accordance with the protocols described above by achieving at least 25%, preferably at least 30%, more preferably at least 35% of the % cell death achieved by Rituximab (both antibodies at 10 μg/ml) (see, for example, FIG. 16). CDC by the antibody may be confirmed in accordance with the protocols described above by achieving at least 70%, preferably at least 80%, more preferably at least 90% of the % cell death achieved by Rituximab (both antibodies at 10 μg/ml) (see, for example, FIG. 16).

An antibody of the invention may preferably comprise a CDR-L1 having the amino acid sequence of SEQ ID NO: 2; a CDR-L2 having the amino acid sequence of SEQ ID NO: 3; and a CDR-L3 having the amino acid sequence of SEQ ID NO: 4. In combination with the above light chain CDR sequences, the antibody may comprise:

(1) a CDR-H1 having the amino acid sequence of SEQ ID NO: 7; a CDR-H2 having the amino acid sequence of SEQ ID NO: 8; and a CDR-H3 having the amino acid sequence of SEQ ID NO: 9; or

(2) a CDR-H1 having the amino acid sequence of SEQ ID NO: 7; a CDR-H2 having the amino acid sequence of SEQ ID NO: 11; and a CDR-H3 having the amino acid sequence of SEQ ID NO: 9; or

(3) a CDR-H1 having the amino acid sequence of SEQ ID NO: 10; a CDR-H2 having the amino acid sequence of SEQ ID NO: 11; and a CDR-H3 having the amino acid sequence of SEQ ID NO: 9.

Combination (1) is characteristic of T1-116C wild type antibody, T1-116C humanised and deimmunised variant 1 and T1-116C humanised and deimmunised variant 2, combination (2) is characteristic of T1-116C humanised and deimmunised variant 3, and combination (3) is characteristic of humanised and deimmunised variant 4.

In alternative embodiments, the antibody may comprise variants of any of the above CDR L1-L3 and CDR H1-H3 combinations with (in increasing preference) no more than 4, 3, 2, or 1 substitutions, deletions and/or insertions (the total number of substitutions, deletions and insertions not exceeding 4, 3, 2 or 1); more preferably no more than 4, 3, 2, or 1 substitutions as the only variation; as compared to SEQ ID NO: 2, 3, 4, 7, 8, 10 and 11 where appropriate.

Preferably:
(i) the antibody of (1) comprises a light chain variable domain (V_{L}) having the sequence of SEQ ID NO: 12 and a heavy chain variable domain (V_{H}) having the sequence of SEQ ID NO: 17; or
(ii) the antibody of (1) comprises a V_{L} having the sequence of SEQ ID NO: 13 and a V_{H} having the sequence of SEQ ID NO: 18; or
(iii) the antibody of (1) comprises a V_{L} having the sequence of SEQ ID NO: 13 and a V_{H} having the sequence of SEQ ID NO: 19; or
(iv) the antibody of (2) comprises a V_{L} having the sequence of SEQ ID NO: 13 and a V_{H} having the sequence of SEQ ID NO: 20; or
(v) the antibody of (3) comprises a V_{L} having the sequence of SEQ ID NO: 13 and a V_{H} having the sequence of SEQ ID NO: 21.

Combination (i) is characteristic of T1-116C wild type antibody, combination (ii) is characteristic of T1-116C humanised and deimmunised variant 1, combination (iii) is characteristic of T1-116C humanised and deimmunised variant 2, combination (iv) is characteristic of T1-116C humanised and deimmunised variant 3 and combination (v) is characteristic of T1-116C humanised and deimmunised variant 4.

In alternative embodiments, the antibody may comprise variants of any of the above V_{L} and V_{H} combinations with (in increasing preference) at least 70%, at least 80%, at least 90%, or at least 95% sequence identity to SEQ ID NO: 12, 13, 17, 18, 19, 20 and 21 where appropriate. The percentage sequence identity may be measured using sequence comparison software or algorithms or by visual inspection. Various algorithms and software are known in the art that may be used to obtain alignments of amino acid sequences. These include, but are not limited to, BLAST and ALIGN.

The present invention further provides pharmaceutical compositions comprising one or more of the antibodies described herein. In certain embodiments, the pharmaceutical compositions further comprise a pharmaceutically acceptable vehicle or diluent, examples of which are known in the art. These pharmaceutical compositions find use in inhibiting tumour growth and treating cancer in a subject (e.g., a human patient).

In certain embodiments, compositions are prepared for storage and use by combining a purified antibody described herein with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propylparaben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG).

The pharmaceutical compositions can be administered in any conventional way. Administration can be by parenteral administration, more particularly by intravenous administration. However, other routes of administration are also envisaged. In certain embodiments, in addition to comprising an antibody as described herein, the pharmaceutical compositions of the present invention further comprise at least one additional therapeutic agent. In certain embodiments, the at least one additional therapeutic agent comprises 1, 2, 3, or more additional therapeutic agents.

The present invention also provides an antibody as described herein, or the means for its expression, or a pharmaceutical composition according as defined herein, for use in therapy. Said therapy is most preferably of a human subject having the haplotype HLA-A*0201, as determined by standard methods of genetic testing, available to the skilled person. An antibody of the invention may also be administered to a subject via gene therapy techniques (as an example of means for its expression), whereby subject patient is administered a polynucleotide that encodes the antibody. For example, a gene therapy vector, comprising a polynucleotide that encodes the antibody, may be administered, such that the antibody is expressed in vivo. Gene therapy techniques may also be used to introduce the polynucleotide encoding the antibody into T cells, ex vivo, which are then administered to a patient as an adoptive cell therapy. The delivery of an antibody by such suitable techniques will be apparent to the skilled person [71], [72].

The therapy is preferably that of a subject having a tumour/cancer. More preferably, the tumour/cancer is selected from the group consisting of lung cancer, melanoma, osteosarcoma, colon cancer, breast cancer, chronic lymphocytic leukaemia, follicular lymphoma, mast cell leukaemia, diffuse large B-cell lymphoma, prostate cancer, pancreatic cancer, ovarian cancer and mantle cell lymphoma. The above tumour cell types are known in the art to exhibit p53 overexpression, increased p53 peptide presentation and/or altered activity through mutation or other p53 pathway alterations, as compared to non-malignant cells of same tissue type;

in addition, immunolabelling of cell lines from a number of the above tumour types has been confirmed experimentally with an antibody according to the invention (T1-116C—see Example; Table 3; FIG. 6). Even more preferably, the tumour/cancer is selected from the group consisting of lung cancer, melanoma, osteosarcoma, colon cancer, breast cancer, chronic lymphocytic leukaemia, follicular lymphoma, mast cell leukaemia, diffuse large B-cell lymphoma, pancreatic cancer, and mantle cell lymphoma. Immunolabelling of cell lines from all of the above tumour types has been confirmed experimentally with an antibody according to the invention (T1-116C—see Example; Table 3; FIG. 6). In certain embodiments, at least one additional therapeutic agent can be administered prior to, concurrently with, and/or subsequently to, administration of antibody described herein during therapy (combination therapy).

In some embodiments, 1, 2, 3, or more additional therapeutic agents may be administered. Combination therapy with at least two therapeutic agents often involves agents that work by different mechanisms of action, although this is not required. Combination therapy using agents with different mechanisms of action may result in additive or synergistic effects. Combination therapy may allow for a lower dose of each agent than is used in monotherapy, thereby reducing toxic side effects. Combination therapy may decrease the likelihood that resistant cancer cells will develop. Combination therapy may allow for one therapeutic agent to be targeted to tumourigenic cancer stem cells, while a second therapeutic agent may be targeted to non-tumourigenic cancer cells.

An additional therapeutic agent may be administered in any order or concurrently with the antibody as described herein. In some embodiments, the antibody will be administered to patients that have previously undergone treatment with a second therapeutic agent. In certain other embodiments, the antibody and a second therapeutic agent will be administered substantially simultaneously or concurrently. For example, a subject may be given the antibody while undergoing a course of treatment with a second therapeutic agent (e.g., chemotherapy). In certain embodiments, the antibody will be administered within 1 year of the treatment with a second therapeutic agent. In certain alternative embodiments, the antibody will be administered within 10, 8, 6, 4, or 2 months of any treatment with a second therapeutic agent. In certain other embodiments, the antibody will be administered within 4, 3, 2, or 1 weeks of any treatment with a second therapeutic agent. In some embodiments antibody will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an antimetabolite, an antimitotic, a topoisomerase inhibitor, or an angiogenesis inhibitor.

Therapeutic agents that may be administered in combination with the antibody as described herein include chemotherapeutic agents. Thus, in some embodiments, the therapy involves the combined administration of the antibody and a chemotherapeutic agent or cocktail of multiple different chemotherapeutic agents. Treatment with the antibody can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethane; vindesine; dacarbazine; mannomustine; mitobronitol; mitolactol; pipobroman; gacytosine; arabinoside (“Ara-C”); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11, topoisomerase inhibitor RFS 2000; difluoromethylomithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumours such as antiestrogens including for example tamoxifen, raloxifene, aromatase inhibiting 4(5)-imidazoles, 4-hydroxytamoxifen, trioxifene, keoxifene, LY1170 18, onapristone, and toremifene (Fareston); and antiandrogens such as flutamide, nilutamide, bicalutamide, leuprolide, and goserelin; and pharmaceutically acceptable salts, acids or derivatives of any of the above.

The chemotherapeutic agent may be a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In certain embodiments, the second therapeutic agent is irinotecan.

The chemotherapeutic agent may also be an anti-metabolite. An anti-metabolite is a chemical with a structure that is similar to a metabolite required for normal biochemical reactions, yet different enough to interfere with one or more normal functions of cells, such as cell division. Antimetabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the second therapeutic agent is gemcitabine.

The chemotherapeutic agent may also be an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or PlkI. In certain embodiments, where the chemotherapeutic agent administered in combination with the antibody is an anti-mitotic agent, the cancer or tumour being treated is breast cancer or a breast tumour.

Combination therapy is also envisaged, and may involve the combined administration of the antibody as described herein and radiotherapy. Administration of the antibody as described herein can occur prior to, concurrently with, or subsequent to administration of radiotherapy by the skilled medical practitioner.

It is also envisaged that a second therapeutic agent may comprise a further antibody. Thus, treatment can involve the combined administration an antibody of the present invention with other antibodies against additional tumour-associated antigens including, but not limited to, antibodies that bind to EGFR, ErbB2, HER2, DLL4, Notch, PD-1, PD-1L, CTLA-4 and/or VEGF. In certain embodiments, a second therapeutic agent is an antibody that is an angiogenesis inhibitor (e.g., an anti-VEGF antibody). In certain embodiments, a second therapeutic agent is bevacizumab (AVASTIN), trastuzumab (HERCEPTIN), panitumumab (VECTIBIX), or cetuximab (ERBITUX). In certain embodiments a second therapeutic agent is an antibody that inhibits an immune checkpoint receptor ligand interaction (e.g. PD-1, PD-1L, CTLA-4 and others) ipilimumab (YERVOY), pembrolizumab (KEYTRUDA), nivolumab (OPDIVO). Combined administration can include co-administration, either in a single pharmaceutical formulation or using separate formulations, or consecutive administration in either order but generally within a time period such that all active agents can exert their biological activities simultaneously.

Combination therapy with the antibodies described herein can include treatment with one or more cytokines (e.g., lymphokines, interleukins, tumour necrosis factors, and/or growth factors)

Therapy with the antibodies described herein can be accompanied by surgical removal of tumours, cancer cells or any other therapy deemed necessary by a treating physician.

The antibody described herein may also be delivered in conjunction with an oncolytic virus in the therapy of a tumour/cancer. This may improve the bystander killing effect of such therapy.

The antibody described herein may be used as a delivery means for a drug (preferably, a cytotoxic drug), radioisotope, nanoparticle or further antibody to the cells of the tumour/cancer as described above; by way of standard conjugation and/or labelling methods available in the art to the skilled person. By delivery means, it is meant that the antibody (according to the invention) acts as a targeting moiety to localise the above agents to the cells of the tumour/cancer, through specific recognition of p53₆₅₋₇₃ in the context of HLA-A*0201. Wherein the antibody according to the invention is conjugated to a further antibody (as a bispecific antibody construct), preferred further antibodies include, but are not limited to, anti-CD3 antibodies (for example as found in BITE® bispecific T cell engagers—available from Amgen Oncology); checkpoint inhibitors (including inter alia anti-PD-1, PD-1L and CTLA-4 antibodies, for example ipilimumab (YERVOY), pembrolizumab (KEYTRUDA), nivolumab (OPDIVO)); and angiogenesis inhibitors (for example, anti-VEGF antibodies). In such bi-specific antibody constructs, both antibodies are both typically, but not necessarily, in the form of single chain variable fragments. Wherein the antibody is conjugated to a radiolabel, positron emission tomography (PET) may be used to determine tumour or off-target binding. Furthermore, when the antibody is conjugated to a nanoparticle, preferably iron nanoparticle, magnetic resonance imaging (MRI) may be used to a similar effect. It is envisaged that an initial sub-therapeutic dose to the subject may be used to set dosing thresholds and indicate their response, prior to the administration of the therapeutically-effective dose.

The antibody described herein may also be used as a delivery means for an immune effector cell to the cells of the tumour/cancer, wherein the immune effector cell expresses a chimeric receptor comprising the antibody as a single chain variable fragment. The antibody will be found in the extracellular domain of the receptor, allowing specific recognition of p53₆₅₋₇₃ in the context of HLA-A*0201. Preferably, the immune effector cell is a T cell. Preferably, the receptor comprises the antibody as described above, linked to one or more intracellular co-stimulatory signalling domains (typically either 1, 2 or 3 domains), such as the Fc receptor γ chain, to activate the immune effector, preferably T cell. Chimeric antigen receptor T cells (CAR T-cells) are known in the art [73-75], and may be produced by standard methods known to the skilled person. Such methods typically comprise the introduction to the cell of a chimeric gene incorporating the antibody as a single chain variable fragment, linked to the one or more signalling domains.

The present invention furthermore provides a method of treating or preventing a tumour/cancer in a subject in need of such treatment or prevention, by administering a therapeutically-effective amount of an antibody as defined herein to the subject. Said method has the same optional and preferred features as described above.

The present invention also provides a hybridoma comprising and/or secreting an antibody according as described herein. Said hybridoma may be obtained by standard fusion protocols [67] after murine immunisation according to the methods of the Example (Cell culture; Generation of HLA-A2/p53 tetramer and chimeric tetramer; Generation of anti-p53 TCR monoclonal antibodies).

The present invention also provides a cell or cell line expressing an antibody as described herein in recombinant form. Suitable host cells for expression of a recombinant antibody as described herein include prokaryotes, yeast, insect or higher eukaryotic cells under the control of appropriate promoters. Prokaryotes include gram-negative or gram-positive organisms, for example, E. coli or Bacilli. Higher eukaryotic cells include established cell lines of mammalian origin as described below. Cell-free translation systems can also be employed. Various mammalian or insect cell culture systems are used to express the recombinant antibody. Expression of in mammalian cells may be preferred because such proteins are generally correctly folded, appropriately modified and completely functional. Examples of suitable mammalian host cell lines include COS-7 (monkey kidney-derived), L-929 (murine fibroblast-derived), C127 (murine mammary tumour-derived), 3T3 (murine fibroblast-derived), CHO (Chinese hamster ovary derived), HeLa (human cervical cancer-derived) and BHK (hamster kidney fibroblast-derived) cell lines.

The present invention also provides an expression vector, capable of expressing an antibody as described herein. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are known to those of skill in the art.

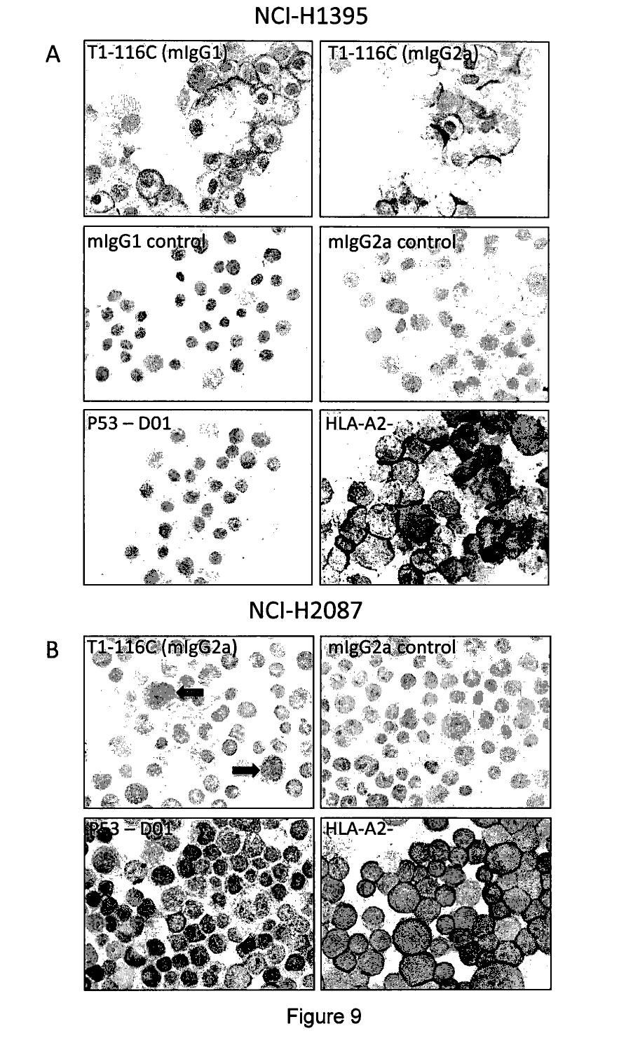

The present invention also provides the use of an antibody as defined herein in an in vitro method for determining the level of cellular antigen presentation of human p53₆₅₋₇₃ by HLA-A*0201. It has been realised by the present inventors that an antibody as defined herein may furthermore be useful as an analytical tool, for assessing in vitro the level of antigen presentation by a variety of cell types. The presence, absence or level of human p53₆₅₋₇₃ presentation by HLA-A*0201 may be determined by contacting one or more cells with a purified antibody as defined herein, and determining cell surface binding using standard analytical means known to the skilled person. For example, binding may be assessed qualitatively, semi-quantitatively or quantitatively; preferred techniques include fluorescence—activated cell sorting (FACS), immunohistochemistry, immunofluorescence and cell-based enzyme-linked immunosorbant assay (ELISA). Appropriate negative and positive controls will be apparent to the skilled person, depending on the technique used and cell type to be analysed. As an example only, to improve confidence an antibody specific for HLA-A2*0201 may be used (for example mAb BB7.2—see Example; FIG. 9) to confirm HLA-A2*0201 expression on test cell surfaces. The cell type to be analysed may include, but is not limited to, professional antigen-presenting cells (APCs); for example macrophages, B cells and dendritic cells. However, non-APCs of a variety of cell types can also acquire MHC I-peptide complexes for presentation to T cells, from neighbouring cells via intercellular contact (trogocytosis) and secreted membrane vesicles. The use of the antibody as defined herein for determining the level of cellular p53₆₅₋₇₃ antigen presentation by HLA-A*0201 in such cells is also within the purview of the invention. In addition, tumour cell or non-malignant cell presentation of p53₆₅₋₇₃ by HLA-A*0201 (when said cells have been obtained from a subject having a tumour/cancer) may be assessed, with implications for the therapy of the subject (see below). When tumour cell antigen presentation is assessed, a tumour-specific marker (which will vary according to cancer type) may be included to locate tumour cells within a heterogeneous cell population in a primary sample.

The present invention also provides an in vitro method for determining the suitability of a subject (preferably having the haplotype HLA-A*0201) having a tumour/cancer to undergo immunotherapy; comprising contacting one or more cells obtained from the subject with an antibody as defined herein, and determining the presence, absence or level of binding of said antibody to the surface of said one or more cells; wherein

(1) said one or more cells comprise tumour cells of the tumour/cancer; and wherein cell surface binding is a positive indication of the suitability of the subject to undergo said immunotherapy; or
(2) said one or more cells comprise non-malignant cells, and wherein cell surface binding is a negative indication of the suitability of the subject to undergo said immunotherapy;
wherein said immunotherapy is to be specific for human p53₆₅₋₇₃ presented by HLA-A*0201.

The presence, absence or level of binding of said antibody to the surface of said one or more cells may be determined using standard analytical means known to the skilled person. For example, binding may be assessed qualitatively, semi-quantitatively or quantitatively; preferred techniques include fluorescence—activated cell sorting (FACS), immunohistochemistry, immunofluorescence, cell-based enzyme-linked immunosorbant assay (ELISA). Positive and negative controls for cell surface binding will be apparent to the skilled person; for example, strongly positive p53+/HLA-A2+ cell lines, and negative cell lines or tumour cells of a patient subgroup with low endogenous tumour cell p53₆₅₋₇₃ presentation by HLA-A2*0201, respectively. A clinically relevant cut-off for therapy may be determined by the skilled person by a variety of means; following methods available in the art. Positive and negative controls may be used to set upper and lower thresholds respectively, and the distribution of patient data viewed relative to said thresholds. By way of example, clinical response may be correlated to mean fluorescence intensity when using FACS, number of bound antibodies per cell when using FACS in combination with PE-conjugated antibody and QuantiBRITE® PE beads (see Example; Quantitation of antibody molecules bound per target cell), or staining signal or frequency of positivity when using immunohistochemistry.

According to this aspect of the invention, if (1) applies, the subject preferably has a tumour/cancer selected from the group consisting of lung cancer, melanoma, osteosarcoma, colon cancer, breast cancer, chronic lymphocytic leukaemia, follicular lymphoma, mast cell leukaemia, diffuse large B-cell lymphoma, prostate cancer, pancreatic cancer, ovarian cancer and mantle cell lymphoma; more preferably said group consists of lung cancer, melanoma, osteosarcoma, colon cancer, breast cancer, chronic lymphocytic leukaemia, follicular lymphoma, mast cell leukaemia, diffuse large B-cell lymphoma, pancreatic cancer, and mantle cell lymphoma. Binding of an antibody as defined herein to said tumour cell types is indicative of a positive clinical response to immunotherapy as defined above.

If (2) applies, said one or more cells preferably comprise peripheral blood mononuclear cells. Binding of an antibody as defined herein to such non-malignant cells is indicative of off-target reactivity during immunotherapy (specific to the subject). Therefore, immunotherapy as defined above would be disfavoured in the subject.

The immunotherapy is preferably selected from vaccination, administration of an antibody or pharmaceutical composition as defined herein, and TCR-based immunotherapy.

It is to be noted that the diagnostic test defined herein may be used by the skilled person in conjunction with the PET and MRI in vivo use embodiments (as defined above), to improve response and/or safety predictions concerning the subject intended to undergo said immunotherapy.

Cell line deposits prefixed by “ATCC” refer to deposits made at American Type Culture Collection (ATCC), 10801 University Boulevard, Manassas, Va. 20110-2209; cell line deposits prefixed by “DSMZ” refer to deposits made at Leibniz-lnstitut DSMZ—Deutsche Sammlung von Mikroorganismen and Zellkulturen GmbH, Inhoffenstr. 7B 38124 Braunschweig. The prefix “PMID” refers to a PubMed manuscript ID (http://www.ncbi.nlm.nih.gov/pubmed, as accessed 28 August 2015).

The invention is now illustrated by reference to the following non-limiting Example.

### Example

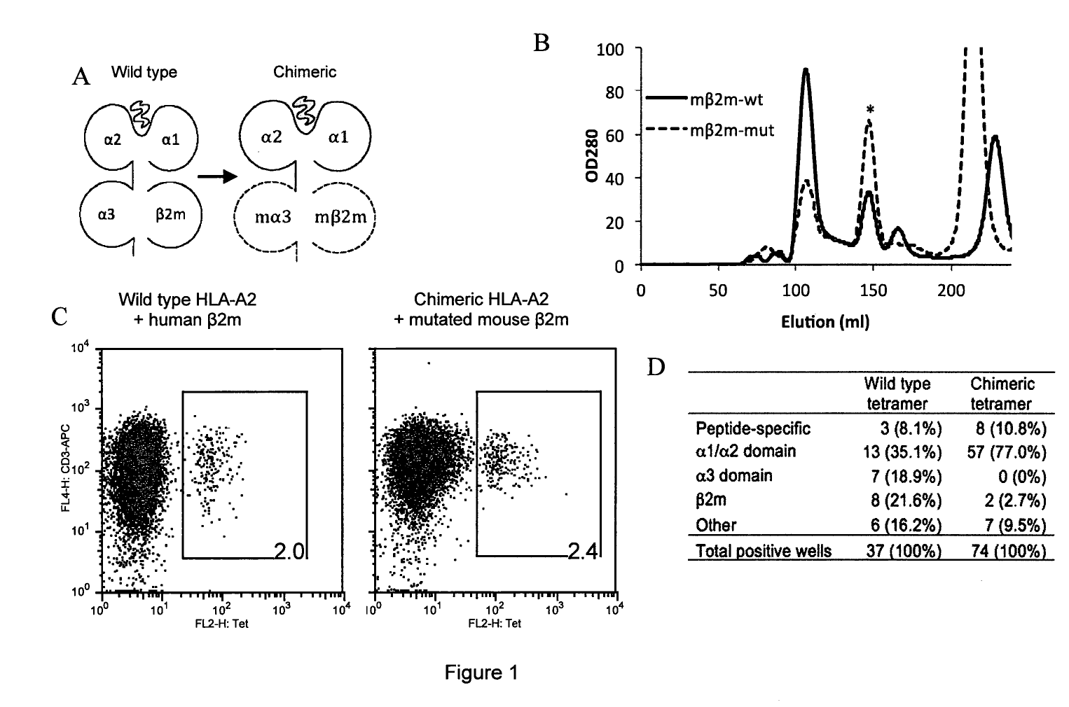

The present inventors used human tetramers composed of the human MHC class I protein HLA-A*0201, β2-microglobulin (β2m) and an immunogenic peptide derived from the human tumour suppressor protein p53 as an immunogen with which to generate monoclonal antibodies. As a large number of antibodies recognised human components of the tetramer that did not comprise the desired epitope (e.g. the undesirable human α3 domain and β2m) chimeric tetramers were also engineered to replace these domains with the murine BALB/c counterparts to reduce undesirable immunogenicity (FIG. 1). This included mutations of Pro53Ser, His54Asp, Met71His, Met74Leu in the murine β2m protein to increase monomer re-folding efficiency. These chimeric tetramers were also used as immunogens for antibody production.

The human p53 peptides selected were those with proven endogenous presentation[54-56], that had been tested in clinical trials of p53 vaccines.[28] Wild type human p53 peptides, located N-terminal to the most common mutations leading to premature termination of translation (R196X and R213X), were chosen to advantageously enable therapeutic targeting of the widest range of HLA-A2⁺ patients with p53 epitope presentation on their tumours (Table 1).

TABLE 1
Wild type p53 peptides selected for TCRm antibody production

| Peptide | Peptide1 | Peptide 2 |
|---|---|---|
| amino acid coordinates | 65-73 | 187-197 |
| human sequence | RMPEAAPPV | GLAPPQHLIRV |
| mouse sequence | RVSGAPAAQ | GLAPPQHLIRV |
| human/mouse homology | 22% | 100% |

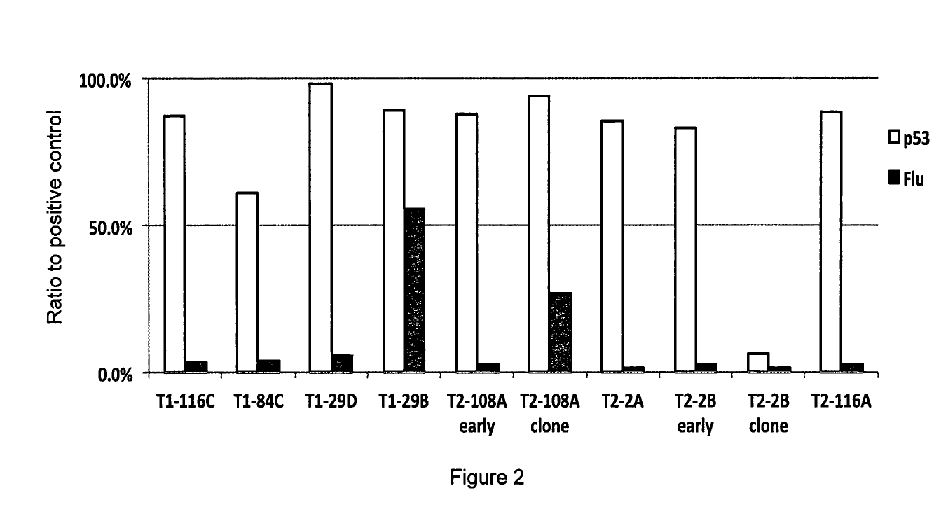

Monoclonal antibodies were generated against human or chimeric (human/murine) tetramers containing either the wild type p53₆₅₋₇₃ or p53₁₈₇₋₁₉₇ peptide by immunising MF1 or BALB/c mice (T1-116C was generated from MF1 mice). Splenic B cells taken from the immunised mice were then fused with a myeloma cell line (NS0) to create immortalised antibody secreting hybridomas. Hybridoma supernatants from individual hybridoma colonies were tested, by ELISA assay, for recognition of a human tetramer presenting the immunising peptide, irrespective of whether the immunogen was a human or chimeric tetramer (FIG. 2). A human tetramer containing a non-target peptide (derived from Influenza A virus, flu peptide GILGFVFTL) was used to detect antibodies non-specifically recognising the MHC complex/tetramer backbone. This first round of screening identified multiple potential p53 TCRm antibodies that exhibited preferential and/or specific binding to a tetramer containing the immunising peptide. The T1-29B antibody only exhibited increased binding to its p53₆₅₋₇₃ target containing tetramer and was excluded as having insufficient specificity. The T2-108A antibody initially exhibited the desired specificity for its p53₁₈₇₋₁₉₇ target, but after cell line cloning showed an undesirable increase in binding to the control tetramer. This could potentially be a consequence of higher antibody concentrations being tested. Antibody production by hybridoma cell lines is inherently unstable and the T2-2B antibody clones subsequently failed to show sufficiently effective binding to their p53₁₈₇₋₁₉₇ target. Four TCRm cell lines (T1-116C, T1-29D, T2-108A and T2-2A) were cloned by single cell dilution to generate cell lines secreting a single monoclonal antibody. T1-84C, could not be cloned or purified (despite repeated attempts) and thus only supernatant was evaluated and T2-116A was not cloned as pilot experiments showed little cell line binding.

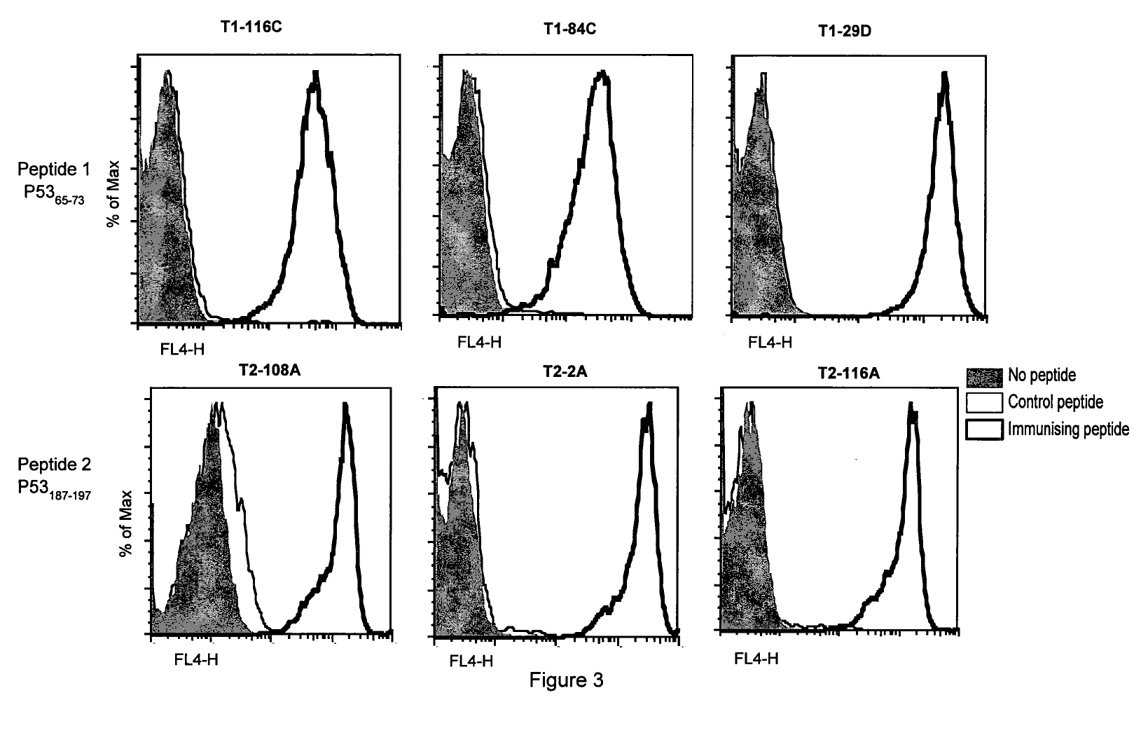

The p53 TCRm antibodies were tested for their ability to recognise their target peptide presented on the cell surface of T2 cells in the context of HLA-A2. T2 is a hybrid cell line that is only able to naturally express low amounts of the HLA-A2 protein on the cell surface, because of its deficiency in the transporter associated with antigen processing (TAP).[57] This deficiency in peptide transport can be overcome by pulsing the T2 cells with MHC class I binding peptides that then stabilise the HLA-A2/peptide complex on the cell surface. T2 cells were pulsed with either the target p53 peptide or the control flu-derived peptide. By virtue of exhibiting positive binding for T2 cells pulsed with their p53 target peptide and not binding T2 cells pulsed with an irrelevant control flu peptide, five antibodies were considered to exhibit specificity for recognising T2 cells presenting p53 epitopes (FIG. 3, Table 2). Subsequent data identified even higher undesirable cross-reactivity with the control flu peptide for later batches of the T2-108A antibody in both the ELISA (FIG. 2) and T2 assay (data not shown). This antibody only preferentially bound T2 cells pulsed with the target p53 peptide and thus was not considered to be sufficiently specific for further therapeutic development. Antibodies T1-116C, T1-29D, T1-84C, T2-2A and T2-116A were confirmed to exhibit specific recognition of their target p53 peptide in the T2 presentation assay. In contrast, one anti-p53 TCR promiscuously capable of recognising both the p53₆₅₋₇₃ and p53₁₈₇₋₁₉₇ peptides has been reported in the literature.[56]

TABLE 2
TCRm antibodies preferentially recognising p53
peptide containing tetramers in a T2 assay.

| Hybridoma | P53 peptide | Tetramer | Isotype | T2 specificity |
|---|---|---|---|---|
| T1-116C | P53₆₅₋₇₃ | human | IgG1 | specific |
| T1-84C | P53₆₅₋₇₃ | chimeric | IgG2b | specific |
| T1-29D | P53₆₅₋₇₃ | chimeric | IgG1 | specific |
| T2-2A | P53₁₈₇₋₁₉₇ | chimeric | IgG2a | specific |
| T2-116A | P53₁₈₇₋₁₉₇ | chimeric | IgG1 | specific |
| T2-108A | P53₁₈₇₋₁₉₇ | human | IgG1 | NS |

NS indicates insufficient specificity.

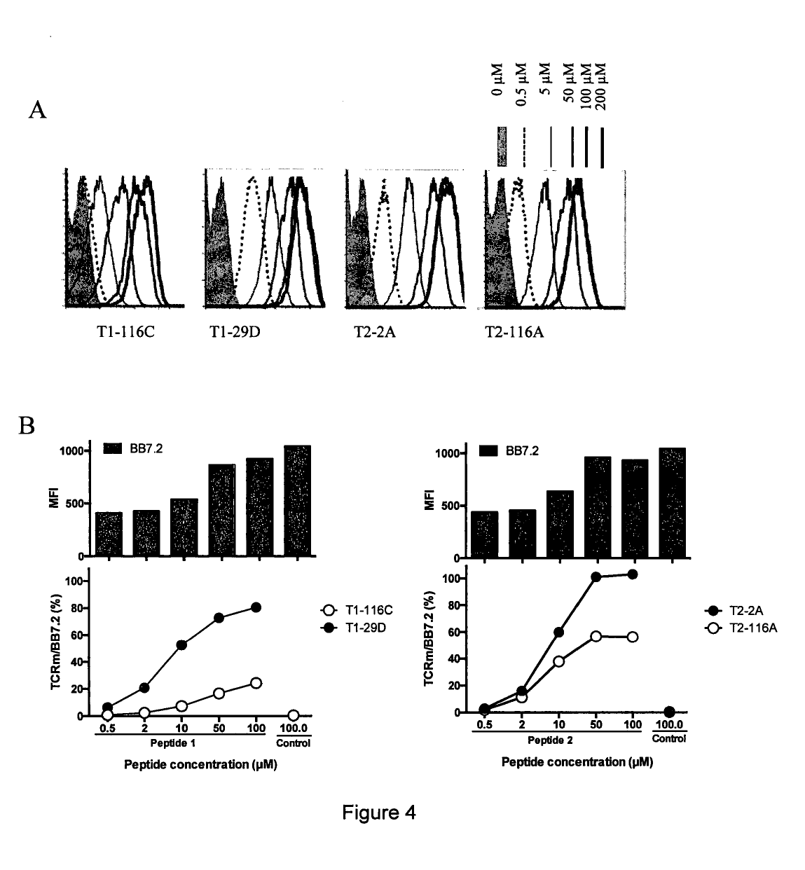

T2 cells were pulsed with decreasing concentrations of peptide to compare the relative abilities of the four specific TCRm antibodies (for which we had purified antibody) to detect their target peptide. Staining with BB7.2 (anti-HLA-A2) was performed to determine the levels of MHC class I presentation. HLA-A2 levels at the cell surface decreased at lower peptide concentrations, which were thus insufficient to maximise MHC class I presentation. All four antibodies effectively labelled T2 cells pulsed with 50 μM-200 μM of their target peptide and started to exhibit reduced binding at 5 μM peptide (FIG. 4). Only T1-116C was unable to markedly detect surface presentation of its target peptide on T2 cells pulsed with a 500 nM peptide concentration.

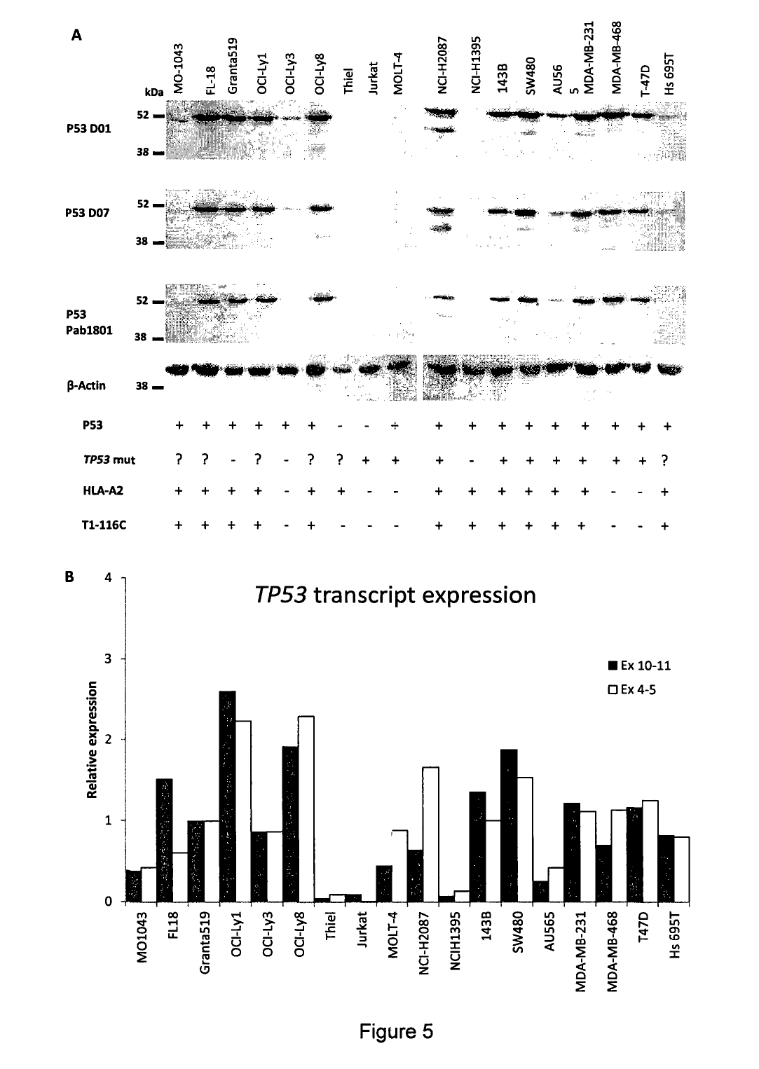

The ability of the p53 TCRm antibodies to detect the endogenously presented p53 peptide on cancer cell lines was investigated by flow cytometry. The HLA-A2 status of each cell line was determined by flow cytometry using antibody BB7.2 (data not shown) and their p53 status was determined by RT-qPCR to detect transcript expression and Western blotting using three commercial anti-p53 antibodies against the p53 N-terminus (FIG. 5).

Only the antibody of the present invention, T1-116C, was able to widely label (n=15/38, 39.5%) the surface of a range of cancer cell lines derived from different tumour types (FIG. 6, Table 3). The T1-116C antibody immunolabelling was almost exclusively restricted to HLA-A2+ cancer cell lines (the exception being HL-60 which was still bound by T1-116C despite being HLA-A2 negative). Staining 15/22 (68.2%) of the HLA-A2+ cell lines, 15/21 (71.4%) of the HLA-A2+ cell lines with confirmed p53 protein expression and only one of the 17 HLA-A2-negative cell lines, of which 11 expressed detectable p53 protein (FIGS. 5, 6 and Table 3). There was also no T1-116C labelling of the HLA-A2+ Thiel cell line in which p53 protein expression was undetectable by either Western blotting or immunocytochemistry (FIGS. 5, 6 and Table 3). However, HL-60 cells lacked p53 protein expression and were bound by the T1-116C antibody. The epitope bound by T1-116C on HL60 cells is unknown, but the binding does not seem to represent epitope independent binding by the Fc receptors expressed on HL-60 cells, as control antibodies with the same isotype did not bind. The level of p53 protein expression was not an accurate indicator of the intensity of T1-116C staining. This is consistent with reports of p53 turnover, rather than steady-state levels, determining the presentation of epitopes by MHC class I to CTLs.[20] T1-116C was able to recognise cell lines with either wild type p53 or a variety of different TP53 mutations. Interestingly, three (MDA-MB-435, MCF-7 and KMH2) of the six HLA-A2⁺/p53⁺ cell lines that were not stained by T1-116C had been reported in the IARC database as having wild type TP53 and only expressed low levels of the protein. The T1-116C antibody was able to label cell lines derived from a variety of different cancer subtypes including, lung cancer, osteosarcoma, colon cancer, breast cancer, melanoma, pancreatic cancer and haematological malignancies including chronic lymphocytic leukaemia, follicular lymphoma, mantle cell lymphoma and diffuse large B-cell lymphoma.

A minority of cancer cell lines were also labelled by T2-108A or T2-116A. T2-108A exhibited weak labelling of NCI-H1395 and MO-1043, T2-116A weakly labelled NCI-H1395 and SW480 (FIG. 6). These antibodies were not developed further as they failed to effectively bind other HLA-A2+/p53+ cancer cell lines that were labelled by T1-116C. There is currently no obvious explanation for why T1-116C was preferentially able to bind cancer cells, while other TCRm antibodies, that showed stronger labelling of T2 cells pulsed with their target p53 peptide did not. Thus only one of five p53 TCRm antibodies, capable of specifically recognising the target p53 peptide presented by HLA-A2, was considered to have sufficiently widespread reactivity with the endogenously presented peptide on HLA-A2⁺/p53⁺ cancer cells to pursue it further as a potential therapeutic.

TABLE 3
T1-116C immunoreactivity with cancer cell lines, their HLA-A2
status, TP53 mutation status and p53 protein expression.

| | | HLA-A2 | | p53 | |
|---|---|---|---|---|---|
| Cell line | Tumour | status | p53 MUT | protein | T1-116C |
| NCI-H2087 | Lung | + | V157F | ++ | + |
| NCI-H1395 | Lung | + | WT | +/− | +++ |
| CALU6 | Lung | − | R196stop | − | − |
| COR-L23 | Lung | − | WT | − | − |
| NCI-H1299 | Lung | − | WT/NULL | − | − |
| NCI-H1975 | Lung | − | R273H | +++ | − |
| NCI-H1930 | Lung | + | G245R | ++ | − |
| A2058 | Melanoma | − | V274F | +/− | − |
| G361 | Melanoma | − | | +/− | − |
| HS-695T | Melanoma | + | | +/− | ++ |
| 143B | Osteosarcoma | + | R156P | +++ | + |
| SW480 | Colon | + | P309S | +++ | ++ |
| | | | R273H | | |
| Colo-205 | Colon | + | G266E | ++ | + |
| | | | Y103_L111 > L | | |
| Colo-678 | Colon | + | WT | ++ | + |
| AU565 | Breast | + | R175H | + | + |
| MDA-MB-231 | Breast | + | R280K | +++ | ++ |
| MDA-MB-453 | Breast | + | WT/MUT | +/− | − |
| MDA-MB-468 | Breast | − | R273H | +++ | − |
| MCF-7 | Breast | + | WT | +/− | − |
| T47D | Breast | − | L194F | ++ | − |
| PANC-1 | Pancreas | + | V272A | ++ | + |
| | | | R273H | | |
| MO-1043 | CLL | + | | +/− | ++ |
| FL-18 | FL | + | | +++ | + |
| Granta-519 | MCL | + | WT | +++ | +++ |
| OCI-Ly1 | DLBCL | + | | +++ | ++ |
| OCI-Ly8 | DLBCL | + | | +++ | +++ |
| SUDHL6 | DLBCL | + | | ++ | − |
| OCI-Ly3 | DLBCL | − | WT | +/− | − |
| KMH2 | cHL | + | WT | +/− | − |
| THIEL | Myeloma | + | | − | − |
| Daudi | BL | − | G266Q | + | − |
| | | | R213stop | | |
| CCRF-CEM | T-ALL | − | R175H | +++ | − |
| | | | R248Q | | |
| MOLT-4 | T-ALL | − | R248Q | +/− | − |
| | | | R306stop | | |
| Jurkat | T-ALL | − | R196stop | − | − |
| | | | T256A | | |
| | | | D259G | | |
| | | | S260A | | |
| RPMI 8402 | T-ALL | − | R273C | ++ | − |
| HUT78 | CTCL | − | R196stop | + | − |
| KARPAS-299 | ALCL | − | R273C | ++ | − |
| SUDHL-1 | ALCL | + | | ++ | − |
| HL60 | APL | − | NULL | − | + |

The intensity of immunolabelling is indicated as very weak (+/−), weak (+), moderate (++) or strong (+++). Chronic lymphocytic leukaemia (CLL), follicular lymphoma (FL), mantle cell lymphoma (MCL), diffuse large B-cell lymphoma (DLBCL), classical Hodgkin lymphoma (cHL), Burkitt lymphoma (BL), T-cell acute lymphoblastic leukaemia (T-ALL), cutaneous T-cell lymphoma (CTCL), ALK⁺ anaplastic large cell lymphoma (ALCL), acute promyelocytic leukaemia (APL). NT are samples where p53 protein status has not yet been tested. Mutations in p53 (MUT) are indicated with the original amino acid, codon position, and alteration; data were retrieved from the IARC TP53 database (http://p53.iarc.fr/CellLines.aspx). WT refers to wild type TP53, WT/NULL and WT/MUT indicates either null or mutated TP53 reported as well as wild type in IARC TP53 database.

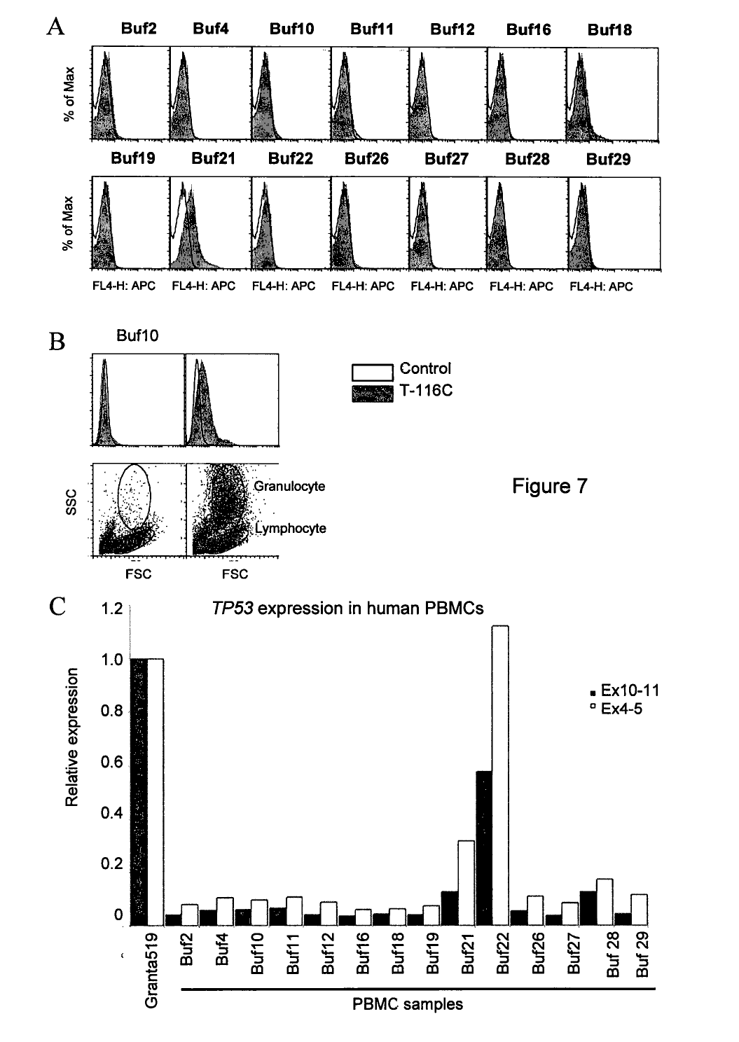

The expression of p53 in normal tissues has been linked to their radiation-sensitivity, with hematopoietic tissues being among the normal adult tissues exhibiting the highest levels of p53 protein expression.[58, 59] Normal circulating peripheral blood mononuclear cells (PBMCs) have been demonstrated to express the p53 protein, and levels can be increased even further by exposure to gamma irradiation.[60] Flow cytometry analysis was performed to investigate whether normal PMBCs presented sufficient copies of the wild type p53₆₅₋₇₃ peptide to enable binding of the T1-116C antibody. 13/14 (92.9%) PBMC preparations from HLA-A2⁺ donors were negative for T1-116C staining when tested with the hybridoma supernatant (FIG. 7A) or the purified antibody (FIG. 7B). The single positive patient (Buf21), who only exhibited weak staining, had an abnormally high expansion of granulocytes, which may be indicative of some potential abnormality. A non-exhaustive list of potential health problems associated with such granulocytosis includes leukaemia, bacterial infection and autoimmune disorders. Two patients (Buf21 and Buf22) had elevated TP53 transcript expression (FIG. 7C), although only in Buf21 was this accompanied by T1-116C binding. These data indicate that the T1-116C antibody is able to discriminate between p53⁺/HLA-A2⁺ normal and tumour cells. This is consistent with reports from studies using T cells that indicated malignant cells have increased p53 epitope presentation.[19-21] Testing normal PBMCs from patients for T1-116C binding prior to therapy would enable individuals presenting abnormally high levels of p53 in their normal tissues to be excluded from receiving this p53-targeted therapy.

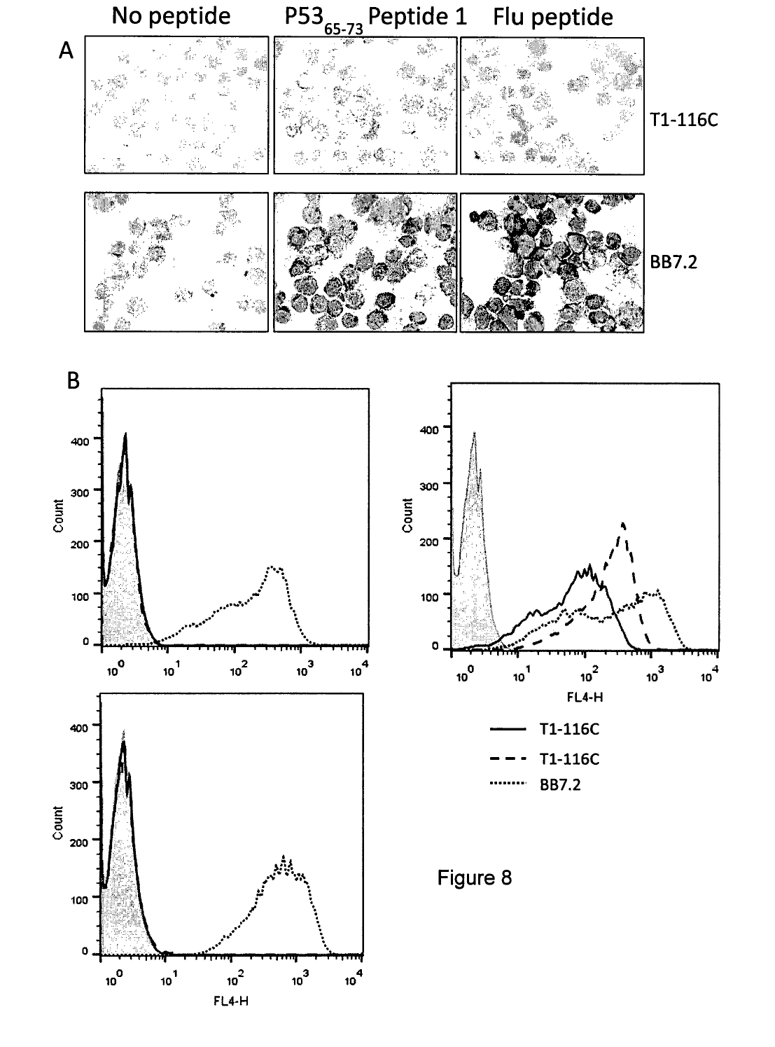

The ability of the T1-116C to stain tumour cell lines by immunocytochemistry (ICC) was investigated, as this experimental technique is particularly amenable to routine clinical screening of antibody reactivity with normal and malignant solid human tissues. The T1-116C antibody was able to recognise its epitope on acetone-fixed T2 cells pulsed with the target p53₆₅₋₇₃ peptide. T1-116C staining was not observed when T2 cells were pulsed with the irrelevant flu-derived peptide indicating that ICC specifically demonstrated recognition of the presented p53 peptide (FIG. 8). T1-116C labelling of NCI-H1395 cells, which were strongly T1-116C stained by flow cytometry, was also detectable by ICC using both an IgG1 and IgG2a format of the T1-116C antibody (FIG. 9A). No NCI-H1395 staining was detected using equivalent concentrations of isotype matched control antibodies.

Future studies will address whether ICC replicates the pattern of T1-116C staining detected by flow cytometry and whether it is sufficiently sensitive to detect cell lines that only exhibit weak positivity by flow cytometry. Preliminary data from ICC on NCI-H2087 lung cancer cells (which are only weakly T1-116C labelled by flow cytometry) suggests it is possible to detect T1-116C binding, although only in a minority of cells (FIG. 9B).

An in silico approach to predicting potential TCR cross reactivity was developed following the death of the first two patients treated with affinity-enhanced MAGE-A3 TCR targeted T cells that recognised the EVDPIGHLY peptide in the context of HLA-A*01.[61, 62] Despite a lack of MAGE-A3 expression in the heart, autopsy findings reported severe myocardial damage in both patients. An amino acid scanning approach enabled identification of a TCR binding peptide from the striated muscle-specific protein Titin (ESDPIVAQY) as the cross-reactivity responsible for the previously unpredicted cardiovascular toxicity.[61, 62] Importantly the use of a murine model system would not have predicted this toxicity as the murine Titin peptide was not recognised by the MAGE-A3 TCR.[61] Thus highlighting the importance of additional strategies for predicting off-target toxicity.

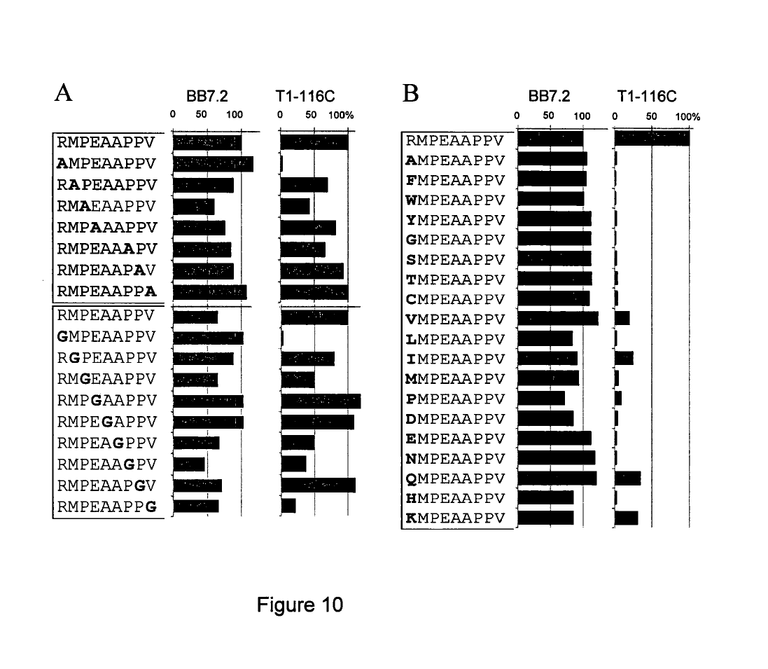

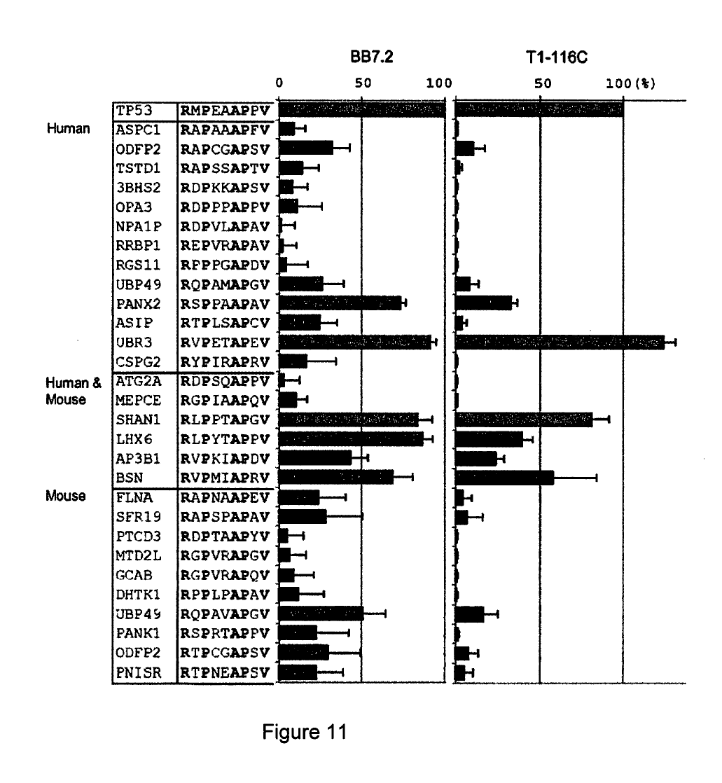

The approach of alanine and glycine replacement of individual amino acids in the target peptide was adopted for identifying the residues in the p53₆₅₋₇₃ peptide that were important for T1-116C antibody recognition. T2 cells pulsed with the wild type or mutated peptides were tested (n=3) for the ability of the peptides to bind HLA-A2 (BB7.2 staining) and for binding of T1-116C (FIG. 10A). When either alanine or glycine substitution resulted in a large decrease in T1-116C binding (>50%) then the residue at this position was considered to contribute to antibody binding and/or peptide presentation. This approach derived a consensus of RXPXXAPXV, where X is an amino acid that can be substituted into the RAPAAAPFV p53₆₅₋₇₃ peptide without reducing T1-116C antibody binding by >50%. The first arginine (R) amino acid was absolutely required for T1-116C binding. There was no reduction in the ability of this mutated peptide to bind HLA-A2, indicating that the first arginine is an essential component of the T1-116C epitope. No other amino acid could be successfully substituted into this essential position to restore >50% T1-116C binding (FIG. 10B). A directed search of the UniProtKB/Swiss-Prot protein database with the RXPXXAPXV consensus sequence, using the ScanProsite tool, identified 20 human proteins (including p53) that could generate potentially T1-116C binding peptides, in terms of their sequence identity (Table 4). The same directed search of the murine protein database was performed to identify peptides that could be presented in HLA-A2 transgenic mice. As illustrated in FIG. 11, 6/20 peptides were identical in the mouse, while an additional 10 peptides from mouse proteins also contained the T1-116C consensus.

The ability of each human peptide to bind HLA-A*0201 was then predicted in silico using the BIMAS http://www-bimas.cit.nih.gov/molbio/hla_bind/) and SYFPETHI (http://www.syfpeithi.de) algorithms (Table 4). The three non-target (non-p53) peptides with high HLA-A*0201 binding score predictions from both algorithms, and p53, are SHANK1, LHX6 and UBR3.

All of the potentially cross-reactive peptides were synthesised and analysed (n=3) for HLA-A2 (BB7.2 antibody) and T1-116C binding in T2 assays (FIG. 11). The BIMAS and SYFPETHI algorithms effectively predicted the three strong HLA-A2 binding peptides but failed to accurately identify which among the lower scoring peptides could also exhibit HLA-A2 binding. Peptides, derived from three antigens, SHANK1, UBR3 and BSN were able to achieve >50% of the level of T1-116C binding observed with the target p53₆₅₋₇₃ peptide, although the BSN peptide was only stained strongly in one of three replicate experiments. These peptides share 5/9 amino acid identity with the p53 peptide e.g. all the non-essential amino acids are changed in each peptide. No other peptides demonstrated >50% of the T1-116C labelling observed with its target p53₆₅₋₇₃ peptide. These data indicate that this in silico strategy, which has not previously been applied to characterising the specificity of a TCRm antibody, was indeed able to identify HLA-A2 binding peptides that have the potential to be recognised by the T1-116C TCRm antibody. The data also validate the stringency threshold of >50% reduction in T1-116C binding that was selected to predict residues required for T1-116C binding. A modest decrease of <50%, observed for substitutions of the methionine at position 2 (P2), was considered insufficient to be necessary for T1-116C binding, despite the P2 position having a widely established role in the literature in stabilising HLA-A2 peptide binding. HLA-A2 presented peptides with an alteration in the P2 position were recognised by the T1-116C antibody and indeed none of the 19 potentially cross-reactive peptides would have been identified if there had been a consensus sequence requirement for a methionine at position P2.

TABLE 4
Human proteins containing peptides derived from
alanine and glycine replacement of individual
amino acids containing the T1-116C consensus of
RXPXXAPXV, their in silico predicted HLA-A*0201
binding scores and experimentally determined
HLA-A2 binding (BB7.2 staining) and T1-116C
binding.

| | | | | | T1- |
|---|---|---|---|---|---|
| Protein | Peptide | BIMAS | SYFPETHI | BB7.2 | 116C |
| P53 | RMPEAAPPV | 205.951 | 21 | +++ | +++ |
| ASPC1 | RAPAAAPFV | 5.313 | 15 | +/− | − |
| ODFP2 | RAPCGAPSV | 0.966 | 19 | + | +/− |
| TSTD1 | RAPSSAPTV | 0.966 | 17 | +/− | − |
| 3BHS2 | RDPKKAPSV | 0.097 | 14 | +/− | − |
| OPA3 | RDPPPAPPV | 0.097 | 14 | + | − |
| ATG2A | RDPSQAPPV | 0.097 | 11 | +/− | − |
| NPA1P | RDPVLAPAV | 0.097 | 13 | +/− | − |
| RRBP1 | REPVRAPAV | 1.352 | 12 | +/− | − |
| MEPCE | RGPIAAPQV | 0.454 | 11 | +/− | − |
| SHANK1 | RLPPTAPGV | 69.552 | 24 | +++ | +++ |
| LHX6 | RLPYTAPPV | 69.552 | 20 | +++ | + |
| RGS11 | RPPPGAPDV | 0.454 | 15 | +/− | − |
| UBP49 | RQPAMAPGV | 7.052 | 12 | + | +/− |
| PANX2 | RSPPAAPAV | 0.454 | 14 | +++ | + |
| ASIP | RTPLSAPCV | 0.966 | 15 | + | − |
| UBR3 | RVPETAPEV | 24.952 | 19 | +++ | +++ |
| AP3B1 | RVPKIAPDV | 6.086 | 15 | ++ | + |
| BSN | RVPMIAPRV | 6.086 | 15 | +++ | +++ |
| CSPG2 | RYPIRAPRV | 0.01 | 11 | + | − |

The intensity of antibody staining is indicated as negative (−) 0-10% observed with the p53 peptide, very weak (+1-) >10%-<25% observed with the p53 peptide, weak (+) >25-<50% observed with the p53 peptide, moderate (++) >50-<75% observed with the p53 peptide or strong (+++) >75% observed with the p53 peptide. The strongest staining observed in any one of the three replicate experiments is indicated. Bold font indicates those peptides with the most effective T1-116C binding (>50% binding of that to p53).

In silico sequence analysis to predict potential epitope cross reactivity has identified several proteins (most notably SHANK1, UBR3, and BSN) with the potential to generate HLA-A2-bound peptides that could effectively bind the T1-116C antibody and thus give rise to off-target reactivity, weak binding was also observed to LHX6, PANX2 and AP3B1. Four of these peptides, Shank1, Lhx6, Ap3b1 and Bsn, were identical in the mouse, while the PANX2 and UBR3 peptides were not conserved in the orthologous protein. Importantly none of the peptides identified only in murine proteins showed strong T1-116C binding. Thus studies in HLA-A2 transgenic mice could be used for safety testing the T1-116C mAb in a system containing several of the non-p53 potential targets without any murine specific off-target binding. However, these data do not address whether these epitopes are indeed naturally processed and presented or the expression pattern of the target antigen.

Searches for known T-cell epitopes (with antigens exhibiting >25% T1-116C binding) in the Immune Epitope Database and Analysis Resource (http://www.iedb.org) identified the known p53 epitope but found no experimentally determined epitopes for SHANK1, LHX6, PANX2, UBR3 or BSN. MHC class I epitopes for AP3B1 had been identified, but these were not the same peptide or HLA haplotype. Web searching with T cell epitope and the antigen name identified a murine MHC class I epitope from Ubr3 and an MHC class II presented murine peptide from Bsn that did not overlap with peptides identified in the current study. Web searching with the peptide sequence, among the top hits on Google, also failed to identify T-cell epitopes for any of the antigens but did identify the p53 epitopes. Thus to the best of the inventors knowledge there is no existing evidence that the T1-116C cross-reactive peptides are naturally processed and presented within the context of human HLA-A*0201. This can be further investigated by overexpressing potentially cross reactive antigens in HLA-A2⁺/p53-negative cell lines and studying correlation between antigen expression levels in cell lines and T1-116C binding.

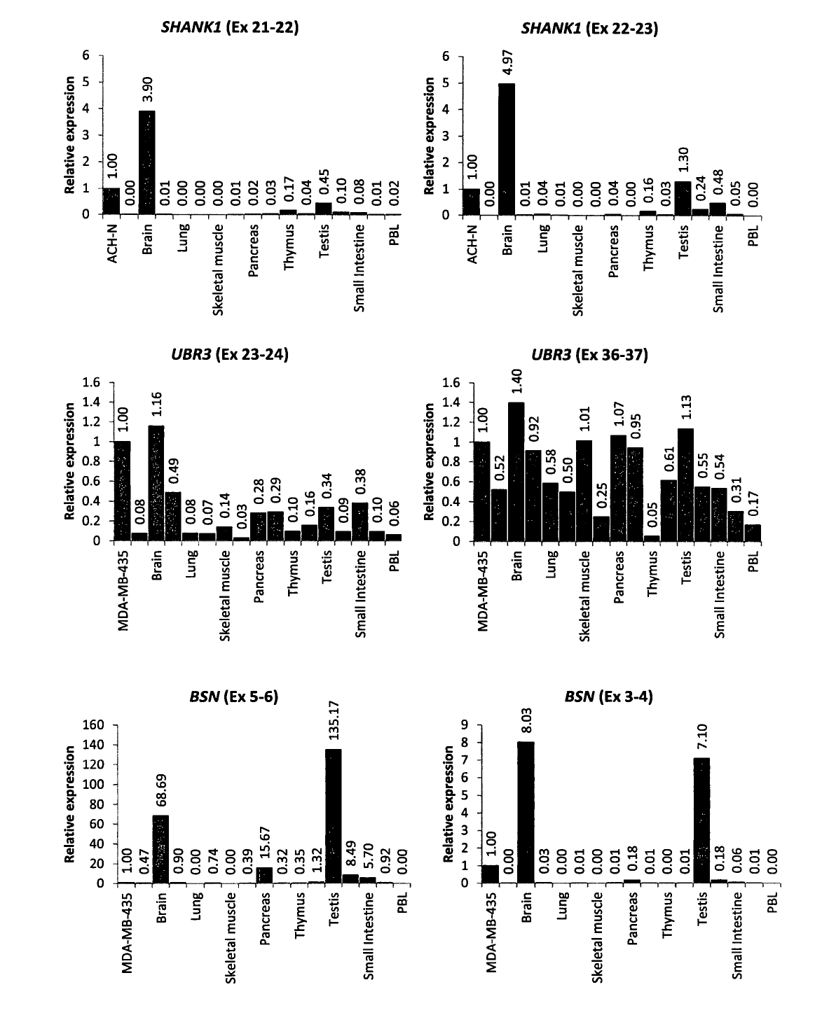

Normal human tissue expression patterns of these antigens were investigated to assess the potential risks of toxicity if there was off-target T1-116C binding. Although it should be noted that transcript expression does not guarantee protein expression or peptide presentation. The expression of SHANK1 and BSN transcripts was restricted in normal tissues with the brain and testis exhibiting the highest levels of normal tissue expression (FIG. 12). These tissues are immune privileged sites where the introduction of an antigen does not elicit an inflammatory immune response. The blood brain barrier itself is known to block access for therapeutic antibodies into the central nervous system, while Sertoli cells isolate germ cells in the testis from the blood. The expression of UBR3 was widespread in human tissues. Peripheral blood lymphocytes lacked T1-116C labelling, but were at the lower end of normal tissue UBR3 transcript expression.

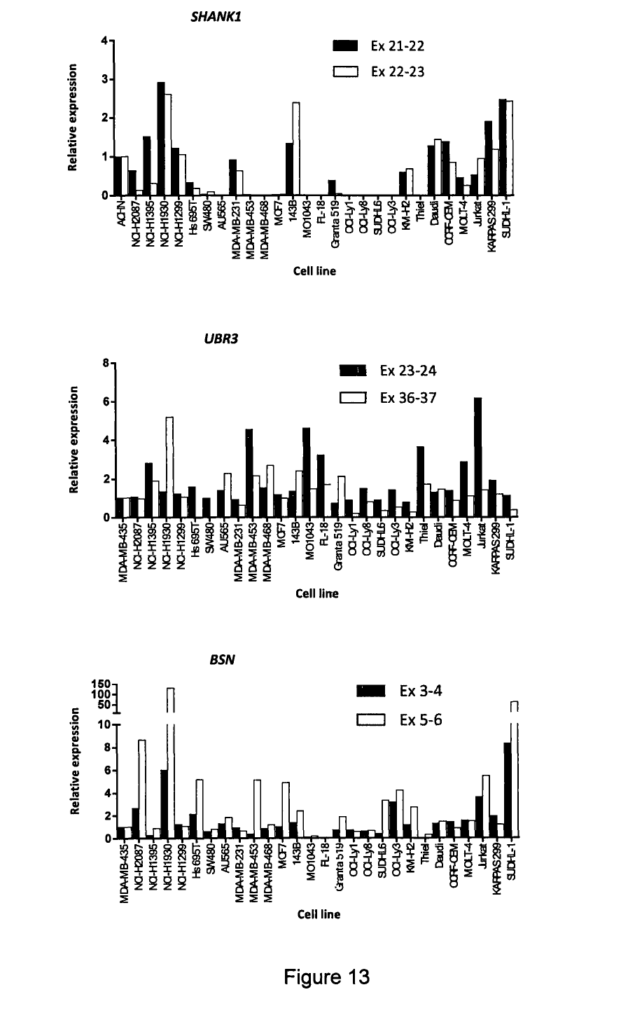

In cancer cell lines UBR3 transcript expression was frequently higher than the levels observed in normal tissues. UBR3 was particularly highly transcribed (FIG. 13) in the HLA-A2⁺ Thiel (p53-negative) and MDA-MB-453 (weakly p53⁺) cell lines, which both lacked T1-116C binding. If the UBR3 protein is also abundantly expressed in these cell lines, and the finding extends to a wider panel of cell lines, then this would be highly suggestive of the cross-reactive peptide not being presented. UBR3 was also expressed in HLA-A2⁺ NCI-H1930 cells, which lacked T1-116C binding. SHANK1 and BSN transcripts were most abundantly expressed in NCI-H1930 and SUDHL1 cells, both cell lines were HLA-A2+ but were not labelled by T1-116C. Further studies of potentially cross-reactive protein expression and presentation will clarify whether T1-116C can bind naturally processed and presented epitopes from proteins other than p53.

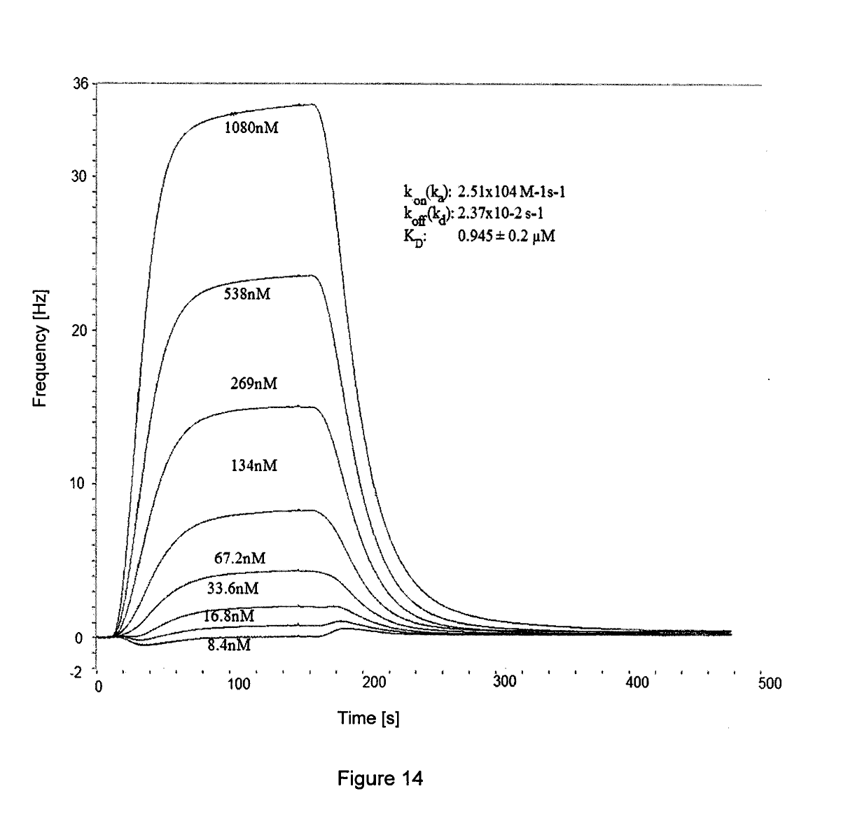

A human IgG1 chimeric T1-116C antibody was transiently expressed in CHOK1SV GS-KO cells using the GS Xceed™ vector system by Lonza Biologics plc. The recombinant antibody exhibited a reasonable yield (9.3 mg from 400 ml culture) with only a small percentage (˜1.6%) of higher molecular weight impurities. The affinity of this human IgG1 chimeric T1-116C antibody for a human HLA-A2 tetramer containing the p53₆₅₋₇₃ peptide was 0.977 μM using a Quartz crystal Microbalance (QCM) assay performed by Lonza Biologics plc (FIG. 14).

The number of available epitopes present on the cell surface for antibody binding is an important determinant of therapeutic antibody activity. A standard curve of PE-coupled calibration beads (QuantiBRITE PE beads) was used to estimate the number of PE-conjugated T1-116C antibodies bound to the surface of peptide pulsed T2 cells and cancer cell lines. T2 cells were pulsed with increasing concentrations of the p53₆₅₋₇₃ peptide. Approximately 100 bound T1-116C molecules per cell were detectable above background levels in this assay. This is comparable to p53₂₆₄₋₂₇₂/HLA-A2 TCR binding (200-300 binding sites per cell) detected using a soluble TCR with the same assay system.[50] Approximately 2000 T1-116C antibody molecules were bound per cell for MDA-MB-231, while approximately 5800 were bound per cell for OCI-Ly8.

TABLE 5
Quantitation of T1-116C binding sites per target cell.

| Cell line | Peptide (T2 only) | T1-116C-PE per cell |
|---|---|---|
| T2 | Flu-100 μM | 179 |
| | p53₆₅₋₇₃-100 μM | 60955 |
| | p53₆₅₋₇₃-50 μM | 21954 |
| | p53₆₅₋₇₃-10 μM | 2190 |
| | p53₆₅₋₇₃-2 μM | 440 |
| | p53₆₅₋₇₃-0.5 μM | 119 |

| | |
|---|---|
| FL-18 | 1329 |
| Granta-519 | 7491 |
| MO1043 | 4145 |
| OCI-Ly1 | 4016 |
| OCI-Ly8 | 5819 |
| AU565 | 1617 |
| Hs695T | 1101 |
| MDA-MB-231 | 1956 |
| NCI-H1395 | 15291 |
| NCI-H2087 | 3975 |
| SW480 | 551 |
| 143B | 1019 |

TCRm antibodies can be used to deliver drugs and toxins, reviewed by [6]. Some drugs require internalisation of the antibody to deliver the drug inside the cell, where it is then activated. Internalisation of a directly PE-conjugated T1-116C antibody by OCI-Ly8 human lymphoma cells was investigated. Target cells were incubated with T-116C, the OKT3 antibody (a negative control that lacks binding) and BB7.2 (positive control for HLA-A2, which is known to internalise) for 1-3 hours, after which externally bound antibody was stripped off and the cells were fixed and analysed by flow cytometry to detect the intracellular antibody (FIG. 15). HLA-A2 (BB7.2) internalisation was detectable within two hours of antibody incubation and T1-116C internalisation was detected within three hours of antibody incubation. These data demonstrate that there is the potential to use T1-116C as the tumour-targeting moiety in antibody drug conjugates.

Several TCRm antibodies against cancer targets have in vivo activity against tumours by mediating immune effector mechanisms such as complement-dependent cytotoxicity (CDC), antibody dependent phagocytosis (ADCP) and/or antibody-dependent cellular cytotoxicity (ADCC).[63-65]. The ability of a chimeric T1-116C antibody with a human IgG1 Fc domain to engage human immune effector cells was tested against B-cell lymphoma cell lines with high T1-116C binding, an isotype matched Rituximab antibody (anti-CD20) was used as a positive control (FIG. 16). The T1-116C antibody was able to engage immune effector cells to enable killing of both OCI-Ly1 and OCI-Ly8 B-cell lymphoma cell lines by ADCC and by ADCP, although less effectively than rituximab, with the highest dose (10 μg/ml) exhibiting the greatest effect. The CDC killing mediated by T1-116C, against OCI-Ly8 cells, was moderately higher than that achieved by rituximab at the two higher antibody concentrations.

The MDA-MB-231 cell line is derived from an aggressive triple receptor negative breast cancer, was labelled by T1-116C and had already been demonstrated to be targetable with a TCRm mAb against human chorionic gonadotropin beta (presented by HLA-A*0201) in vivo.[63] Recombinant T1-116C in either a human IgG1 (hlgG1) format or murine IgG2a (mlgG2a) format were tested for their ability to prevent the engraftment of MBA-MB-231 tumours in BALB/s nu/nu mice (10 mg/ml). The T1-116C IgG2a format antibody significantly inhibited tumour growth in vivo (P<0.0001) (FIG. 17). The human IgG1 format T1-116C antibody did not significantly affect tumour growth. Although hlgG1 can bind all activating murine FcγRs, it has been reported to be less potent than mlgG2a antibodies in mouse models,[66] which may contribute to the differences observed.

The T1-116C mlgG2a format antibody was further tested for its ability to prevent the growth of established MDA-MB-231 tumours in BALB/s nu/nu mice (10 mg/ml). Compared to an isotype matched control antibody (anti-fluorescein) or PBS carrier alone, the T1-116C antibody significantly reduced the growth rate of MDA-MB-231 tumours (P<0.0001).

Humanisation and de-immunisation of the T1-116C antibody was outsourced to Lonza Biologics plc. Screening did not identify any potentially high-risk post-translational modifications in the CDRs (complementarity determining regions) of antibody T1-116C. The optimal human germlines were identified, 3D computational models of the Fv domains were generated and Epibase™ profiling was used to design 16 humanised/deimmunised antibody variants (combinations of four light chain and four heavy chains, Table 6). A chimeric antibody containing the unaltered CDRs was also constructed.

TABLE 6
CDR sequences of humanised and
deimmunised T1-116C antibody variants.

| | CDR1 | CDR2 | CDR3 |
|---|---|---|---|
| VL | SASSSVSYIY | STSNLAS | QQRSNYPFT |
| Wildtype | (SEQ ID NO: 2) | (SEQ ID NO: 3) | (SEQ ID NO: 4) |
| VL_1 | SASSSVSYIY | STSNLAS | QQRSNYPFT |
| VL_2 | SASSSVSYIY | STSNLAS | QQRSNYPFT |
| VL_3 | SASSSVSYIY | 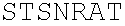 | QQRSNYPFT |
| | | (SEQ ID NO: 6) | |
| VL_4 | 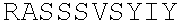 | 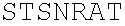 | QQRSNYPFT |
| | (SEQ ID NO: 5) | | |
| VH | NYWIH | NIYPSDSYSNYNQKFKD | SEGYYDYEAFDY |
| wildtype | (SEQ ID NO: 7) | (SEQ ID NO: 8) | (SEQ ID NO: 9) |
| VH_1 | NYWIH | NIYPSDSYSNYNQKFKD | SEGYYDYEAFDY |
| VH_2 | NYWIH | 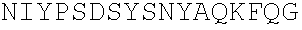 | SEGYYDYEAFDY |
| | | (SEQ ID NO: 11) | |
| VH_3 | NYWIH | 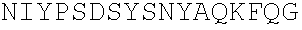 | SEGYYDYEAFDY |
| VH_4 | 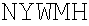 | 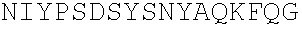 | SEGYYDYEAFDY |
| | (SEQ ID NO: 10) | | |

The humanised and deimmunised T1-116C variants were transiently expressed and their yield, levels of soluble aggregates and their binding affinity for the immunising tetramer (p53₆₅₋₇₃ peptide/HLA*0201 complex) are illustrated (Table 7). Only the four T1-116C variants containing the VL1 light chain were capable of binding their target antigen. Variants 1 and 2 showed comparable affinity to the parental and chimeric T1-116C antibody while variants 3 and 4 retained binding ability but at a lower affinity. These four variants demonstrated acceptable yields and low levels of aggregates.

TABLE 7
Summary of expression and binding data for transiently
expressed T1-116C humanisation variants

| | | | Conc. | Yield | % insoluble | |
|---|---|---|---|---|---|---|
| T1-116C Variant | Heavy chain | Light chain | (mg/ml) | (mg) | aggregate | K_{D}(μM) |
| T1-116C chimeric | T1-116C_VL | T1-116C_VH | | | | 1.25-1.76 |
| T1-116C variant 1 | T1-116C_VL_1 | T1-116C_VH_1 | 2.37 | 3.84 | 0.7 | 3.76 |
| T1-116C variant 2 | T1-116C_VL_1 | T1-116C_VH_2 | 8.30 | 3.36 | 0.8 | 5.32 |
| T1-116C variant 3 | T1-116C_VL_1 | T1-116C_VH_3 | 4.17 | 3.21 | 0.9 | 29.30 |
| T1-116C variant 4 | T1-116C_VL_1 | T1-116C_VH_4 | 8.23 | 3.87 | 0.9 | 108.0 |
| T1-116C variant 5 | T1-116C_VL_2 | T1-116C_VH_1 | 2.69 | 4.03 | 1.0 | — |
| T1-116C variant 6 | T1-116C_VL_2 | T1-116C_VH_2 | 3.11 | 1.87 | 0.7 | — |
| T1-116C variant 7 | T1-116C_VL_2 | T1-116C_VH_3 | 9.62 | 5.77 | 0.8 | — |
| T1-116C variant 8 | T1-116C_VL_2 | T1-116C_VH_4 | 5.03 | 2.56 | 0.9 | — |
| T1-116C variant 9 | T1-116C_VL_3 | T1-116C_VH_1 | 12.41 | 4.10 | 0.7 | — |
| T1-116C variant 10 | T1-116C_VL_3 | T1-116C_VH_2 | 15.56 | 4.36 | 0.7 | — |
| T1-116C variant 11 | T1-116C_VL_3 | T1-116C_VH_3 | 9.57 | 3.59 | 0.6 | — |
| T1-116C variant 12 | T1-116C_VL_3 | T1-116C_VH_4 | 13.94 | 3.90 | 1.0 | — |
| T1-116C variant 13 | T1-116C_VL_4 | T1-116C_VH_1 | 3.15 | 3.30 | 0.4 | — |
| T1-116C variant 14 | T1-116C_VL_4 | T1-116C_VH_2 | 16.07 | 4.58 | 0.8 | — |
| T1-116C variant 15 | T1-116C_VL_4 | T1-116C_VH_3 | 8.53 | 3.41 | 0.6 | — |
| T1-116C variant 16 | T1-116C_VL_4 | T1-116C_VH_4 | 19.24 | 5.58 | 1.0 | — |

The heavy and light chain variable domains for the antibodies were synthesised and cloned into Lonza's GS XCeed ™ vectors. Light chain variable domain encoding regions were transferred into pXC Kappa and heavy chain variable domain encoding regions into pXC IgG1f(ΔK) vectors respectively. Single gene vectors were transiently co-transfected into Chinese Hamster Ovary cells GS Knockout (CHOKSV GS-KO) alongside the reference chimeric antibody at 200 ml scale. Six days post-transfection, the clarified supernatant was purified by Protein A chromatography. Product quality and purity was assessed by SDS-PAGE and SE-HPLC and antigen binding affinity was measured by QCM.

The ability of the four humanised T1-116C antibody variants to recognise the p53₆₅₋₇₃ peptide/HLA*0201 complex on the surface of cells was tested by flow cytometry analysis in a T2 presentation assay and using a cancer cell line (FIG. 20). The T2 assay demonstrated effective binding of the chimeric T1-116C antibody and variants 1 and 2, with variants 3 and 4 showing less effective binding. All the antibodies retained their specificity for the p53₆₅₋₇₃ peptide and did not recognise the control Flu peptide. Staining of NCI-H1395 cells demonstrated good binding by variant 1 and 2, while variant 4 showed reduced binding and this was even lower with variant 3. In summary the T1-116C humanised variants 1 and 2 preferentially retain the desirable binding characteristics exhibited by the original murine antibody.

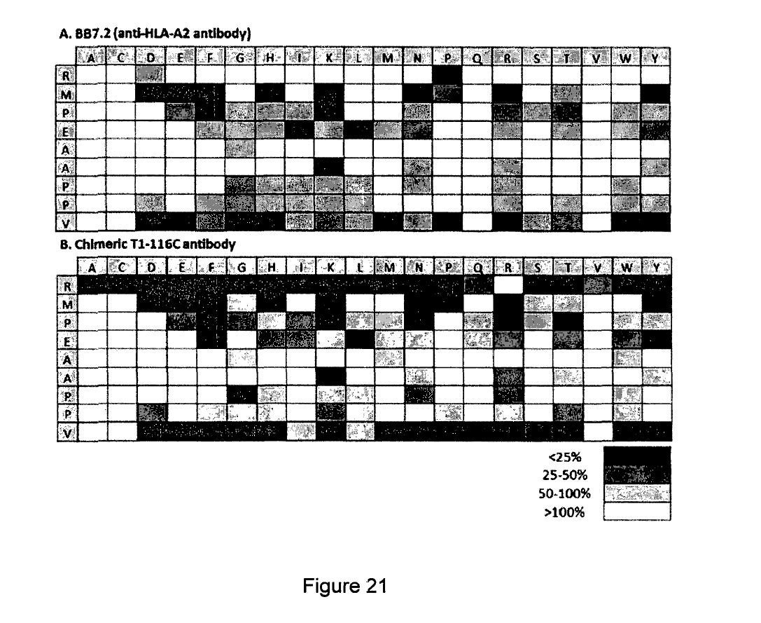

To further investigate the potential cross-activity of the T1-116C mAb, we expanded the replacement of individual amino acids to glycine or alanine, so that each position was individually changed to all possible 19 amino acids. The resulting 171 peptides were then compared to the original for their ability to be presented by HLA-A2 and to be bound by the T1-116C mAb in a T2 assay (FIG. 21). As observed previously the R at position 1 was required for T1-116C binding but not for HLA-A2 binding. Conservation of the anchor residues at position 2 and 9 was important for HLA-A2 presentation as demonstrated by HLA-A2 antibody BB7.2 binding (FIG. 21A), and the limited changes that retained HLA-A2 binding also retained antibody binding (FIG. 21B). Amino acids in the central region of the epitope did not significantly contribute to determining the specificity of T1-116C binding with all changes at position 5 retaining T1-116C binding (FIG. 21B).

Single Amino Acid Substitution T1-116C Consensus Sequence

| | |
|---|---|
| Position 1 | R |
| Position 2 | M, A, C, G, I, L, Q, S, T, V, W |
| Position 3 | P, A, C, D, H, L, M, Q, S, V, W, Y |
| Position 4 | E, A, C, D, G, K, M, N, P, Q, S, V |
| Position 5 | X |
| Position 6 | A, C, D, E, F, G, H, I, L, M, N, P, Q, S, T, V, W, Y |
| Position 7 | P, A, C, D, E, F, H, I, K, L, M, Q, S, T, V, W, Y |
| Position 8 | P, A, C, E, F, G, H, I, L, M, N, Q, R, S, V, W, Y |
| Position 9 | V, A, C, I, L |

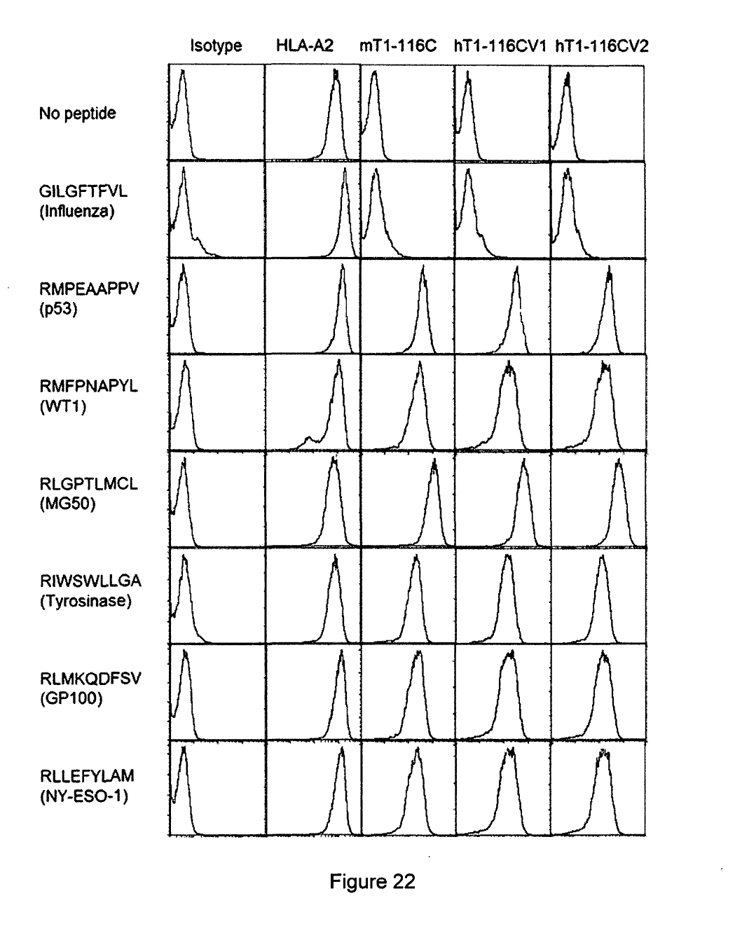

Peptides that retained T1-116C binding were used to interrogate the Immune Epitope Database (IEDB) that contains experimentally proven processed T-cell epitopes. We selected several peptides derived from known cancer antigens (including WT-1 and NY-ESO-1 peptides that displayed a single amino acid mismatch to the T1-116C consensus), and tested their binding to the original murine and humanised T1-116C mAbs variants 1 and 2 in a T2 assay. The following peptides were found to be recognised by T1-116C in an HLA-A2-dependent manner: MG501244-1252(RLGPTLMCL), Tyrosinase473-481 (RIWSWLLGA), gp100626-634 (RLMKQDFSV). Wilms Tumour protein 1 (WT1)126-134 (RMFPNAPYL), and NY-ESO-186-94(RLLEFYLAM) were also selected given their sequences were highly similar to the single amino acid substitution consensus sequence, and were additionally found to be recognised by T1-116C in an HLA-A2-dependent manner (FIG. 22).

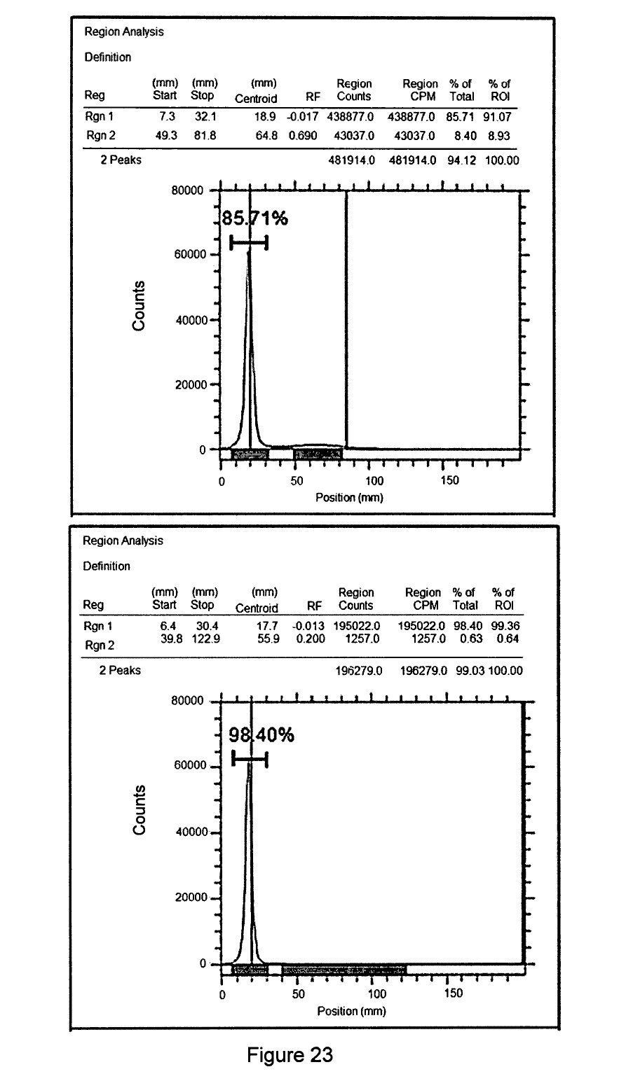

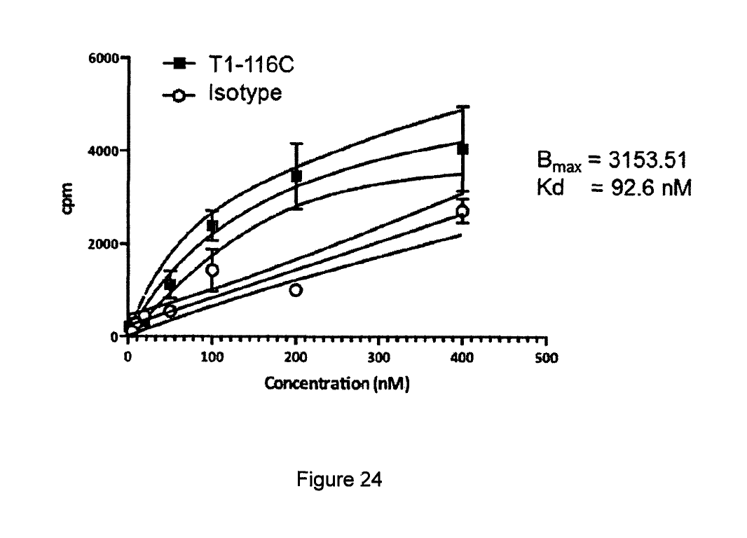

The T1-116C mAb was radiolabelled with 111In-chloride through p-SCN-Bn-DTPA) and purified by size exclusion chromatography (FIG. 23). An isotype mAb was processed similarly. To investigate the avidity of T1-116C antibody binding to tumour cells, MDA-MB-231 breast cancer cells were bound with a similarly labelled humanised 111In-T1-116C antibody at various concentrations and radioactive counts in cell lysates were measured to calculate the saturation binding. A Kd of 92.6 nM was obtained (FIG. 24), suggesting a strong binding avidity of T-116C to the cells and confirming that the antibody retained its binding activity after radiolabelling. The number of binding site calculated per cell (Bmax) of 3154 for the radiolabelled T1-116C antibody (FIG. 24) was similar to the 1956 determined for MDA-MB-231 using QuantiBRITE beads (Table 5).

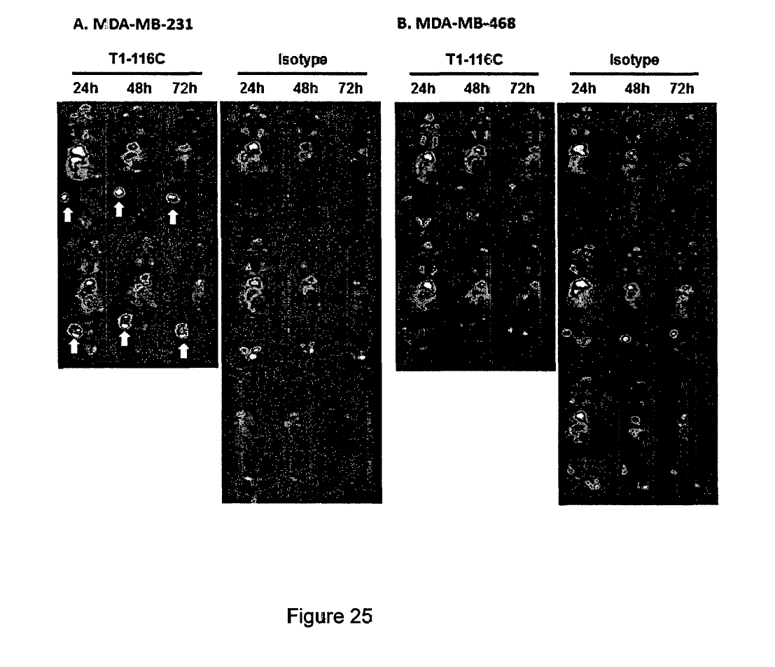

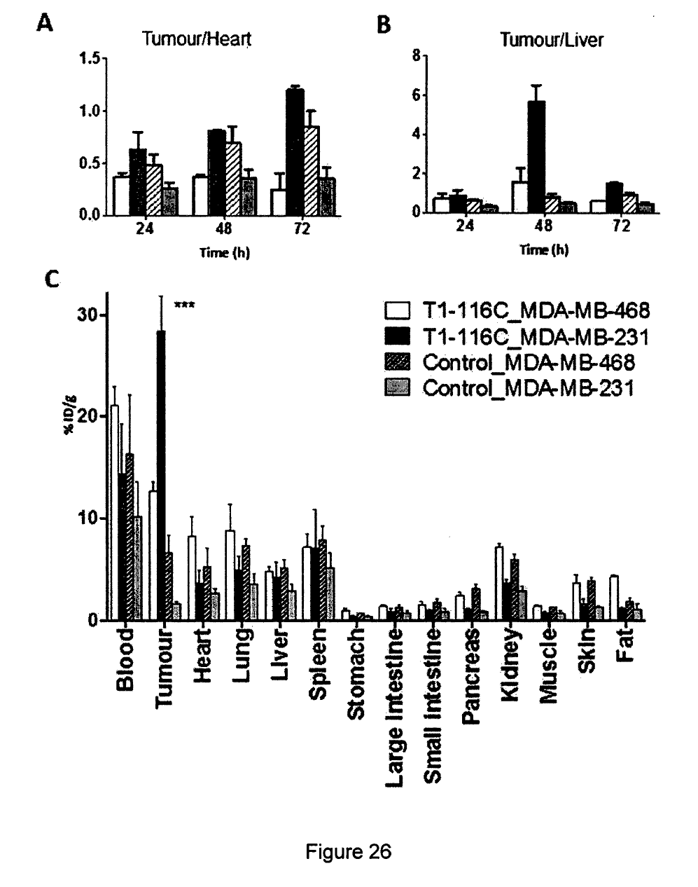

To investigate the in vivo bio-distribution of the T1-116C antibody, SPECT imaging was conducted at 24, 48, and 72 hours following administration of 111In-labelled T1-116C antibody (or an isotype control) in mouse xenografts derived from breast cancer cell lines MDA-MB-231 (HLA-A2+) and MDA-MB-468 (HLA-A2-) (FIGS. 25 and 26). Both T1-116C and the isotype control antibody followed the conventional pattern of uptake and clearance, showing initial enrichment in heart and liver and then later excretion through the bladder. The T1-116C mAb showed significant enrichment at the MDA-MB-231 tumour sites at all time points, which was not observed with MDA-MB-468 tumours lacking HLA-A2 expression or the isotype control antibody (FIG. 25). The organ distribution of the radiolabelled antibodies was investigated at the end of the experiment when organs were harvested by dissection and the radioactivity in each organ was measured (FIG. 26C). The T1-116C antibody was shown to have significantly higher enrichment in MDA-MB-231 tumour samples, but no significantly higher levels were found in other organs compared with an isotype control antibody.

These data illustrate that the T1-116C antibody can be used as an in vivo imaging agent. Imaging could be used to confirm the specificity of antibody binding in vivo and to stratify patients suitable for T1-116C therapy.

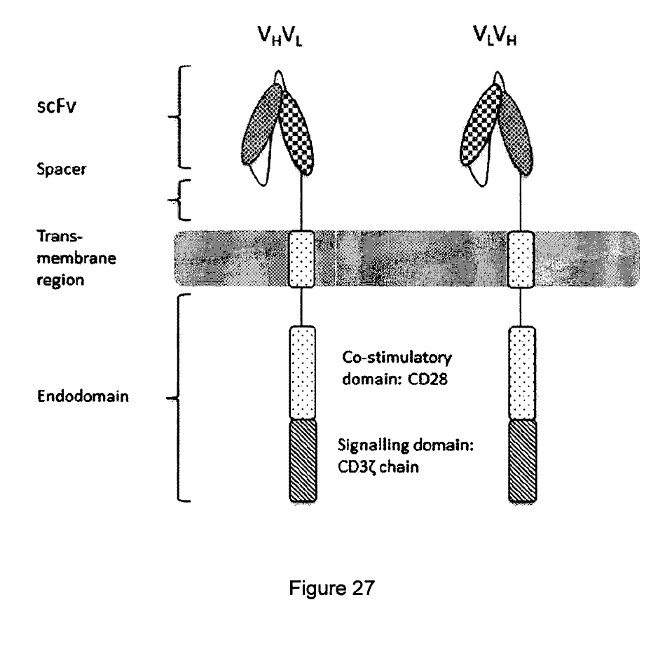

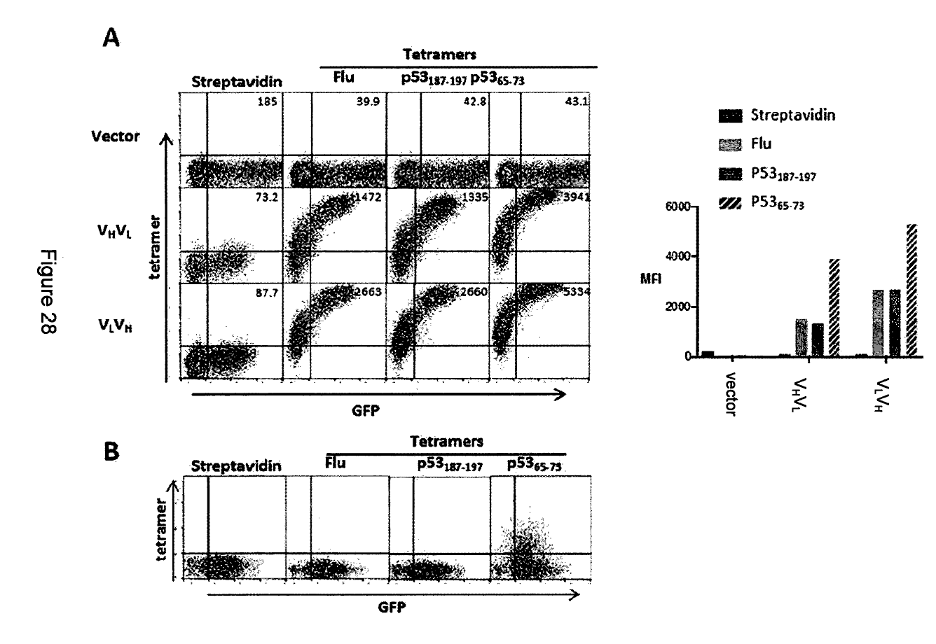

One of the major breakthroughs in cancer immunotherapy is the successful treatment of B-cell acute lymphoblastic leukaemia (B-ALL) using CD19 CAR T cells [76]. TCRm antibodies recognising cancer epitopes have potential as CAR T-cell targeting agents. To evaluate the suitability of the T1-116C antibody for this approach, two forms of CAR construct containing T1-116C variable regions in a second generation of CAR format, with alternative VH and VL orientations were generated (FIG. 27). Single chain variable fragments (scFv) of the T1-116C variable regions were presented on the cell surface by a CD28 stalk region, followed by the transmembrane and co-stimulatory region of CD28 and the signalling domain from the CD3 chain. The two T1-116C CAR constructs were transfected into HEK293T cells and tested for their ability to bind HLA-A2/p53₆₅₋₇₃ or control tetramers containing either Flu or an unrelated p53 peptide. While control HLA-A2 tetramers with irrelevant peptides bound to HEK293T cells transfected with either forms of CAR constructs, the p53₆₅₋₇₃ tetramers showed much higher binding in both cases (FIG. 28A). One explanation is that the CAR receptors exhibit some basal binding to HLA-A2, in addition to enhanced binding to the p53₆₅₋₇₃ epitope. HEK293T cells are derived from embryonic kidney. To investigate the specificity of the T1-116C CAR when expressed on T cells the VLVH format of CAR construct was used to transduce the Jurkat T-cell line. T1-116C CAR transduced Jurkat cells only exhibited binding to the p53₆₅₋₇₃ tetramer and not to controls tetramers (FIG. 28B). This suggests that the cell type used for CAR expression may affect the specificity of tetramer binding and indicates that the T1-116C antibody can retain specific binding when expressed in a single chain format on the cell surface.

### Sequences

Light chain sequences
T1-116C-VL
(SEQ ID NO: 12)
QIVLTQSPAIMSASPGEKVTVTCSASSSVSYIYWFQQKPGTSPKLWIYST
SNLASGVPARFSGSGSGTSYSLTISRMEAEDAATYYCQQRSNYPFTFGSG
TKLEIKR
T1-116C-VL_1
(SEQ ID NO: 13)
QIVLTQSPATLSLSPGERATLSCSASSSVSYIYWFQQKPGQAPRLWIYST
SNLASGVPARFSGSGSGTDYTLTISSLEPEDAAVYYCQQRSNYPFTFGSG
TKLEIKR
T1-116C-VL_2
(SEQ ID NO: 14)
QIVLTQSPATLSLSPGERATLSCSASSSVSYIYWYQQKPGQAPRLLIYST
SNLASGIPARFSGSGSGTDYTLTISSLEPEDFAVYYCQQRSNYPFTFGQG
TKLEIKR
T1-116C-VL_3
(SEQ ID NO: 15)
QIVLTQSPATLSLSPGERATLSCSASSSVSYIYWYQQKPGQAPRLLIYST
SNRATGIPARFSGSGSGTDYTLTISSLEPEDFAVYYCQQRSNYPFTFGQG
TKLEIKR
T1-116C-VL_4
(SEQ ID NO: 16)
QIVLTQSPATLSLSPGERATLSCRASSSVSYIYWYQQKPGQAPRLLIYST
SNRATGIPARFSGSGSGTDYTLTISSLEPEDFAVYYCQQRSNYPFTFGQG
TKLERKR
Heavy chain sequences
T1-116C-VH
(SEQ ID NO: 17)
EVQLQQSGAELVRPGASVKLSCKASGYTFTNYWIHWVKQRPGQGLEWIGN
IYPSDSYSNYNQKFKDKATLTVDRSSSTAYMQLSSPTSEDSAVYYCARSE
GYYDYEAFDYWGQGTTLTVSS
T1-116C-VH_1
(SEQ ID NO: 18)
EVQLVQSGAEVKKPGASVKVSCKASGYTFTNYWIHWVRQAPGQGLEWMGN
IYPSDSYSNYNQKFKDRATLTVDRSTSTAYMELSSLRSEDTAVYYCARSE
GYYDYEAFDYWGQGTLVTVSS
T1-116C-VH_2
(SEQ ID: 19)
QVQLVQSGAEVKKPGASVKVSCKASGYTFTNYWIHWVRQAPGQGLEWMGN
IYPSDSYSNYAQKFQGRVTLTVDTSTSTAYMELSSLRSEDTAVYYCARSE
GYYDYEAFDYWGQGTLVTVSS
T1-116C-VH_3
(SEQ ID NO: 20)
QVQLVQSGAEVKKPGASVKVSCKASGYTFTNYWIHWVRQAPGQGLEWMGN
IYPSDSYSNYAQKFQGRVTITADTSTSTAYMELSSLRSEDTAVYYCARSE
GYYDYEAFDYWGQGTLVTVSS
T1-116C-VH_4
(SEQ ID NO: 21)
QVQLVQSGAEVKKPGASVKVSCKASGYTFTNYWMHWVRQAPGQGLEWMGN
IYPSDSYTNYAQKFQGRVTITADTSTSTAYMELSSLRSEDTAVYYCARSE
GYYDYEAFDYWGQGTLVTVSS

The following materials and methods are those used by the present inventors in the above Example.

### Cell Culture

Cell lines were maintained in RPMI 1640 media supplemented with 10% fetal calf serum (FCS), 2 mM glutamine, and antibiotics (streptomycin [50 μg/mL] and penicillin [50 U/mL]) at 37° C. and 5% CO₂.

### Generation of HLA-A2/p53 Tetramer and Chimeric Tetramer

A bacterial expression construct encoding the human HLA-A*0201 extracellular domain (amino acids 24-293) fused with a C-terminal BirA biotinylation sequence (LNDIFEAQKIEWH), and separate construct expressing mature human β2 microglobulin β2m, amino acids 21-119), were each generated and transformed into competent Escherichia coli strain BL21(DE3). Protein expression was induced by addition of 0.5 mM IPTG in low-salt LB medium (1% Tryptone, 0.5% Yeast extract and 0.5% NaCl W/V), and insoluble inclusion bodies containing the recombinant proteins were purified using BugBuster (Merck), according to the manufacturer's instructions. Peptides were synthesised by the peptide synthesis facility in the Weatherall Institute of Molecular Medicine (University of Oxford), these included p53 peptide1 (amino acids 65-73, RMPEAAPPV), peptide 2 (amino acids 187-197 GLAPPQHLIRV), and a control peptide derived from influenza A virus M1 protein (GILGFVFTL).

HLA-A2 tetramers were generated as previously described (Altman 1996 Science 273: 94-96). Briefly, HLA-A*0201 (15 mg), (32m (12.5 mg) and peptide (5 mg) were added into 500 ml of refolding buffer (100 mM Tris.Cl pH8.0, 400 mM L-Arginine, 2 mM EDTA, 5 mM reduced-glutathione, 0.5 mM oxidised-glutathione, and 0.1 mM PMSF) and refolded for 48 h. The refolding complex was concentrated and buffer exchanged to 10 mM Tris.Cl pH8.0, before being biotinylated with BirA protein biotin ligase (Avidity LLC) according to the manufacturer's instructions. Biotinylated protein was then separated using an Akta Purifier FPLC with a Sephadex 75 column and HLA-A2/β2m/peptide monomers were isolated. Biotinylated monomers in FPLC buffer (20 mM Tris.Cl pH8.0, 150 mM NaCl) were aliquoted and stored at −80° C., and aliquots were thawed and tetramerised with Extravidin-PE or -APC (Sigma) on use. Tetramers are commonly described according to their HLA haplotype and peptide and the invariant (32m is not described but will be present in the complex.

To generate chimeric tetramers, a bacterial expression construct encoding a chimeric protein composed of human HLA-A2 α1α2 domain (amino acids 24-208, from the wildtype HLA-A*201 expression construct) and murine H-2Dd α3 domain (amino acids 185-274, cloned from BALB/c mouse spleen cells) was generated by overlapping PCR. In addition, cDNA sequence encoding mature murine (32m (amino acids 21-119) was cloned from BALB/c mouse spleen cells, and PCR site-directed mutagenesis was performed on murine (32m to include the following changes to improve its refolding efficiency with HLA-A*0201: Pro53Ser, His54Asp, Met71 His, Met74Leu. Protein expression, refolding and tetramerisation were performed similarly to human tetramers.

### Generation of Anti-p53 TCRm Monoclonal Antibodies

MF1 (used to generate T1-116C) and BALB/c mice (6-8 week old females) were immunised with the HLA-A*0201/p53 tetramers following a standard protocol, i.e., each mouse was given three immunisations, with 100 μg tetramer per immunisation, at 10 day intervals. Forty days post the first immunisation, a boost immunisation, 100 μg tetramer, was given and fusions were performed two days later. A standard fusion protocol was followed [67] with NS0 murine myeloma cells as the fusion partner and hybridomas were grown out under hypoxanthine, aminopterin and thymidine (HAT) selection.

Hybridoma supernatants were screened for the presence of secreted antibodies specifically, or preferentially, recognising the immunising tetramer containing a p53 peptide rather than a control tetramer containing a peptide from influenza virus, by ELISA. Wild type HLA-A*0201 tetramers with the immunising p53 peptide or influenza peptide were screened simultaneously and individual hybridoma colonies were picked from wells where the supernatant showed enhanced binding to a tetramer containing the immunising peptide rather than control peptide. Only individual colonies secreting antibodies that specifically recognised the tetramer containing the immunising peptide were studied further and were cloned by limiting dilution. All supernatants were screened for reactivity with a wild type human tetramer, even when the immunising tetramer was chimeric.

T1-116C antibody gene cloning and sequencing Total RNA was extracted from 5×10⁶ T1-116C hybridoma cells using an RNeasy kit (Qiagen), and first strand cDNA was synthesised using SuperScript III Reverse Transcriptase (Invitrogen) and Oligo(dT)₆ primer. The heavy chain variable region was amplified using the primers listed in Table 7, and light chain variable region was amplified using the primers listed in Table 8 (Brocks et al 2001 Molecular Medicine 7: 461-469). PCR products were purified with a Wizard® SV Gel and PCR Clean-Up System (Promega) and cloned into a TOPO vector using a Zero Blunt® TOPO® PCR Cloning Kit (Invitrogen). The cloned T1-116C sequences were then sequenced.

TABLE 7

| | |
|---|---|
| MHV.B1 | GATGTGAAGCTTCAGGAGTC |
| MHV.B2 | CAGGTGCAGCTGAAGGAGTC |
| MHV.B3 | CAGGTGCAGCTGAAGCAGTC |
| MHV.B4 | CAGGTTACTCTGAAAGAGTC |
| MHV.B5 | GAGGTCCAGCTGCAACAATCT |
| MHV.B6 | GAGGTCCAGCTGCAGCAGC |
| MHV.B7 | CAGGTCCAACTGCAGCAGCCT |
| MHV.B8 | GAGGTGAAGCTGGTGGAGTC |
| MHV.B9 | GAGGTGAAGCTGGTGGAATC |
| MHV.B10 | GATGTGAACTTGGAAGTGTC |
| MHV.B11 | GAGGTCCAGCTGCAACAGTC |
| MHV.B12 | GAGGTGCAGCTGGAGGAGTC |
| MHC.F | GCCAGTGGATAGTCAGATGGGGGTGTCGTTTTGGC |

TABLE 8

| | |
|---|---|
| MKV.B1 | GATGTTTTGATGACCCAAACT |
| MKV.B2 | GATATTGTGATGACGCAGGCT |
| MKV.B3 | GATATTGTGATAACCCAG |
| MKV.B4 | GACATTGTGCTGACCCAATCT |
| MKV.B5 | GACATTGTGATGACCCAGTCT |
| MKV.B6 | GATATTGTGCTAACTCAGTCT |
| MKV.B7 | GATATCCAGATGACACAGACT |
| MKV.B8 | GACATCCAGCTGACTCAGTCT |
| MKV.B9 | CAAATTGTTCTCACCCAGTCT |
| MKV.B10 | GACATTCTGATGACCCAGTCT |
| MKC.F | GGATACAGTTGGTGCAGCATC |

### Production of Purified Antibodies

Production of purified TCRm antibodies from hybridoma supernatant was achieved by culturing hybridoma cells in serum-free medium to extinction, or in CL350 bioreactors, followed by protein A or protein G purification of immunoglobulin.

Large-scale production of recombinant T1-116C antibody (mlgG1) and its isotype switching (mlgG2a or hlgG1) and endotoxin-free antibody production were outsourced to Absolute Antibody Ltd. Briefly, T1-116C heavy and light chains were cloned into pUV vectors, then transiently transfected into ABS293 cells. Culture supernatants were harvested and antibody purified through Protein A affinity chromatography. Purified antibody was analysed by SDS-PAGE and endotoxin level was determined by LAL chromogenic endotoxin assay.

For the QCM assay, a T1-116C human IgG1 chimeric antibody and sixteen humanised and deimmunised T1-116C variants were produced by Lonza Biologics PLC. Briefly, T1-116C heavy and light chains were cloned into Lonza's GS Xceed vectors, which were subsequently transiently transfected into CHOKISV GS-KO cells. Cell culture supernatant was harvested 6 days post transfection, filtered and antibody purified by Protein A chromatography. Purified material was analysed by SDS-PAGE and SE-HPLC.

### ELISA Assay

Ninety-six well MaxiSorp plates were coated with 100 μl of streptavidin at 10 μg/ml in PBS at 4° C. overnight. The plates were then washed with PBS/0.1% Tween-20 and blocked with 1% BSA in PBS for 2 h at room temperature. Plates were used fresh or kept at −20° C. after washing for future use.

To test hybridoma binding to HLA-A2/peptide complexes, plates were used fresh or recovered from the −20° C. freezer and thawed at room temperature. HLA-A2/peptide monomers were added to the wells at 1 μg/ml (100 μl) and incubated for 1 h at room temperature. After washing, 100 μl of mAb at 10 μg/ml or neat hybridoma supernatants were added to the wells and incubated for 1 h before washed. HRP conjugated anti-mouse secondary antibody was added at 1:1000 dilution to each well and incubated for 1 h. Substrate ABTS Solution (Roche) was added to each well (100 μl) after washing and OD405 nm was measured with a plate reader within 5-30 min.

### T2 Cell Binding Assay

Hybridoma supernatants and/or purified antibodies were further screened for their ability to recognise their target peptide (or potentially cross reactive peptides identified by peptide scanning) presented on the cell surface of peptide-pulsed T2 cells by HLA-A2.

Peptides were synthesised by the peptide synthesis facility in the Weatherall Institute of Molecular Medicine, University of Oxford (20 mg scale), or by Sigma-Aldrich (1 mg scale). TAP-deficient T2 cells cultured at logarithmic phase were pulsed with peptides at 100 mM (or a range of lower concentrations for peptide titration experiments) for 12 h in a U-shaped bottom 96 well tissue culture plate. Cell were then harvested and stained with TCRm antibodies and/or HLA-A2-specific mAb BB7.2 (Abcam), followed by APC conjugated goat anti-mouse secondary antibody (eBioscience). Samples were washed with FACS wash buffer (2% FBS in PBS+0.1% sodium azide) then fixed with 1% paraformaldehyde (in PBS) and acquired with a FACSCalibur (BD Bioscience).

### FACS Analysis

Cells were stained with either purified TCRm antibodies, generally at 10 μg/ml diluted in FACS wash buffer (2% FBS in PBS+0.1% sodium azide), or neat hybridoma supernatants, followed by the indicated allophycocyanin (APC)-conjugated secondary antibody at 1:200 dilution: for murine primary antibodies the secondary antibody was goat anti-mouse-APC (eBioscience); for humanised and chimeric antibodies the secondary antibody was goat anti-human IgG (H+L) secondary antibody (Jackson ImmunoResearch Laboratories). After washing, samples were fixed with 1% paraformaldehyde (in PBS) and acquired with a FACSCalibur (BD Bioscience). FACS data were analysed with FlowJo software (TreeStar Inc.). On some occasions a directly PE-conjugated T1-116C mAb (conjugation of the T1-116C was outsourced to BioLegend) was used.

### Immunocytochemistry

Acetone-fixed cytocentrifuge cell preparations of either peptide pulsed T2 cells (p53₆₅₋₇₃, no peptide or Flu peptide), or cancer cell lines, were incubated in a solution of hydrogen peroxide (0.3% H₂O₂, 0.1% NaN₃ in PBS) for 10 minutes to block any endogenous peroxidase activity. Slides were washed once in PBS then once in PBS-Tween (0.05% v/v, 3 minutes each wash). Slides were then incubated with primary antibody (T1-116C purified antibody, anti-HLA-A2 antibody clone BB7.2, mouse anti-p53 clone D01, Santa Cruz Biotechnology, sc-126 or isotype-matched control antibody (all at 10 μg/ml)) for 30 minutes. After washing as above, the slides were incubated in secondary antibody reagent (Dako REAL™ EnVision™ Detection System, K5007) for 30 minutes. Labelling was visualized using the Liquid DAB+ Substrate Chromogen System (Dako, K3468), allowing colour to develop for 10 minutes. After washing as above, cells were counter-stained with Gill 3 Hematoxylin (Thermo Scientific, 6765009) and coverslips were mounted using Aquatex (VWR 1.08562.0050). All steps were performed at room temperature.

### Western Blotting

Whole cell lysates were prepared using Mammalian Protein Extraction Reagent Thermo Scientific, 78503) containing a nuclease to degrade any nucleic acids and additional protease and phosphatase inhibitors. Protein concentrations were quantified using BCA assay (Thermo Scientific 23227). 30 μg whole cell lysates were resolved on 10% polyacrylamide gels and transferred to Protran™ nitrocellulose membranes (GE Healthcare, 15269794). Membranes were blocked in 5% (w/v) low fat milk in PBS for 1 hour at RT, and were then incubated with primary antibodies overnight at 4° C. diluted in 5% (w/v) low fat milk in PBS (mouse anti-p53 (D01, Santa Cruz Biotechnology, sc-126, 1 μg/ml), mouse anti-p53 (D07, Santa Cruz Biotechnology, sc-47698, 1 μg/ml), mouse anti-p53 (Pab1801, Santa Cruz Biotechnology, sc-98, 1 μg/ml), mouse anti-β-Actin (Sigma, clone AC-15) 1:20,000). This was followed by washing of the membranes in PBS (three washes) and PBS-Tween (0.1% v/v, one wash) at RT (5 minutes each wash) then incubation in secondary antibody solution (goat anti-mouse IgG-HRP, Dako, P0447, diluted in 5% w/v low fat milk in PBS) for 1 hour at RT. After washing as above, antibody binding was visualized with ECL reagent (GE Healthcare, RPN2106) and visualised with a G:BOX ChemiXRQ imaging system (Syngene).

### Real Time-Quantitative PCR (RT-qPCR)

Expression of transcripts was assessed using cDNAs from normal human tissue (Clontech Human MTC Panels I and II, Takara Bio Europe, 636742 and 636743), and a panel of malignant cell lines. MTC Panel cDNAs were prepared from pools of individual donors. Cell line total RNA was isolated using QIAgen's RNeasy Mini kit (QIAgen, 74106) according to the manufacturer's instructions (incorporating the DNase I treatment). 1 μg total RNA was then reverse transcribed using Superscript® μl (Life Technologies, 18080044). Cell line cDNAs were diluted 1/5 and 4 μl of diluted cDNA was used for qPCR (20 μl reaction volume). qPCR was performed using EXPRESS qPCR Supermix, Universal (Life Technologies, 11785-200) in a 96-well plate format on an MJ Research Chromo4 thermal cycler. Exon-spanning TaqMan® assays (Life Technologies) for target genes were selected to ensure widest transcript coverage and/or detection of transcripts containing (or adjacent to) the exon encoding a potentially T1-116C cross reactive peptide. Assay details are listed in Table 9. Normal tissue expression was normalized to β2M (Beta-2-microglobulin) and GAPDH (glyceraldehyde-3-phophate dehydrogenase), whilst cell line expression was normalised to TBP (TATA Box Binding Protein), 18S RNA and HPRT1 (Hypoxanthine phosphoribosyltransferase 1). Expression is presented relative to positive control samples.

TABLE 9

| Target Gene | TaqMan ® Assay |
|---|---|
| p53 | Hs01034249_m1 (Ex 10-11) |
| | Hs01034253_m1 (Ex 4-5) |
| UBR3 | Hs00419575_m1 (Ex 23-24) |
| | Hs01118905_m1 (Ex 36-37) |
| SHANK1 | Hs01061105_m1 (Ex 21-22) |
| | Hs01061106_m1 (Ex 22-23) |
| BSN | Hs01109152_m1 (Ex 3-4) |
| | Hs00185720_m1 (Ex 5-6) |
| AP3B1 | Hs00186383_m1 (Ex 2-3) |
| | Hs01564196_m1 (Ex 16-17) |
| LHX6 | Hs01030943_m1 (Ex 5-6) |
| | Hs01030946_m1 (Ex 8-9) |
| PANX2 | Hs00364525_m1 (Ex 1-2) |
| β2M | 4326319E |
| GAPDH | 4310884E |
| TBP | 4326322E |
| 18S RNA | 4319413E |
| HPRT1 | Hs99999909_m1 |

### Quartz Crystal Microbalance (QCM) Analysis

To determine the binding affinity of T1-116C mAb and its chimeric and humanised variants to HLA-A2/p53 peptide 1 monomers, a QCM assay was performed by Lonza Biologics plc. Briefly, a polyclonal rabbit anti-human IgG antibody (Attana) was immobilised onto an LNB Carboxyl Sensor Chip (Attana) via amine coupling, and T1-116C chimeric antibody or a negative control hlgG1 mAb was captured onto the chip at a concentration of 5.0 μg/ml (150 s contact time and 60 s association time at 10 μl/min). HLA-A2/p53 monomer titrations were performed in an 8 point 2-fold serial dilution (from 50 μg/ml, 1.08 μM) and injected over the surface for a contact time of 60 s in duplicates, and association was monitored for 300 s. Between each antibody injection, the surface was regenerated by one injection of 100 mM HCl (60 s contact time) followed by a subsequent injection of 20 mM NaOH (60 s contact time).

All measurements were performed at 25° C. in 1×HBS-T running buffer. Data was collected by Attester software v3.0 and subsequently processed in Attester Evaluation v3.0 and Clamp XP software to achieve kinetic data where data were fitted to a ‘Simple 1:1 Binding Model’.

### Quantitation of Antibody Molecules Bound Per Target Cell

Cell lines or T2 cells pulsed with the p53₆₅₋₇₃ peptide at 0.5-100 μM concentrations or the Flu peptide at 100 μM were stained with PE-conjugated T1-116C mAb (mAb:PE=1:1) or an isotype matched control antibody at 10 μg/ml for 30 min on ice. Cells were washed with FACS Wash buffer then fixed with 1% paraformaldehyde before being analysed with a FACSCalibur (BD Biosciences). QuantiBRITE-PE beads (BD Biosciences) were acquired in parallel and correlation between geometric means (corrected to remove background binding to isotype control antibody) and PE molecules/beads of the four QuantiBRITE bead populations was established according to the manufacturer's instructions. Numbers of T1-116C-PE antibody molecules bound per cell was calculated based on the correlation formula and subtraction of background from negative cells (unpulsed T2 cells [531 antibody molecules bound] or 293T cells [437 antibody molecules bound]).

### Antibody Internalisation Assay

Human B cell lymphoma OCI-Ly8 cells were stained with PE-conjugated T1-116C mAb (10 μg/ml) at 4° C. for 20 min and washed with FACS wash buffer. Aliquots of the cells were fixed with 1% paraformaldehyde (to demonstrate cell surface labelling) and the rest were incubated at 37° C. for various time points to allow the labelled T1-116C antibody to internalise before the cells were harvested and stripped of externally bound antibody with a stripping buffer (150 mM NaCl, pH2.5) before being fixed with 1% paraformaldehyde. Samples were analysed by FACS. Antibodies OKT3-PE and BB7.2-PE served as negative (no binding to target cells) and positive (known to internalise) controls for internalisation.

### Complement Dependent Cytotoxicity (CDC) Assay

1×10⁵ cells were opsonised with antibody for 15 min at room temperature (RT) in a flat-bottomed 96-well plate. Human serum was added to a final volume of 10% and incubated for 30 min at 37° C. Cells were transferred to a FACS tube where 10 μL propidium iodide (P1) solution (10 μg/mL in PBS) was added prior to data acquisition. Percentage cell death was defined as the percentage PI+ cells of the total cell population.

### Antibody Dependent Cellular Phagocytosis (ADCP) Assay:

Mouse bone marrow derived macrophages (BMDM) were differentiated from the bone marrow of WT BALB/c female mice and cultured for 7-10 days in the presence of 20% L929 conditioned media (containing M-CSF). 5×10⁴ BMDM per well were plated in a flat-bottomed 96-well plate the day before the assay was performed as previously described.[68]

In brief, target cells were labelled with Carboxyfluorescein succinimidyl ester (CFSE) at RT before being washed once in RPMI media. The CFSE labelled cells were opsonised with antibody for 30 min at 4° C., washed once and then 2.5×10⁵ opsonised target cells added to the BMDM and left to co-culture at 37° C. for 1 hr. The BMDM were labelled with anti-F4/80-APC (Serotec) and the wells washed with PBS, before removal and analysis of the cells on FACS Calibur (BD Biosciences). Percentage phagocytosis was defined as the percentage of CFSE⁺ F4/80⁺ cells of the total F4/80⁺ population.

### Antibody Dependent Cellular Cytotoxicity (ADCC) Assay:

Human peripheral blood mononuclear cells (PBMC) were isolated by density gradient centrifugation. Target cells were labelled with calcein AM (Life Technologies) and suspended in RPMI. The labelled cells were opsonised with antibody for 30 min at 4° C. before washing once in RPMI media. The target cells and PBMC effector cells were co-cultured at a 50:1 (Effector:Target) ratio for 4 hr at 37° C. The cells were pelleted by centrifugation (1500 rpm for 5 min), the supernatant transferred to a white 96-well plate, and read using a Varioskan Flash (Thermo Scientific) to record calcein release (excitation wavelength 485 nm; emission wavelength 530 nm). Percent of maximum lysis was defined as the calcein release compared to the response recorded when cells were treated with 4% Triton-X100 solution.

### Xenograft Experiments

MDA-MB-231 cells (1×10⁷) in 100 μl Matrigel were injected subcutaneously into the flank of BALB/c nu/nu mice (n=10 per group). T1-116C in two formats, a murine IgG2a isotype (mlgG2a) versus a human IgG1 isotype (hlgG1), or PBS carrier alone, was administered twice a week (10 mg/kg for Ab and 200 μl for PBS) starting from the time of tumour inoculation. Tumour sizes were calculated as length×width×height×π/6. Geometric Mean Diameter (GMD) was calculated as (L×W×H)^{1/3}.

### T1-116C mAb Humanisation and Deimmunisation

T1-116C mAb was humanised and deimmunised by Lonza Biologics. Briefly, in silico humanisation and deimmunisation were performed on heavy and light chain sequences using CDR grafting technology and T-cell epitope reduction. Heavy chain and light chain variable region cDNAs were synthesised and cloned into expression vectors encoding human IgG1 framework. In total 16 variants were generated in addition to a chimeric format in which the murine VH-C1 fused with human IgG1-C2-C3 was paired with murine VL-CL. Transient transfection was performed in CHOK1SV GS-KO cells and humanised antibodies were purified from 200 ml culture supernatants via Protein A chromatography.

### Antibody Radiolabelling

T1-1160-mlgG2a and an isotype control antibody (Absolute Antibody Ltd, UK) were radiolabeled with 111In as previously described [77]. Briefly, 500 μg of T1-1160 or isotype control antibody was dissolved in 0.1 M sodium bicarbonate aqueous buffer (pH 8.2) before adding a 20-fold molar excess of 2-(4-isothiocyanatobenzyl)-diethylenetriaminepentaacetic acid (p-SCN-Bn-DTPA; Macrocyclics) and incubating for 1 h at 37° C. The DTPA-conjugated antibody was subsequently purified using a Sephadex G50 gel filtration column and radiolabeled using 111In-chloride (1 MBq per 1 μg of IgG). The protein was further purified by Sephadex G50 size exclusion chromatography. Radiochemical purity was determined by instant thin layer chromatography (iTLC) as >95%.

### Saturation Binding Assay

MDA-MB-231 breast cancer cells growing in 12-well plates were incubated with 111In-T1-116C antibody (1 MBq/μg) at various concentrations (2-400 nM) at 4° C. for 2 h. Cells were washed, lysed and the amount of cell-associated radioactivity was measured using an automated gammacounter. A saturation binding curve was fitted to the data using the GraphPad Prism software package to estimate the affinity (KD) and number of binding sites per cell (Bmax).

### In Vivo Imaging and Biodistribution

Female BALB/c nu/nu mice (Charles Rivers) were injected subcutaneously on their flanks with 1×106 MDA-MB-231 or MDA-MB-468 breast cancer cells. 111In-labelled T1-116C or mlgG2a isotype control antibody (5 MBq, 5 μg) was administered i.v. when tumour sizes reach 120 mm3 at day 20, and SPECT/CT imaging was performed at 24, 48, 72 h after injection, using a Bioscan NanoSPECT/CT. Volume-of-interest analysis was performed on SPECT images using the Inveon Research Workplace software package (Siemens). After imaging at 72 h post injection, animals were sacrificed and selected organs were removed, rinsed, blot dried, weighed, and the amount of 111In in each tissue was measured using an automated gammacounter. Uptake of 111In was expressed as the percentage of the injected dose per gram of tissue (% ID/g).

### Generation of Cell Lines Expressing T1-116C CARs

CAR constructs containing the T1-116C scFv in two different variable fragment orientations were generated according to the design in FIG. 27. The calcium phosphate transfection method was used to transiently transfect 293T cells with each T1-116C CAR construct. VSV-G, pLP1 and pLP2 plasmids were used for the lentiviral packaging (ViraPower™ kit, Invitrogen). The cells were incubated for 72 hours. Then the culture supernatants were transferred to Ultraclear ultracentrifuge tubes (Beckman) and supplemented with complete RPMI medium to 38 ml. The tubes were centrifuged at 28,000 rpm in a Beckman Optima L-90K ultracentrifuge using a SW28 rotor (Beckman Coulter) for 3 hours. The supernatants were discarded and the pellet re-suspended with the backflow. 0.5×106 Jurkat cells were harvested and re-suspended with the virus particles and were incubated in a CO2 incubator for 1 hour at 37° C. The cells were then supplemented with 10 ml of complete RPMI medium and cultured for 3 days. The cells were subsequently harvested and washed 10 times with complete RPMI, over a period of 1 week to become virus-free. The CAR-transduced cells were then sorted using a FACS sorter (FACSAria III, BD Biosciences) and subsequently cultured in complete RPMI for further expansion.

### REFERENCES

1. Bronte, G., et al., Monoclonal antibodies for the treatment of non-haematological tumours: update of an expanding scenario. Expert Opin Biol Ther, 2015. 15(1): p. 45-59.
2. Maloney, D. G., et al., IDEC-C2B8 (Rituximab) anti-CD20 monoclonal antibody therapy in patients with relapsed low-grade non-Hodgkin's lymphoma. Blood, 1997. 90(6): p. 2188-95.
3. Baselga, J., et al., Recombinant humanized anti-HER2 antibody (Herceptin) enhances the antitumor activity of paclitaxel and doxorubicin against HER2/neu overexpressing human breast cancer xenografts. Cancer Res, 1998. 58(13): p. 2825-31.
4. Rock, K. L., I. A. York, and A. L. Goldberg, Post-proteasomal antigen processing for major histocompatibility complex class I presentation. Nat Immunol, 2004. 5(7): p. 670-7.
5. Weidanz, J. A., et al., TCR-like biomolecules target peptide/MHC Class I complexes on the surface of infected and cancerous cells. Int Rev Immunol, 2011. 30(5-6): p. 328-40.
6. Dahan, R. and Y. Reiter, T-cell-receptor-like antibodies-generation, function and applications. Expert Rev Mol Med, 2012. 14: p. e6.
Cohen, M. and Y. Reiter, T-cell receptor-like antibodies: targeting the intracellular proteom therapeutic potential and clinical applications. Antibodies, 2013. 2: p. 517-534.
8. Cohen, C. J., et al., Recombinant antibodies with MHC-restricted, peptide-specific, T-cell receptor-like specificity: new tools to study antigen presentation and TCR-peptide-MHC interactions. J Mol Recognit, 2003. 16(5): p. 324-32.
9. Verma, B., et al., TCR mimic monoclonal antibodies induce apoptosis of tumor cells via immune effector-independent mechanisms. J Immunol, 2011. 186(5): p. 3265-76.
10. Brown, C. J., et al., Awakening guardian angels: drugging the p53 pathway. Nat Rev Cancer, 2009. 9(12): p. 862-73.
11. Malkin, D., Li-fraumeni syndrome. Genes Cancer, 2011. 2(4): p. 475-84.
12. Barnes, D. M., et al., Abnormal expression of wild type p53 protein in normal cells of a cancer family patient. Lancet, 1992. 340(8814): p. 259-63.
13. Vogelstein, B., D. Lane, and A. J. Levine, Surfing the p53 network. Nature, 2000. 408(6810): p. 307-10.
14. Khoo, K. H., C. S. Verma, and D. P. Lane, Drugging the p53 pathway: understanding the route to clinical efficacy. Nat Rev Drug Discov, 2014. 13(3): p. 217-36.
15. Freed-Pastor, W. A. and C. Prives, Mutant p53: one name, many proteins. Genes Dev, 2012. 26(12): p. 1268-86.
16. Garcia, P. B. and L. D. Attardi, Illuminating p53 function in cancer with genetically engineered mouse models. Semin Cell Dev Biol, 2014. 27: p. 74-85.
17. Hong, B., et al., Targeting tumor suppressor p53 for cancer therapy: strategies, challenges and opportunities. Curr Drug Targets, 2014. 15(1): p. 80-9.
18. Muller, P. A. and K. H. Vousden, Mutant p53 in cancer: new functions and therapeutic opportunities. Cancer Cell, 2014. 25(3): p. 304-17.
19. Theobald, M., et al., The sequence alteration associated with a mutational hotspot in p53 protects cells from lysis by cytotoxic T lymphocytes specific fora flanking peptide epitope. J Exp Med, 1998. 188(6): p. 1017-28.
20. Vierboom, M. P., et al., High steady-state levels of p53 are not a prerequisite for tumor eradication by wild-type p53-specific cytotoxic T lymphocytes. Cancer Res, 2000. 60(19): p. 5508-13.
21. Kuckelkorn, U., et al., The effect of the interferon-gamma-inducible processing machinery on the generation of a naturally tumor-associated human cytotoxic T lymphocyte epitope within a wild-type and mutant p53 sequence context. Eur J Immunol, 2002. 32(5): p. 1368-75.
22. De Leo, A. B., p53-based immunotherapy of cancer. Approaches ro reversing unresponsiveness to T lymphocytes and preventing tumor escape. Adv Otorhinolaryngol, 2005. 62: p. 134-50.
23. Labrecque, S., et al., Analysis of the anti-p53 antibody response in cancer patients. Cancer Res, 1993. 53(15): p. 3468-71.
24. Yanuck, M., et al., A mutant p53 tumor suppressor protein is a target for peptide-induced CD8+ cytotoxic T-cells. Cancer Res, 1993. 53(14): p. 3257-61.
25. Humar, M., et al., DNA vaccination with a mutated p53 allele induces specific cytolytic T cells and protects against tumor cell growth and the formation of metastasis. J Cancer Res Clin Oncol, 2009. 135(4): p. 567-80.
26. Noguchi, Y., Y. T. Chen, and L. J. Old, A mouse mutant p53 product recognized by CD4+ and CD8+ T cells. Proc Natl Acad Sci USA, 1994. 91(8): p. 3171-5.
27. Theobald, M. and R. Offringa, Anti-p53-directed immunotherapy of malignant disease. Expert Rev Mol Med, 2003. 5(11): p. 1-13.
28. DeLeo, A. B. and T. L. Whiteside, Development of multi-epitope vaccines targeting wild-type sequence p53 peptides. Expert Rev Vaccines, 2008. 7(7): p. 1031-40.
29. Vermeij, R., et al., Immunological and clinical effects of vaccines targeting p53-overexpressing malignancies. J Biomed Biotechnol, 2011. 2011: p. 702146.
30. Choi, J. and L. A. Donehower, p53 in embryonic development: maintaining a fine balance. Cell Mol Life Sci, 1999. 55(1): p. 38-47.
31. Rogel, A., et al., p53 cellular tumor antigen: analysis of mRNA levels in normal adult tissues, embryos, and tumors. Mol Cell Biol, 1985. 5(10): p. 2851-5.
32. Kubbutat, M. H. and K. H. Vousden, Keeping an old friend under control: regulation of p53 stability. Mol Med Today, 1998. 4(6): p. 250-6.
33. Milner, J., Different forms of p53 detected by monoclonal antibodies in non-dividing and dividing lymphocytes. Nature, 1984. 310(5973): p. 143-5.
34. Pezzella, F., et al., Antibody for detecting p53 protein by immunohistochemistry in normal-tissues. Journal Of Clinical Pathology, 1994. 47: p. 592-596.
35. Pillai, G., et al., p53 expression in normal paraffin-embedded tissue using different antibodies and antigen retrieval buffer systems. Histopathology, 2003. 42(1): p. 83-7.
36. Nijman, H. W., et al., Immunologic aspect of ovarian cancer and p53 as tumor antigen. J Transl Med, 2005. 3: p. 34.
37. Vierboom, M. P., et al., Tumor eradication by wild-type p53-specific cytotoxic T lymphocytes. J Exp Med, 1997. 186(5): p. 695-704.
38. Zwaveling, S., et al., Antitumor efficacy of wild-type p53-specific CD4(+) T-helper cells. Cancer Res, 2002. 62(21): p. 6187-93.
39. Theoret, M. R., et al., Relationship of p53 overexpression on cancers and recognition by anti-p53 T cell receptor-transduced T cells. Hum Gene Ther, 2008. 19(11): p. 1219-32.
40. Cheever, M. A., et al., The prioritization of cancer antigens: a national cancer institute pilot project for the acceleration of translational research. Clin Cancer Res, 2009. 15(17): p. 5323-37.
41. Molloy, P. E., A. K. Sewell, and B. K. Jakobsen, Soluble T cell receptors: novel immunotherapies. Curr Opin Pharmacol, 2005. 5(4): p. 438-43.
42. Stone, J. D., et al., T cell receptor engineering. Methods Enzymol, 2012. 503: p. 189-222.
43. Laugel, B., D. A. Price, and A. K. Sewell, On the path to TCR-directed therapeutics. Nat Biotechnol, 2006. 24(6): p. 615-6.
44. Foote, J. and H. N. Eisen, Breaking the affinity ceiling for antibodies and T cell receptors. Proc Natl Acad Sci USA, 2000. 97(20): p. 10679-81.
45. Holler, P. D., et al., In vitro evolution of a T cell receptor with high affinity for peptide/MHC. Proc Natl Acad Sci USA, 2000. 97(10): p. 5387-92.
46. Liddy, N., et al., Monoclonal TCR-redirected tumor cell killing. Nat Med, 2012. 18(6): p. 980-7.
47. Oren, R., et al., Functional comparison of engineered T cells carrying a native TCR versus TCR-like antibody-based chimeric antigen receptors indicates affinity/avidity thresholds. J Immunol, 2014. 193(11): p. 5733-43.
48. Epel, M., et al., A functional recombinant single-chain T cell receptor fragment capable of selectively targeting antigen-presenting cells. Cancer Immunol Immunother, 2002. 51(10): p. 565-73.
49. Card, K. F., et al., A soluble single-chain T-cell receptor IL-2 fusion protein retains MHC-restricted peptide specificity and IL-2 bioactivity. Cancer Immunol Immunother, 2004. 53(4): p. 345-57.
50. Zhu, X., et al., Visualization of p53(264-272)/HLA-A*0201 complexes naturally presented on tumor cell surface by a multimeric soluble single-chain T cell receptor. J Immunol, 2006. 176(5): p. 3223-32.
51. Fishman, M. N., et al., Phase I trial of ALT-801, an interleukin-2/T-cell receptor fusion protein targeting p53 (aa264-272)/HLA-A*0201 complex, in patients with advanced malignancies. Clin Cancer Res, 2011. 17(24): p. 7765-75.
52. Mosquera, L. A., et al., In vitro and in vivo characterization of a novel antibody-like single-chain TCR human IgG1 fusion protein. J Immunol, 2005. 174(7): p. 4381-8.
53. Hoffmann, T. K., et al., Frequencies of tetramer+ T cells specific for the wild-type sequence p53(264-272) peptide in the circulation of patients with head and neck cancer. Cancer Res, 2002. 62(12): p. 3521-9.
54. Theobald, M., et al., Tolerance to p53 by A2.1-restricted cytotoxic T lymphocytes. J Exp Med, 1997. 185(5): p. 833-41.
55. Barfoed, A. M., et al., Cytotoxic T-lymphocyte clones, established by stimulation with the HLA-A2 binding p5365-73 wild type peptide loaded on dendritic cells In vitro, specifically recognize and lyse HLA-A2 tumour cells overexpressing the p53 protein. Scand J Immunol, 2000. 51(2): p. 128-33.
56. Wurtzen, P. A., et al., Specific killing of P53 mutated tumor cell lines by a cross-reactive human HLA-A2-restricted P53-specific CTL line. Int J Cancer, 2001. 93(6): p. 855-61.
57. Salter, R. D. and P. Cresswell, Impaired assembly and transport of HLA-A and-B antigens in a mutant TxB cell hybrid. EMBO J, 1986. 5(5): p. 943-9.
58. MacCallum, D. E., et al., The p53 response to ionising radiation in adult and developing murine tissues. Oncogene, 1996. 13(12): p. 2575-87.
59. Dainiak, N., Hematologic consequences of exposure to ionizing radiation. Exp Hematol, 2002. 30(6): p. 513-28.
60. Maas, K., et al., Reduced p53 in peripheral blood mononuclear cells from patients with rheumatoid arthritis is associated with loss of radiation-induced apoptosis. Arthritis Rheum, 2005. 52(4): p. 1047-57.
61. Cameron, B. J., et al., Identification of a Titin-derived HLA-A1-presented peptide as a cross-reactive target for engineered MAGE A3-directed T cells. Sci Transl Med, 2013. 5(197): p. 197ra103.
62. Linette, G. P., et al., Cardiovascular toxicity and titin cross-reactivity of affinity-enhanced T cells in myeloma and melanoma. Blood, 2013. 122(6): p. 863-71.
63. Wittman, V. P., et al., Antibody targeting to a class I MHC-peptide epitope promotes tumor cell death. J Immunol, 2006. 177(6): p. 4187-95.
64. Sergeeva, A., et al., An anti-PR1/HLA-A2 T-cell receptor-like antibody mediates complement-dependent cytotoxicity against acute myeloid leukemia progenitor cells. Blood, 2011. 117(16): p. 4262-72.
65. Dao, T., et al., Targeting the intracellular WT1 oncogene product with a therapeutic human antibody. Sci Transl Med, 2013. 5(176): p. 176ra33.
66. Overdijk, M. B., et al., Crosstalk between human IgG isotypes and murine effector cells. J Immunol, 2012. 189(7): p. 3430-8.
67. Mason, D. Y., J. L. Cordell, and K. A. F. Pulford, Production of monoclonal antibodies for immunocytochemical use., in Techniques in Immunocytochemistry, G. R. Bullock and P. Petrusz, Editors. 1983, Academic Press: London. p. 175-216.
68. Tipton, T. R., et al., Antigenic modulation limits the effector cell mechanisms employed by type I anti-CD20 monoclonal antibodies. Blood, 2015.
69. Kabat et al., J. Immunol., 1991; 147(5): 1709-1719.
70. Al-Lazikani B., Lesk A. M., Chothia C. (1997). Standard conformations for the canonical structures of immunoglobulins. J Mol Biol. 273. 927-948.
71. Samaranayake et al., Ann. Med., 2009; 41(5): 322-31.
72. Fang et al., Mol. Ther., 2007; 15(6):1153-9.
73. Kershaw et al., Nat Rev Cancer., 2013; 13; 525-541
74. Kershaw et al., Clin Cancer Res., 2006; 15; 12: 6106-15.
75. Brentjens et al., Nat Med., 2003; 9; 3; 279-86.
76. Maude, S. L., et al., CD19-targeted chimeric antigen receptor T-cell therapy for acute lymphoblastic leukemia. Blood. 2015 125(26): p. 4017-23.
77. Cornelissen, B., et al., Imaging DNA damage in vivo using gammaH2AX-targeted immunoconjugates. Cancer Res. 2011 71(13): p. 4539-49.
78. Kawata et al. Establishment and characterization of the tumors of chronic lymphocytic leukemia cell line in nude and SCID mice. Leuk Res. 1993 October; 17(10):883-94.
79. Ohno et al., A newly established human lymphoma cell line, FL-18, carrying a 14;18 translocation. Jpn J Cancer Res. 1985 July; 76(7):563-6.
80. Mehra et al. Molecular cytogenetic characterization of non-Hodgkin lymphoma cell lines. Genes Chromosomes Cancer. 2002 March; 33(3):225-34.

1. An antibody which binds to human p53 tumour suppressor protein residues 65-73 (human p53₆₅₋₇₃), as shown in SEQ ID NO: 1, when presented by the MHC class I protein Human Leukocyte Antigen-A*0201 (HLA-A*0201).

2. An antibody according to claim 1, which binds to human p53₆₅₋₇₃ when presented by HLA-A*0201 with a dissociation constant (K_{D}) of 200 μM or less, preferably 150 μM or less, preferably 0.005-110 μM, preferably 0.01-110 μM, preferably 0.8-30 μM, more preferably 0.90-5.5 μM; wherein said dissociation constant has been determined by quartz crystal microbalance assay.

3. An antibody according to claim 1, which exhibits greater cell surface binding to T2 lymphoblast cells presenting the sequence of human p53₆₅₋₇₃ by HLA-A*0201, than a non-specific peptide, preferably consisting of the amino acid sequence of SEQ ID NO: 22.

4. An antibody according to claim 1, which binds to the cell surface of an HLA-A2+/p53+ cancer cell line; preferably any cell line selected from the group consisting of NCI-H2087 (lung), NCI-H1395 (lung), Hs-695T (melanoma), 143B (osteosarcoma), SW480 (colon), AU565 (breast), MDA-MB-231 (breast), MO-1043 (chronic lymphocytic leukaemia), FL-18 (follicular lymphoma), Granta-519 (mast cell leukaemia), OCI-Ly1 (diffuse large B-cell lymphoma) and OCI-Ly8 (diffuse large B-cell lymphoma).

5. An antibody according to claim 1, which, when applied in vitro to the surface of human B cell lymphoma cells at 10 μg/ml and subsequently incubated at 37° C., is internalised; preferably wherein the cells are of the cell line OCI-Ly8.

6. An antibody according to claim 1, which is able to elicit antibody-dependent cellular phagocytosis, antibody-dependent cellular cytotoxicity and complement-dependent cytotoxicity immune effector mechanisms.

7. An antibody according to claim 1, comprising a CDR-L1, L2 and L3 having the amino acid sequence of SEQ ID NO: 2, 3 and 4 respectively, or a CDR-L1, L2 and L3 having variant amino acid sequence of SEQ ID NO: 2, 3 and 4 respectively with no more than 4 substitutions, insertions and/or deletions relative to said sequences; further comprising
(1) a CDR-H1, H2 and H3 having the amino acid sequence of SEQ ID NO: 7, 8 and 9 respectively, or a CDR-H1, H2 and H3 having variant amino acid sequences of SEQ ID NO: 7, 8 and 9 respectively with no more than 4 substitutions, insertions and/or deletions relative to said sequences; or
(2) a CDR-H1, H2 and H3 having the amino acid sequence of SEQ ID NO: 7, 11 and 9 respectively, or a CDR-H1, H2 and H3 having variant amino acid sequences of SEQ ID NO: 7, 11 and 9 respectively with no more than 4 substitutions, insertions and/or deletions relative to said sequences; or
(3) a CDR-H1, H2 and H3 having the amino acid sequence of SEQ ID NO: 10, 11 and 9 respectively, or a CDR-H1, H2 and H3 having variant amino acid sequences of SEQ ID NO: 10, 11 and 9 respectively with no more than 4 substitutions, insertions and/or deletions relative to said sequences;
preferably;
(i) wherein the antibody of (1) comprises a light chain variable domain (V_{L}) having the amino acid sequence of SEQ ID NO: 12 or a variant amino acid sequence of SEQ ID NO: 12 having at least 70% identity thereto, and a heavy chain variable domain (V_{H}) having the sequence of SEQ ID NO: 17 or a variant amino acid sequence of SEQ ID NO: 17 having at least 70% identity thereto; or
(ii) wherein the antibody of (1) comprises a V_{L} having the sequence of SEQ ID NO: 13 or a variant amino acid sequence of SEQ ID NO: 13 having at least 70% identity thereto, and a V_{H} having the sequence of SEQ ID NO: 18 or a variant amino acid sequence of SEQ ID NO: 13 having at least 70% identity thereto; or
(iii) wherein the antibody of (1) comprises a V_{L} having the sequence of SEQ ID NO: 13 or a variant amino acid sequence of SEQ ID NO: 13 having at least 70% identity thereto, and a V_{H} having the sequence of SEQ ID NO: 19 or a variant amino acid sequence of SEQ ID NO: 19 having at least 70% identity thereto; or
(iv) wherein the antibody of (2) comprises a V_{L} having the sequence of SEQ ID NO: 13 or a variant amino acid sequence of SEQ ID NO: 13 having at least 70% identity thereto, and a V_{H} having the sequence of SEQ ID NO: 20 or a variant amino acid sequence of SEQ ID NO: 20 having at least 70% identity thereto; or
(v) wherein the antibody of (3) comprises a V_{L} having the sequence of SEQ ID NO: 13 or a variant amino acid sequence of SEQ ID NO: 13 having at least 70% identity thereto, and a V_{H} having the sequence of SEQ ID NO: 21 or a variant amino acid sequence of SEQ ID NO: 21 having at least 70% identity thereto.

8. A pharmaceutical composition comprising an antibody according to claim 1, or the means for its expression, and a pharmaceutically acceptable diluent, excipient and/or adjuvant; optionally together with at least one additional therapeutic agent.

9. An antibody according to claim 1, or the means for its expression, or a pharmaceutical composition according to claim 8, for use in therapy; preferably of a subject having the haplotype HLA-A*0201.

10. An antibody, means for expression, or composition for use according to claim 9, wherein said therapy is a combination therapy; preferably comprising administration of said antibody, means for expression or composition; and administration of at least one chemotherapeutic agent, at least one additional antibody, radiotherapy and/or at least one cytokine

11. An antibody, means for expression, or composition for use according to claim 9, in the therapy of a tumour/cancer; preferably selected from the group consisting of lung cancer, melanoma, osteosarcoma, colon cancer, breast cancer, chronic lymphocytic leukaemia, follicular lymphoma, mast cell leukaemia, diffuse large B-cell lymphoma, prostate cancer, pancreatic cancer, ovarian cancer and mantle cell lymphoma; more preferably selected from the group consisting of lung cancer, melanoma, osteosarcoma, colon cancer, breast cancer, chronic lymphocytic leukaemia, follicular lymphoma, mast cell leukaemia, diffuse large B-cell lymphoma, pancreatic cancer and mantle cell lymphoma.

12. An antibody or composition for use according to claim 11, as a delivery means for a drug, radioisotope, nanoparticle or further antibody to the cells of the tumour/cancer; wherein the antibody is conjugated to the drug, radioisotope, nanoparticle or further antibody respectively.

13. An antibody or composition for use according to claim 12, wherein the antibody is conjugated to a radioisotope and the subject is to undergo in vivo positron emission tomography (PET) imaging; or wherein the antibody is conjugated to an iron nanoparticle and the subject is to undergo in vivo magnetic resonance imaging (MRI); wherein said imaging is for the assessment of tumour cell and/or off-target binding of said antibody.

14. An antibody or composition for use according to claim 11, as a delivery means for an immune effector cell to the cells of the tumour/cancer, wherein the immune effector cell expresses a chimeric receptor comprising the antibody as a single chain variable fragment; preferably wherein the immune effector cell is a T cell.

15. A T cell expressing a chimeric receptor comprising, in the extracellular domain thereof, an antibody according to claim 1 as a single chain variable fragment.

16. A hybridoma comprising and/or secreting an antibody according to claim 1.

17. A cell or cell line expressing an antibody according to claim 1 in recombinant form.

18. A recombinant expression vector, capable of expressing an antibody according to claim 1.

19. Use of an antibody according to claim 1, in an in vitro method for determining the level of cellular antigen presentation of human p53₆₅₋₇₃ by HLA-A*0201.

20. An in vitro method for determining the suitability of a subject having a tumour/cancer to undergo immunotherapy; comprising contacting one or more cells obtained from the subject with an antibody according to claim 1, and determining the presence, absence or level of binding of said antibody to the surface of said one or more cells; wherein
(1) said one or more cells comprise tumour cells of the tumour/cancer; and wherein cell surface binding is a positive indication of the suitability of the subject to undergo said immunotherapy; or
(2) said one or more cells comprise non-malignant cells, and wherein cell surface binding is a negative indication of the suitability of the subject to undergo said immunotherapy;
wherein said immunotherapy is to be specific for human p53₆₅₋₇₃ presented by HLA-A*0201.


SEQUENCE LISTING (140 sequences)

SEQ ID NO: 1 - protein, 9 aa, Homo sapiens
RMPEAAPPV

SEQ ID NO: 2 - protein, 10 aa, Mus musculus
SASSSVSYIY

SEQ ID NO: 3 - protein, 7 aa, Mus musculus
STSNLAS

SEQ ID NO: 4 - protein, 9 aa, Mus musculus
QQRSNYPFT

SEQ ID NO: 5 - protein, 10 aa, Artificial Sequence
  Humanised and deimmunised VL_4; LCDR1
RASSSVSYIY

SEQ ID NO: 6 - protein, 7 aa, Artificial Sequence
  Humanised and deimmunsed VL_4; LCDR2
STSNRAT

SEQ ID NO: 7 - protein, 5 aa, Mus musculus
NYWIH

SEQ ID NO: 8 - protein, 17 aa, Mus musculus
NIYPSDSYSNYNQKFKD

SEQ ID NO: 9 - protein, 12 aa, Mus musculus
SEGYYDYEAFDY

SEQ ID NO: 10 - protein, 5 aa, Artificial Sequence
  Humanised and deimmunised VL_4; HCDR1
NYWMH

SEQ ID NO: 11 - protein, 17 aa, Artificial Sequence
  Humanised and deimmunised VH_2; HCDR2
NIYPSDSYTNYAQKFQG

SEQ ID NO: 12 - protein, 107 aa, Mus musculus
QIVLTQSPAIMSASPGEKVTVTCSASSSVSYIYWFQQKPGTSPKLWIYSTSNLASGVPARFSGSGSGTSYSLTISRMEAEDAATYYCQQRSNYPFTFGSGTKLEIKR

SEQ ID NO: 13 - protein, 107 aa, Artificial Sequence
  Humanised and deimmunised VL_1
QIVLTQSPATLSLSPGERATLSCSASSSVSYIYWFQQKPGQAPRLWIYSTSNLASGVPARFSGSGSGTDYTLTISSLEPEDAAVYYCQQRSNYPFTFGSGTKLEIKR

SEQ ID NO: 14 - protein, 107 aa, Artificial Sequence
  Humanised and deimmunised VL_2
QIVLTQSPATLSLSPGERATLSCSASSSVSYIYWYQQKPGQAPRLLIYSTSNLASGIPARFSGSGSGTDYTLTISSLEPEDFAVYYCQQRSNYPFTFGQGTKLEIKR

SEQ ID NO: 15 - protein, 107 aa, Artificial Sequence
  Humanised and deimmunised VL_3
QIVLTQSPATLSLSPGERATLSCSASSSVSYIYWYQQKPGQAPRLLIYSTSNRATGIPARFSGSGSGTDYTLTISSLEPEDFAVYYCQQRSNYPFTFGQGTKLEIKR

SEQ ID NO: 16 - protein, 107 aa, Artificial Sequence
  Humanised and deimmunised VL_4
QIVLTQSPATLSLSPGERATLSCRASSSVSYIYWYQQKPGQAPRLLIYSTSNRATGIPARFSGSGSGTDYTLTISSLEPEDFAVYYCQQRSNYPFTFGQGTKLEIKR

SEQ ID NO: 17 - protein, 121 aa, Mus musculus
EVQLQQSGAELVRPGASVKLSCKASGYTFTNYWIHWVKQRPGQGLEWIGNIYPSDSYSNYNQKFKDKATLTVDRSSSTAYMQLSSPTSEDSAVYYCARSEGYYDYEAFDYWGQGTTLTVSS

SEQ ID NO: 18 - protein, 121 aa, Artificial Sequence
  Humanised and deimmunised VH_1
EVQLVQSGAEVKKPGASVKVSCKASGYTFTNYWIHWVRQAPGQGLEWMGNIYPSDSYSNYNQKFKDRATLTVDRSTSTAYMELSSLRSEDTAVYYCARSEGYYDYEAFDYWGQGTLVTVSS

SEQ ID NO: 19 - protein, 121 aa, Artificial Sequence
  Humanised and deimmunised VH_2
QVQLVQSGAEVKKPGASVKVSCKASGYTFTNYWIHWVRQAPGQGLEWMGNIYPSDSYSNYAQKFQGRVTLTVDTSTSTAYMELSSLRSEDTAVYYCARSEGYYDYEAFDYWGQGTLVTVSS

SEQ ID NO: 20 - protein, 121 aa, Artificial Sequence
  Humanised and deimmunised VH_3
QVQLVQSGAEVKKPGASVKVSCKASGYTFTNYWIHWVRQAPGQGLEWMGNIYPSDSYSNYAQKFQGRVTITADTSTSTAYMELSSLRSEDTAVYYCARSEGYYDYEAFDYWGQGTLVTVSS

SEQ ID NO: 21 - protein, 121 aa, Artificial Sequence
  Humanised and deimmunised VH_4
QVQLVQSGAEVKKPGASVKVSCKASGYTFTNYWMHWVRQAPGQGLEWMGNIYPSDSYTNYAQKFQGRVTITADTSTSTAYMELSSLRSEDTAVYYCARSEGYYDYEAFDYWGQGTLVTVSS

SEQ ID NO: 22 - protein, 9 aa, Influenza A virus
GILGFVFTL

SEQ ID NO: 23 - protein, 9 aa, Homo sapiens
RAPAAAPFV

SEQ ID NO: 24 - protein, 9 aa, Homo sapiens
RAPCGAPSV

SEQ ID NO: 25 - protein, 9 aa, Homo sapiens
RAPSSAPTV

SEQ ID NO: 26 - protein, 9 aa, Homo sapiens
RDPKKAPSV

SEQ ID NO: 27 - protein, 9 aa, Homo sapiens
RDPPPAPPV

SEQ ID NO: 28 - protein, 9 aa, Homo sapiens
RDPSQAPPV

SEQ ID NO: 29 - protein, 9 aa, Homo sapiens
RDPVLAPAV

SEQ ID NO: 30 - protein, 9 aa, Homo sapiens
REPVRAPAV

SEQ ID NO: 31 - protein, 9 aa, Homo sapiens
RGPIAAPQV

SEQ ID NO: 32 - protein, 9 aa, Homo sapiens
RLPPTAPGV

SEQ ID NO: 33 - protein, 9 aa, Homo sapiens
RLPYTAPPV

SEQ ID NO: 34 - protein, 9 aa, Homo sapiens
RPPPGAPDV

SEQ ID NO: 35 - protein, 9 aa, Homo sapiens
RQPAMAPGV

SEQ ID NO: 36 - protein, 9 aa, Homo sapiens
RSPPAAPAV

SEQ ID NO: 37 - protein, 9 aa, Homo sapiens
RTPLSAPCV

SEQ ID NO: 38 - protein, 9 aa, Homo sapiens
RVPETAPEV

SEQ ID NO: 39 - protein, 9 aa, Homo sapiens
RVPKIAPDV

SEQ ID NO: 40 - protein, 9 aa, Homo sapiens
RVPMIAPRV

SEQ ID NO: 41 - protein, 9 aa, Homo sapiens
RYPIRAPRV

SEQ ID NO: 42 - DNA, 20 bp, Artificial Sequence
  Primer
gatgtgaagcttcaggagtc

SEQ ID NO: 43 - DNA, 20 bp, Artificial Sequence
  Primer
caggtgcagctgaaggagtc

SEQ ID NO: 44 - DNA, 20 bp, Artificial Sequence
  Primer
caggtgcagctgaagcagtc

SEQ ID NO: 45 - DNA, 20 bp, Artificial Sequence
  Primer
caggttactctgaaagagtc

SEQ ID NO: 46 - DNA, 21 bp, Artificial Sequence
  Primer
gaggtccagctgcaacaatct

SEQ ID NO: 47 - DNA, 19 bp, Artificial Sequence
  Primer
gaggtccagctgcagcagc

SEQ ID NO: 48 - DNA, 21 bp, Artificial Sequence
  Primer
caggtccaactgcagcagcct

SEQ ID NO: 49 - DNA, 20 bp, Artificial Sequence
  Primer
gaggtgaagctggtggagtc

SEQ ID NO: 50 - DNA, 20 bp, Artificial Sequence
  Primer
gaggtgaagctggtggaatc

SEQ ID NO: 51 - DNA, 20 bp, Artificial Sequence
  Primer
gatgtgaacttggaagtgtc

SEQ ID NO: 52 - DNA, 20 bp, Artificial Sequence
  Primer
gaggtccagctgcaacagtc

SEQ ID NO: 53 - DNA, 20 bp, Artificial Sequence
  Primer
gaggtgcagctggaggagtc

SEQ ID NO: 54 - DNA, 35 bp, Artificial Sequence
  Primer
gccagtggatagtcagatgggggtgtcgttttggc

SEQ ID NO: 55 - DNA, 21 bp, Artificial Sequence
  Primer
gatgttttgatgacccaaact

SEQ ID NO: 56 - DNA, 21 bp, Artificial Sequence
  Primer
gatattgtgatgacgcaggct

SEQ ID NO: 57 - DNA, 18 bp, Artificial Sequence
  Primer
gatattgtgataacccag

SEQ ID NO: 58 - DNA, 21 bp, Artificial Sequence
  Primer
gacattgtgctgacccaatct

SEQ ID NO: 59 - DNA, 21 bp, Artificial Sequence
  Primer
gacattgtgatgacccagtct

SEQ ID NO: 60 - DNA, 21 bp, Artificial Sequence
  Primer
gatattgtgctaactcagtct

SEQ ID NO: 61 - DNA, 21 bp, Artificial Sequence
  Primer
gatatccagatgacacagact

SEQ ID NO: 62 - DNA, 21 bp, Artificial Sequence
  Primer
gacatccagctgactcagtct

SEQ ID NO: 63 - DNA, 21 bp, Artificial Sequence
  Primer
caaattgttctcacccagtct

SEQ ID NO: 64 - DNA, 21 bp, Artificial Sequence
  Primer
gacattctgatgacccagtct

SEQ ID NO: 65 - DNA, 21 bp, Artificial Sequence
  Primer
ggatacagttggtgcagcatc

SEQ ID NO: 66 - protein, 9 aa, Artificial Sequence
  Synthetic sequence
LLGRNSFEV

SEQ ID NO: 67 - protein, 9 aa, Artificial Sequence
  Synthetic peptide.  Xaa can be any amino acid.
  Xaa (2)..(8): Xaa is an amino acid that can be substituted into the RAPAAAPFV p53(65-73) peptide without reducing T1-116C antibody binding by >50%.
RXPXXAPXV

SEQ ID NO: 68 - protein, 9 aa, Influenza A virus
GILGFTFVL

SEQ ID NO: 69 - protein, 11 aa, Homo sapiens
GLAPPQHLIRV

SEQ ID NO: 70 - protein, 11 aa, Mus musculus
GLAPPQHLIRV

SEQ ID NO: 71 - protein, 9 aa, Mus musculus
RVSGAPAAQ

SEQ ID NO: 72 - protein, 9 aa, Homo sapiens
EVDPIGHLY

SEQ ID NO: 73 - protein, 9 aa, Homo sapiens
ESDPIVAQY

SEQ ID NO: 74 - protein, 9 aa, Artificial Sequence
  Consensus binding sequence
RAPAAAPFV

SEQ ID NO: 75 - protein, 9 aa, Artificial Sequence
  Consensus binding sequence
  Xaa (2)..(2): Xaa can be Met, Ala, Cys, Gly, Ile, Leu, Gln, Ser, Thr, Val, or Trp
RXPEAAPPV

SEQ ID NO: 76 - protein, 9 aa, Artificial Sequence
  Consensus binding sequence
  Xaa (3)..(3): Xaa can be Pro, Ala, Cys, Asp, His, Leu, Met, Gln, Ser, Val, Trp, or Tyr
RMXEAAPPV

SEQ ID NO: 77 - protein, 9 aa, Artificial Sequence
  Consensus binding sequence
  Xaa (4)..(4): Xaa can be Glu, Ala, Cys, Asp, Gly, Lys, Met, Asn, Pro, Gln, Ser, or Val
RMPXAAPPV

SEQ ID NO: 78 - protein, 9 aa, Artificial Sequence
  Consensus binding sequence
  Xaa (5)..(5): Xaa can be any standard amino acid
RMPEXAPPV

SEQ ID NO: 79 - protein, 9 aa, Artificial Sequence
  Consensus binding sequence
  Xaa (6)..(6): Xaa can be Ala, Cys, Asp, Glu, Phe, Gly, His, Ile, Leu, Met, Asn, Pro, Gln, Ser, Thr, Val, Trp, Tyr
RMPEAXPPV

SEQ ID NO: 80 - protein, 9 aa, Artificial Sequence
  Consensus binding sequence
  Xaa (7)..(7): Xaa can be Pro, Ala, Cys, Asp, Glu, Phe, His, Ile, Lys, Leu, Met, Gln, Ser, Thr, Val, Trp, Tyr
RMPEAAXPV

SEQ ID NO: 81 - protein, 9 aa, Artificial Sequence
  Consensus binding sequence
  Xaa (8)..(8): Xaa can be Pro, Ala, Cys, Glu, Phe, Gly, His, Ile, Leu, Met, Asn, Gln, Arg, Ser, Val, Trp, or Tyr
RMPEAAPXV

SEQ ID NO: 82 - protein, 9 aa, Artificial Sequence
  Consensus binding sequence
  Xaa (9)..(9): Xaa can be Val, Ala, Cys, Ile or Leu
RMPEAAPPX

SEQ ID NO: 83 - protein, 9 aa, Homo sapiens
RLGPTLMCL

SEQ ID NO: 84 - protein, 9 aa, Homo sapiens
RIWSWLLGA

SEQ ID NO: 85 - protein, 9 aa, Homo sapiens
RLMKQDFSV

SEQ ID NO: 86 - protein, 9 aa, Homo sapiens
RMFPNAPYL

SEQ ID NO: 87 - protein, 9 aa, Homo sapiens
RLLEFYLAM

SEQ ID NO: 88 - protein, 13 aa, Artificial Sequence
  synthetic BirA biotinylation sequence
LNDIFEAQKIEWH

SEQ ID NO: 89 - protein, 9 aa, Artificial Sequence
  Synthetic sequence
AMPEAAPPV

SEQ ID NO: 90 - protein, 9 aa, Artificial Sequence
  Synthetic sequence
RAPEAAPPV

SEQ ID NO: 91 - protein, 9 aa, Artificial Sequence
  Synthetic sequence
RMAEAAPPV

SEQ ID NO: 92 - protein, 9 aa, Artificial Sequence
  Synthetic sequence
RMPAAAPPV

SEQ ID NO: 93 - protein, 9 aa, Artificial Sequence
  Synthetic sequence
RMPEAAAPV

SEQ ID NO: 94 - protein, 9 aa, Artificial Sequence
  Synthetic sequence
RMPEAAPAV

SEQ ID NO: 95 - protein, 9 aa, Artificial Sequence
  Synthetic sequence
RMPEAAPPA

SEQ ID NO: 96 - protein, 9 aa, Artificial Sequence
  Synthetic sequence
GMPEAAPPV

SEQ ID NO: 97 - protein, 9 aa, Artificial Sequence
  Synthetic sequence
RGPEAAPPV

SEQ ID NO: 98 - protein, 9 aa, Artificial Sequence
  Synthetic sequence
RMGEAAPPV

SEQ ID NO: 99 - protein, 9 aa, Artificial Sequence
  Synthetic sequence
RMPGAAPPV

SEQ ID NO: 100 - protein, 9 aa, Artificial Sequence
  Synthetic sequence
RMPEGAPPV

SEQ ID NO: 101 - protein, 9 aa, Artificial Sequence
  Synthetic sequence
RMPEAGPPV

SEQ ID NO: 102 - protein, 9 aa, Artificial Sequence
  Synthetic sequence
RMPEAAGPV

SEQ ID NO: 103 - protein, 9 aa, Artificial Sequence
  Synthetic sequence
RMPEAAPGV

SEQ ID NO: 104 - protein, 9 aa, Artificial Sequence
  Synthetic sequence
RMPEAAPPG

SEQ ID NO: 105 - protein, 9 aa, Artificial Sequence
  Synthetic sequence
  Xaa (1)..(1): Xaa can be any standard amino acid
XMPEAAPPV

SEQ ID NO: 106 - protein, 9 aa, Homo sapiens
RAPAAAPFV

SEQ ID NO: 107 - protein, 9 aa, Homo sapiens
RAPCGAPSV

SEQ ID NO: 108 - protein, 9 aa, Homo sapiens
RAPSSAPTV

SEQ ID NO: 109 - protein, 9 aa, Homo sapiens
RDPKKAPSV

SEQ ID NO: 110 - protein, 9 aa, Homo sapiens
RDPPPAPPV

SEQ ID NO: 111 - protein, 9 aa, Homo sapiens
RDPVLAPAV

SEQ ID NO: 112 - protein, 9 aa, Homo sapiens
REPVRAPAV

SEQ ID NO: 113 - protein, 9 aa, Homo sapiens
RPPPGAPDV

SEQ ID NO: 114 - protein, 9 aa, Homo sapiens
RQPAMAPGV

SEQ ID NO: 115 - protein, 9 aa, Homo sapiens
RSPPAAPAV

SEQ ID NO: 116 - protein, 9 aa, Homo sapiens
RTPLSAPCV

SEQ ID NO: 117 - protein, 9 aa, Homo sapiens
RVPETAPEV

SEQ ID NO: 118 - protein, 9 aa, Homo sapiens
RYPIRAPRV

SEQ ID NO: 119 - protein, 9 aa, Homo sapiens
RDPSQAPPV

SEQ ID NO: 120 - protein, 9 aa, Homo sapiens
RGPIAAPQV

SEQ ID NO: 121 - protein, 9 aa, Homo sapiens
RLPPTAPGV

SEQ ID NO: 122 - protein, 9 aa, Homo sapiens
RLPYTAPPV

SEQ ID NO: 123 - protein, 9 aa, Homo sapiens
RVPKIAPDV

SEQ ID NO: 124 - protein, 9 aa, Homo sapiens
RVPMIAPRV

SEQ ID NO: 125 - protein, 9 aa, Mus musculus
RDPSQAPPV

SEQ ID NO: 126 - protein, 9 aa, Mus musculus
RGPIAAPQV

SEQ ID NO: 127 - protein, 9 aa, Mus musculus
RLPPTAPGV

SEQ ID NO: 128 - protein, 9 aa, Mus musculus
RLPYTAPPV

SEQ ID NO: 129 - protein, 9 aa, Mus musculus
RVPKIAPDV

SEQ ID NO: 130 - protein, 9 aa, Mus musculus
RVPMIAPRV

SEQ ID NO: 131 - protein, 9 aa, Mus musculus
RAPNAAPEV

SEQ ID NO: 132 - protein, 9 aa, Mus musculus
RAPSPAPAV

SEQ ID NO: 133 - protein, 9 aa, Mus musculus
RDPTAAPYV

SEQ ID NO: 134 - protein, 9 aa, Mus musculus
RGPVRAPGV

SEQ ID NO: 135 - protein, 9 aa, Mus musculus
RGPVRAPQV

SEQ ID NO: 136 - protein, 9 aa, Mus musculus
RPPLPAPAV

SEQ ID NO: 137 - protein, 9 aa, Mus musculus
RQPAVAPGV

SEQ ID NO: 138 - protein, 9 aa, Mus musculus
RSPRTAPPV

SEQ ID NO: 139 - protein, 9 aa, Mus musculus
RTPCGAPSV

SEQ ID NO: 140 - protein, 9 aa, Mus musculus
RTPNEAPSV